(19) United States
(12) Patent Application Publication
(10) Pub. No.: US 2021/0008106 A1
(43) Pub. Date: Jan. 14, 2021

(54) MITOCHONDRIAL TREATMENT OF ORGANS FOR TRANSPLANTATION

(71) Applicant: United Therapeutics Corporation, Silver Spring, MD (US)

(72) Inventors: Thomas Petersen, Durham, NC (US); Sarah Hogan, Raleigh, NC (US); Roger Ilagan, Durham, NC (US); Caryn Cloer, Advance, NC (US)

(73) Assignee: United Therapeutics Corporation, Silver Spring, MD (US)

(21) Appl. No.: 16/903,975

(22) Filed: Jun. 17, 2020

Related U.S. Application Data
(60) Provisional application No. 62/863,034, filed on Jun. 18, 2019.

(51) Int. Cl. A61K 35/12; C12N 5/071; A61P 11/00

(52) U.S. Cl. CPC A61K 35/12; A61P 11/00; C12N 5/0602

(57) ABSTRACT

Methods and compositions relating to isolated mitochondria are disclosed. For example, cells, tissues, or organs can be treated with isolated mitochondria, such as porcine mitochondria, to improve mitochondrial function in the cell, tissue, or organ. The improvements to mitochondrial function include increased oxygen consumption and increased ATP synthesis. Such methods and compositions are useful for cell therapy; organ and tissue transplantation; organ and tissue engineering; and cold storage or shipment of harvested organs, tissues, and cells.

### CROSS-REFERENCE TO RELATED APPLICATIONS

This application claims priority to U.S. Provisional Application No. 62/863,034, filed Jun. 18, 2019.

### FIELD OF THE INVENTION

This disclosure relates to the use of mitochondria, such as isolated porcine mitochondria or isolated human mitochondria, for improving cell, tissue, and organ function and to the therapeutic use of mitochondria.

### BACKGROUND OF THE INVENTION

The mitochondrion is a double-membrane-bound organelle in eukaryotic cells that plays a key role in the maintenance and preservation of cellular homeostasis and function. For example, mitochondria supply cellular energy and play a key role in cell signaling, cellular differentiation, cellular apoptosis, cell cycle regulation, and cell growth. Typically, mitochondria supply more than 90% of a cell's ATP requirement.

The mitochondrion is composed of an outer mitochondrial membrane, an inner mitochondrial membrane, an intermembrane space between the outer and inner membranes, the cristae space formed by infoldings of the inner membrane, the matrix space within the inner membrane, a mitochondria-associated ER membrane (MAM), and an independent genome within the matrix that shows substantial similarity to bacterial genomes. The outer mitochondrial membrane contains integral membrane proteins called porins, which allow low molecular weight molecules to freely diffuse across the membrane, as well as enzymes involved in a diverse array of activities such as the elongation of fatty acids, oxidation of epinephrine, and the degradation of tryptophan. Disruption of the outer mitochondrial membrane results in the leaking of mitochondrial proteins into the cytosol, which triggers cell death by apoptosis. The inner mitochondrial membrane is a highly impermeable, protein rich membrane that performs the redox reactions of oxidative phosphorylation and contains ATP synthase, which generates ATP in the matrix.

Mitochondrial injury and loss of function are deleterious to a cell, tissue, or organ and have been implicated in both acquired and hereditary human diseases, including cardiac dysfunction, heart failure, and autism. Mitochondrial dysfunction occurs by a variety of mechanisms, including genetic alterations in nuclear or mitochondrial genomic DNA, ischemia, environmental insult, proinflammatory cytokines, reactive oxygen species (ROS) generated by activated immune cells, and conditions associated with oxidative stress. See, e.g., Rossignol, D. and R. Frye, Mol Psychiatry 2012, 17:389-401; Suematsu, N. et al., Circulation 2003, 107:1418-23; and Fernandez-Checa, J. et al., Am J Physiol. 1997, 273:G7-17, each of which is incorporated by reference herein in its entirety. For example, it has been shown that ischemia decreases mitochondrial complex activity, oxygen consumption, oxidoreductase activity, fatty acid and glucose metabolism, and adenosine triphosphate (ATP) synthesis and increases calcium accumulation. See, e.g., Faulk, E. et al., Circulation 1995, 92:405-12; Black, K. et al., Physiol Genomics 2012, 44:1027-41; and Masuzawa, A. et al., Am J Physiol Heart Circ Physiol. 2013, 304:H966-82, each of which is incorporated by reference herein in its entirety. Diseases caused by mutations in mitochondrial DNA include Leber's hereditary optic neuropathy, MELAS syndrome, and Kearns-Sayre syndrome.

Because of the crucial role mitochondria play in cell metabolism, improving mitochondrial function could promote viability and function of cells, tissues, and organs under conditions of stress such as during cold exposure and ischemia. It has previously been shown by McCully et al. (Mitochondrion 2017, 34:127-34, which is incorporated by reference herein in its entirety) that transplantation of autologous mitochondria (i.e., mitochondria isolated from a patient's own body) decreased myocardial injury resulting from transient ischemia. Currently, however, there are no known and approved treatments or therapies that involve the treatment of cells, tissue, or organs with exogenous mitochondria, such as porcine mitochondria or exogenous human mitochondria (i.e., mitochondria isolated from a first human subject used to treat the cells, tissue, or organs of a second human subject).

Thus, there is a continuing need in the fields of cell therapy, transplantation, and organ/tissue engineering for exogenous mitochondria that can be obtained from a readily available source and are capable of improving the function and viability of cells, tissues, or organs. Such exogenous mitochondria would find utility in improving the efficacy and efficiency of organ transplantation and engineering, for example improving lung function during ex vivo lung perfusion (EVLP). Such exogenous mitochondria would also find utility in minimizing cell damage and inflammation associated with hypoxia and cold ischemia, for example cell damage and inflammation incurred during cold storage or shipment of harvested organs, tissues, or cells.

### SUMMARY OF THE INVENTION

This present disclosure relates to the use of mitochondria for improving cell, tissue, or organ function and to the therapeutic use of mitochondria. Mitochondria can be isolated from any suitable source including, but not limited to, cells or tissue obtained from a mammalian donor. Non-limiting examples of mammalian donors are humans, non-human primates, pigs, sheep, canines, rabbits, mice, and rats. The present disclosure frequently refers to the use of porcine mitochondria, but it should be understood that any suitable mitochondria can be used. Thus, when the disclosure, other than the claims, refers to “porcine mitochondria,” it is to be understood that the mitochondria can also be mitochondria from human or other non-human sources.

In some embodiments, the mitochondria are exogenous mitochondria. In some embodiments, the exogenous mitochondria are xenogeneic with respect to the target cell, tissue, or organ. In some embodiments, the exogenous mitochondria are allogeneic with respect to the target cell, tissue, or organ. In some embodiments, the mitochondria are endogenous mitochondria. In some embodiments, the mitochondria are autologous mitochondria. In preferred embodiments, porcine mitochondria are used to treat a human cell, tissue, or organ. In some embodiments, the porcine mitochondria are isolated from a porcine subject genetically engineered for use in organ transplantation in humans. In other preferred embodiments, mitochondria isolated from a first human subject are used to treat a human cell, tissue, or organ from a second human subject. In some embodiments, the human mitochondria are isolated from the donor of a cell, tissue, or organ intended for transplantation. In some embodiments, the human mitochondria are isolated from a recipient of a cell, tissue, or organ transplant. In some embodiments, the human mitochondria are isolated from an intended recipient of a cell, tissue, or organ transplant. In some embodiments, the human mitochondria are allogeneic to the intended recipient of a cell, tissue, or organ transplant. In some embodiments, the human mitochondria are autologous to the intended recipient of a cell, tissue, or organ transplant. In some embodiments, the cell, tissue, or organ intended for transplantation is treated with mitochondria allogeneic to the cell, tissue, or organ intended for transplantation. In some embodiments, the cell, tissue, or organ intended for transplantation is treated with mitochondria autologous to the cell, tissue, or organ intended for transplantation.

The present disclosure provides methods of organ transplantation comprising delivering isolated mitochondria to an organ intended for transplantation. In another embodiment, the disclosure provides methods of improving the performance of an implanted tissue or transplanted organ in a subject comprising delivering isolated mitochondria to a tissue or organ before, during, or after implantation or transplantation of the tissue or organ, where the tissue or organ is a donor tissue, donor organ, engineered tissue, or engineered organ. In another embodiment, the disclosure provides methods of improving the function of a lung during ex vivo lung perfusion (EVLP) comprising: (i) delivering isolated mitochondria to a lung, and (ii) performing EVLP on the lung in a chamber or vessel by perfusing the lung with a perfusate solution from a reservoir. In another embodiment, the disclosure provides methods for minimizing damage to an organ ex vivo due to cold ischemia during transportation, shipment, or storage comprising: delivering isolated mitochondria to the organ 0-24 hours before cold ischemia, during cold ischemia, or 0-24 hours after cold ischemia, wherein cells of the organ treated with the isolated mitochondria have at least 5% improvement in mitochondrial function in comparison to cells of a corresponding organ not treated with the isolated mitochondria, and wherein the improved mitochondrial function is increased oxygen consumption and/or increased ATP synthesis.

In another embodiment, the disclosure provides methods for improving the function of an engineered organ or tissue comprising: (i) preparing an organ or tissue scaffold comprising one or more extracellular matrix components, (ii) populating the organ or tissue scaffold in a bioreactor, chamber, or vessel with populating cells to produce an engineered organ or tissue, and (iii) delivering isolated mitochondria to the engineered organ or tissue. In another embodiment, the disclosure provides methods for improving the function of an engineered organ or tissue comprising: (i) preparing an organ or tissue scaffold comprising one or more extracellular matrix components, and (ii) populating the organ or tissue scaffold in a bioreactor, chamber, or vessel with the populating cells treated with isolated mitochondria to produce an engineered organ or tissue. In another embodiment, the disclosure provides methods for improving the function of an engineered organ or tissue comprising: (i) preparing an organ or tissue scaffold comprising one or more extracellular matrix components, (ii) infusing the organ or tissue scaffold with the isolated mitochondria, and (iii) populating the infused organ or tissue scaffold in a bioreactor, chamber, or vessel with populating cells to produce an engineered organ or tissue.

In another embodiment, the disclosure provides methods for improving the function of an engineered lung comprising: (i) repopulating a decellularized scaffold lung in a bioreactor, chamber, or vessel with repopulating cells to produce an engineered lung, and (ii) delivering isolated mitochondria to the engineered lung. In another embodiment, the disclosure provides methods for improving the function of an engineered lung comprising: (i) delivering isolated mitochondria to repopulating cells, and (ii) repopulating a decellularized scaffold lung in a bioreactor, chamber, or vessel with the repopulating cells treated with the isolated mitochondria to produce an engineered lung.

In another embodiment, the disclosure provides methods for improving the function of an engineered kidney comprising: (i) repopulating a decellularized scaffold kidney in a bioreactor, chamber, or vessel with repopulating cells to produce an engineered kidney, and (ii) delivering isolated mitochondria to the engineered kidney. In another embodiment, the disclosure provides methods for improving the function of an engineered kidney comprising: (i) delivering isolated mitochondria to repopulating cells, and (ii) repopulating a decellularized scaffold kidney in a bioreactor, chamber, or vessel with the repopulating cells treated with the isolated mitochondria to produce an engineered kidney.

In another embodiment, the disclosure provides methods for treating a lung disease or disorder in a subject in need thereof or for improving the function of a donor lung prior to or after transplantation, the method comprising administering to the subject or donor lung a pharmaceutical composition comprising a mesenchymal stem cell or endothelial progenitor cell that has been pre-treated with isolated mitochondria, or extracellular vesicles isolated from the mesenchymal stem cell or endothelial progenitor cell. In another embodiment, the disclosure provides methods for treating a lung disease or disorder in a subject in need thereof or for improving the function of a donor lung prior to or after transplantation, the method comprising administering to the subject or donor lung (A) a mesenchymal stem cell or endothelial progenitor cell, or extracellular vesicles isolated from the mesenchymal stem cell or endothelial progenitor cell, and (B) isolated mitochondria, wherein (A) and (B) are comprised in a single pharmaceutical composition or two separate pharmaceutical compositions. In another embodiment, the disclosure provides methods for treating a lung disease or disorder in a subject in need thereof comprising: (i) administering a therapeutically effective amount of a composition comprising isolated mitochondria to the subject, and (ii) administering a therapeutically effective amount of a medication for treating the lung disease or disorder, wherein the composition is administered to the subject before, concurrently with, or after the administration of the medication for treating the lung disease or disorder. In another embodiment, the disclosure provides methods for treating pulmonary hypertension in a subject in need thereof comprising: (i) administering a therapeutically effective amount of a composition comprising isolated mitochondria to the subject, and (ii) administering a therapeutically effective amount of treprostinil, wherein the composition is administered to the subject before, concurrently with, or after the administration of treprostinil.

In another embodiment, the disclosure provides methods for treating a lung disease or disorder of a subject in need thereof or for improving the function of a donor lung prior to or after transplantation, the method comprising: (i) administering a therapeutically effective amount of a composition comprising isolated mitochondria to the subject or donor lung, and (ii) administering a therapeutically effective amount of UNEX-42 to the subject or donor lung, wherein the composition is administered to the subject or donor lung before, concurrently with, or after the administration of UNEX-42. In another embodiment, the disclosure provides methods for treating a lung disease or disorder in a subject in need thereof or for improving the function of a donor lung prior to or after transplantation, the method comprising: (i) administering a therapeutically effective amount of a composition comprising isolated mitochondria to the subject or donor lung, and (ii) administering a therapeutically effective amount of an anti-oxidant to the subject or donor lung, wherein the composition is administered to the subject or donor lung before, concurrently with, or after the administration of the anti-oxidant. In another embodiment, the disclosure provides methods for treating an acute exacerbation of a lung disease or disorder in a subject comprising administering an effective amount of a composition comprising isolated mitochondria to the subject for rescue therapy. In another embodiment, the disclosure provides methods for treating acute kidney injury in a subject in need thereof comprising administering a therapeutically effective amount of a composition comprising isolated mitochondria to the subject. In another embodiment, the disclosure provides methods for treating a subject in cardiac arrest or undergoing resuscitation comprising administering an effective amount of a composition comprising isolated mitochondria to the subject to facilitate transport thereof to a medical facility or medical treatment.

In another embodiment, the disclosure provides methods of preserving a tissue or organ for transportation and transplantation comprising delivering isolated mitochondria to a tissue or organ intended for transportation and transplantation, wherein the tissue or organ is procured from a deceased donor. In another embodiment, the disclosure provides methods of preserving a limb or other body part lost due to traumatic amputation comprising delivering isolated mitochondria to the limb or other body part after the traumatic amputation of the limb or other body part.

In another embodiment, the disclosure provides methods of reducing inflammation in a subject in need thereof comprising: (i) delivering isolated mitochondria to isolated hematopoietic lineage cells from the subject, and (ii) administering the hematopoietic lineage cells treated with the isolated mitochondria to the subject.

In another embodiment, the disclosure provides methods of improving the cellular function of isolated cells comprising delivering isolated mitochondria to the isolated cells.

In another embodiment, the disclosure provides methods of improving cell therapy in a subject in need thereof comprising: (i) delivering isolated mitochondria to isolated cells in vitro, and (ii) administering the cells treated with the isolated mitochondria to the subject.

In another embodiment, the disclosure provides methods for improving the cold transportation, cold shipment, or cold storage of isolated cells comprising delivering isolated mitochondria to the isolated cells before, during, or after cold transportation, cold shipment, or cold storage, wherein the cells treated with the isolated mitochondria have at least 5% improvement in viability in comparison to corresponding cells not treated with the isolated mitochondria. In another embodiment, the disclosure provides methods for cryopreservation of isolated mitochondria comprising freezing isolated mitochondria in a freezing buffer comprising a cryprotectant. In another embodiment, the disclosure provides methods for long-term storage of isolated mitochondria comprising (i) isolating mitochondria from cells or tissue, (ii) suspending the isolated mitochondria in a cold storage buffer, (iii) freezing the isolated mitochondria at a temperature from about −70° C. to about −100° C., and (iv) maintaining the frozen isolated mitochondria at a temperature from about −70° C. to about −100° C. for 24 hours or longer. The storage period can be at least 24 hours, at least one week, at least four weeks, at least three months, at least six months, at least 9 months, or at least 1 year.

In another embodiment, the disclosure provides methods for detecting porcine mitochondria in a human cell, tissue, or organ sample comprising detecting in vitro or ex vivo the presence of a nucleic acid marker in the human cell, tissue, or organ sample, wherein the nucleic acid marker comprises a sequence of mitochondrial DNA or RNA, and wherein the nucleic acid marker is present in porcine mitochondria and absent in human mitochondria.

In another embodiment, the disclosure provides compositions comprising human cells, wherein the cytosol of the human cells comprises exogenous mitochondria, wherein the human cells of the composition have at least 5% improvement in mitochondrial function in comparison to corresponding human cells lacking exogenous mitochondria, and wherein the improved mitochondrial function is increased oxygen consumption and/or increased ATP synthesis.

Further objects and advantages of the present invention will be clear from the description that follows.

### BRIEF DESCRIPTION OF THE DRAWINGS

FIG. 1 (FIG. 1) shows that treatment of human pulmonary artery endothelial cells (HPAEC) with mitochondria isolated from pig hearts (i.e., porcine mitochondria) increases the oxygen consumption rate (OCR) after acute cold exposure. HPAEC were placed in 4° C. for 6 hours. HPAEC recovered in normoxia for 1 hour at 37° C. in the presence of either 20 μL of mitochondria suspension (respiration buffer containing 29 particles per cell; “+ MITO”) or 20 μL of respiration buffer only (“− MITO”) and equilibrated in a non-CO₂ incubator for 10 minutes. A “Mitochondrial Stress Test” was then performed using a Seahorse instrument with 10 μM oligomycin, 20 μM FCCP, and 5 μM rotenone/antimycin A (Rot/AA). Porcine mitochondria treatment increased OCR at baseline (43.6% increase), oligomycin-treated HPAEC (204.9% increase), FCCP-treated HPAEC (8.4% increase), and Rot/AA-treated HPAEC (34.1% increase) in comparison to the corresponding baseline, oligomycin-treated, FCCP-treated, or Rot/AA-treated “−MITO” HPAEC control. Statistical analysis performed was a two-tailed t-test (* p<0.05; ** p<0.01).

FIG. 2 (FIG. 2) shows that porcine mitochondria treatment of human pulmonary artery endothelial cells (HPAEC) increases OCR after chronic cold exposure. HPAEC were placed in 4° C. for 12 hours. HPAEC recovered in normoxia for 1 hour at 37° C. in the presence of 20 μL of mitochondria suspension (respiration buffer containing 172 particles per cell; “+ MITO”) or 20 μL of respiration buffer only (“− MITO”) and equilibrated in a non-CO₂ incubator for 50 minutes. HPAEC were rested in the Seahorse instrument at 37° C. under non-CO₂ conditions. A “Mitochondrial Stress Test” was then performed with the Seahorse instrument with 10 μM oligomycin, 20 μM FCCP, and 5 rotenone/antimycin A (Rot/AA). Porcine mitochondria treatment increased OCR at baseline (32.4% increase), oligomycin-treated HPAEC (51.9% increase), FCCP-treated HPAEC (9.5% increase), and Rot/AA-treated HPAEC (45.2% increase) in comparison to the corresponding baseline, oligomycin-treated, FCCP-treated, or Rot/AA-treated “-MITO” HPAEC control. Statistical analysis performed was a two-tailed t-test (** p<0.01).

FIG. 3 (FIG. 3) shows that HPAEC exposed to cold stress take up porcine mitochondria. Porcine mitochondria were administered to HPAEC undergoing cold stress. For the cold recovery group, HPAEC under cold stress take up the porcine mitochondria in a dose-dependent manner, and maximal expression of porcine MtND5 is achieved at 1,666 particles per cell. In the cold recovery condition, maximal expression of porcine MtND5 is achieved at 24 hours, where a 26,201% increase in porcine MtND5 was observed compared to the untreated cold-recovery control. In the cold exposure condition, maximal expression of porcine MtND5 is achieved at 72 hours where a 301,932% increase in MtND5 was observed compared to the untreated cold-exposure control. Statistical analysis performed was a one-way ANOVA (* P<0.05 24 hour compared to normoxia; # P<0.05 48 hour compared to normoxia; + P<0.05 72 hour compared to normoxia; $ P<0.05 24 hour compared to cold control; \hat{ } P<0.05 48 hour compared to cold control; & P<0.05 72 hour compared to cold control).

FIG. 4 (FIG. 4) shows that transcription of human mitochondrial DNA in HPAEC exposed to cold stress is largely unaffected by porcine mitochondria treatment. Untreated control HPAEC under cold recovery conditions demonstrated a 55% increase in human MtND5 expression compared to normothermic controls. This increase was moderated by porcine mitochondria treatment, where 1 particle/cell demonstrated a 3.8% reduction in expression compared to untreated normothermic HPAEC and a 33% reduction in expression compared to the untreated cold-recovery control. In the cold exposure group, maximal expression of human MtND5 was achieved at 72 hours, but this increase was not significantly impacted by porcine mitochondria treatment. Statistical analysis performed was a one-way ANOVA (* P<0.05 24 hour compared to normoxia; # P<0.05 48 hour compared to normoxia; + P<0.05 72 hour compared to normoxia; $ P<0.05 24 hour compared to cold control; \hat{ } P<0.05 48 hour compared to cold control; & P<0.05 72 hour compared to cold control).

FIG. 5 (FIG. 5) shows that porcine mitochondria treatment of HPAEC reduces NF-κB expression in cold recovery at 24 hours. In the cold recovery condition, untreated control HPAEC demonstrated an 83% increase in NF-κB gene expression at 24 hours compared to normothermic controls. Porcine mitochondria treatment trended to decrease NF-κB expression compared to untreated cold-recovery control HPAEC, with 1 particle/cell demonstrating a 22% decrease compared to untreated cold-recovery control HPAEC. In the cold exposure condition, a slight increase in NF-κB expression occurs at 24 hours in HPAEC treated with porcine mitochondria, but this increase is not statistically significant. Statistical analysis performed was a one-way ANOVA (* P<0.05 24 hour compared to normoxia; # P<0.05 48 hour compared to normoxia; + P<0.05 72 hour compared to normoxia; $ P<0.05 24 hour compared to cold control; \hat{ } P<0.05 48 hour compared to cold control; & P<0.05 72 hour compared to cold control).

FIG. 6 (FIG. 6) shows that porcine mitochondria treatment of HPAEC decreases toll-like receptor-9 (TLR-9) expression in cold recovery after 24 hours. HPAEC were treated, cultured under cold recovery or cold exposure conditions, and harvested at 24-hour, 48-hour, or 72-hour time points. In the cold recovery condition, untreated control HPAEC demonstrated a 101% increase in TLR-9 expression at 24 hours compared to normothermic controls. Porcine mitochondria treatment trended to decrease the TLR-9 expression compared to untreated cold-recovery control HPAEC, with 166 particles/cell demonstrating a 37% decrease compared to untreated cold-recovery control HPAEC. In cold exposure conditions, maximal expression of TLR-9 occurs in HPAEC treated with 1 particle/cell, where a 60% increase in TLR-9 expression was observed compared to the untreated cold-exposure control HPAEC. Statistical analysis performed was a one-way ANOVA (* P<0.05 24 hour compared to normoxia; # P<0.05 48 hour compared to normoxia; + P<0.05 72 hour compared to normoxia; $ P<0.05 24 hour compared to cold control; \hat{ } P<0.05 48 hour compared to cold control; & P<0.05 72 hour compared to cold control).

FIG. 7 (FIG. 7) shows that porcine mitochondria treatment of HPAEC impacts the expression of heme oxygenase-1 (HO-1) in cold exposure at 24 hours. Porcine mitochondria treatment increased HO-1 expression in the cold exposure condition. Porcine mitochondria treatment was maximally effective at 16 particles/cell, where a 24% increase in HO-1 expression was seen compared to untreated cold-exposure control HPAEC (242% increase compared to untreated normothermic control HPAEC).

Statistical analysis performed was a one-way ANOVA (* P<0.05 24 hour compared to normoxia; # P<0.05 48 hour compared to normoxia; + P<0.05 72 hour compared to normoxia; $ P<0.05 24 hour compared to cold control; \hat{ } P<0.05 48 hour compared to cold control; & P<0.05 72 hour compared to cold control).

FIG. 8 (FIG. 8) shows that porcine mitochondria treatment of HPAEC decreases macrophage-colony stimulating factor (M-CSF) secretion under hypoxic conditions. Porcine mitochondria treatment is maximally effective at 3 particles/cell, where M-CSF secretion was reduced by 65% compared to untreated hypoxia control HPAEC at 48 hours. Statistical analysis performed was a one-way ANOVA (* P<0.05 24 hour compared to normoxia; # P<0.05 48 hour compared to normoxia; + P<0.05 72 hour compared to normoxia; $ P<0.05 24 hour compared to hypoxia control; \hat{ } P<0.05 48 hour compared to hypoxia control; & P<0.05 72 hour compared to hypoxia control).

FIG. 9 (FIG. 9) shows that porcine mitochondria treatment of HPAEC decreases macrophage inflammatory protein-1β (MIP-1β) secretion under hypoxic conditions. Porcine mitochondria treatment was maximally effective in reducing MIP-1β secretion at 3 particles/cell, where MIP-1β secretion was reduced by 73% compared to untreated hypoxia control HPAEC at 48 hours. A decrease in potency is seen at 3,687 particles/cell. Statistical analysis performed was a one-way ANOVA (* P<0.05 24 hour compared to normoxia; # P<0.05 48 hour compared to normoxia; + P<0.05 72 hour compared to normoxia; $ P<0.05 24 hour compared to hypoxia control; \hat{ } P<0.05 48 hour compared to hypoxia control; & P<0.05 72 hour compared to hypoxia control).

FIG. 10 (FIG. 10) shows that porcine mitochondria treatment of HPAEC decreases platelet-derived growth factor-BB (PDGF-BB) secretion under hypoxic conditions. Porcine mitochondria treatment was maximally effective in reducing PDGF-BB secretion at 36 particles/cell, where PDGF-BB secretion was reduced by 69% compared to untreated hypoxia control HPAEC at 48 hours. A decrease in potency is seen at 3,687 particles/cell. Statistical analysis performed was a one-way ANOVA (* P<0.05 24 hour compared to normoxia; # P<0.05 48 hour compared to normoxia; +P<0.05 72 hour compared to normoxia; $ P<0.05 24 hour compared to hypoxia control; \hat{ } P<0.05 48 hour compared to hypoxia control; & P<0.05 72 hour compared to hypoxia control).

FIG. 11 (FIG. 11) shows that porcine mitochondria treatment of HPAEC decreases RANTES (CCL5) secretion under hypoxic conditions. Porcine mitochondria treatment was maximally effective in reducing RANTES secretion at 0.3 particles/cell, where RANTES secretion was reduced by 59% compared to untreated hypoxia control HPAEC at 48 hours. A decrease in potency is seen at 3,687 particles/cell. Statistical analysis performed was a one-way ANOVA (* P<0.05 24 hour compared to normoxia; # P<0.05 48 hour compared to normoxia; + P<0.05 72 hour compared to normoxia; $ P<0.05 24 hour compared to hypoxia control; \hat{ } P<0.05 48 hour compared to hypoxia control; & P<0.05 72 hour compared to hypoxia control).

FIG. 12 (FIG. 12) shows that porcine mitochondria treatment of HPAEC decreases intracellular adhesion molecule-1 (ICAM-1) secretion under hypoxic conditions. Porcine mitochondria treatment was maximally effective in reducing ICAM-1 secretion at 0.3 particles/cell, where ICAM-1 secretion was reduced by 82% compared to untreated hypoxia control HPAEC at 48 hours. A decrease in potency is seen at 3,687 particles/cell. Statistical analysis performed was a one-way ANOVA (* P<0.05 24 hour compared to normoxia; # P<0.05 48 hour compared to normoxia; + P<0.05 72 hour compared to normoxia; $ P<0.05 24 hour compared to hypoxia control; \hat{ } P<0.05 48 hour compared to hypoxia control; & P<0.05 72 hour compared to hypoxia control).

FIG. 13 (FIG. 13) shows that porcine mitochondria treatment of HPAEC decreases brain-derived neurotrophic factor (BDNF) secretion under hypoxic conditions. Porcine mitochondria treatment was maximally effective in reducing BDNF secretion at 3 particles/cell, where BDNF secretion was reduced by 85% compared to untreated hypoxia control HPAEC at 48 hours. Statistical analysis performed was a one-way ANOVA (* P<0.05 24 hour compared to normoxia; # P<0.05 48 hour compared to normoxia; + P<0.05 72 hour compared to normoxia; $ P<0.05 24 hour compared to hypoxia control; \hat{ } P<0.05 48 hour compared to hypoxia control; & P<0.05 72 hour compared to hypoxia control).

FIG. 14 (FIG. 14) shows that porcine mitochondria treatment of HPAEC decreases interleukin-1β (IL-1β) secretion under hypoxic conditions. Porcine mitochondria treatment was maximally effective in reducing IL-1β secretion at 368 particles/cell, where IL-1β secretion was reduced by 70% compared to untreated hypoxia control HPAEC at 48 hours. Statistical analysis performed was a one-way ANOVA (* P<0.05 24 hour compared to normoxia; # P<0.05 48 hour compared to normoxia; +P<0.05 72 hour compared to normoxia; $ P<0.05 24 hour compared to hypoxia control; \hat{ } P<0.05 48 hour compared to hypoxia control; & P<0.05 72 hour compared to hypoxia control).

FIG. 15 (FIG. 15) shows that porcine mitochondria treatment of HPAEC decreases growth/differentiation factor 15 (GDF15) secretion under hypoxic conditions. Porcine mitochondria treatment was maximally effective in reducing GDF15 secretion at 3 particles/cell, where GDF15 secretion was reduced by 70% compared to untreated hypoxia control HPAEC at 48 hours. Statistical analysis performed was a one-way ANOVA (* P<0.05 24 hour compared to normoxia; # P<0.05 48 hour compared to normoxia; + P<0.05 72 hour compared to normoxia; $ P<0.05 24 hour compared to hypoxia control; \hat{ } P<0.05 48 hour compared to hypoxia control; & P<0.05 72 hour compared to hypoxia control).

FIG. 16 (FIG. 16) shows that porcine mitochondria treatment of HPAEC decreases interleukin-6 (IL-6) secretion under hypoxic conditions. Porcine mitochondria treatment was maximally effective in reducing IL-6 secretion at 368 particles/cell, where IL-6 secretion was reduced by 70% compared to untreated hypoxia control HPAEC at 48 hours. Statistical analysis performed was a one-way ANOVA (* P<0.05 24 hour compared to normoxia; # P<0.05 48 hour compared to normoxia; + P<0.05 72 hour compared to normoxia; $ P<0.05 24 hour compared to hypoxia control; \hat{ } P<0.05 48 hour compared to hypoxia control; & P<0.05 72 hour compared to hypoxia control).

FIG. 17 (FIG. 17) shows that porcine mitochondria treatment of HPAEC decreases transforming growth factor-β1 (TGF-β1) secretion under hypoxic conditions. Porcine mitochondria treatment was maximally effective in reducing TGF-β1 secretion at 36 particles/cell, where TGF-β1 secretion was reduced by 95% compared to untreated hypoxia control HPAEC at 48 hours. Statistical analysis performed was a one-way ANOVA (* P<0.05 24 hour compared to normoxia; # P<0.05 48 hour compared to normoxia; + P<0.05 72 hour compared to normoxia; $ P<0.05 24 hour compared to hypoxia control; \hat{ } P<0.05 48 hour compared to hypoxia control; & P<0.05 72 hour compared to hypoxia control).

FIG. 18 (FIG. 18) shows that HPAEC exposed to hypoxic stress take up porcine mitochondria. For the hypoxia recovery group, HPAEC were cultured in normoxia for 24 hours and then in hypoxia (1% O₂) for 24 hours prior to porcine mitochondria treatment. After porcine mitochondria treatment, the hypoxia recovery cells were placed back in normoxia. The hypoxia recovery HPAEC were harvested after 24, 28, or 72 hours of culture in normoxia. For the hypoxia exposure group, HPAEC were cultured in normoxia for 48 hours, treated with porcine mitochondria, and immediately placed in hypoxia (1% O₂). The hypoxia exposure HPAEC were harvested after 24, 28, or 72 hours of hypoxia exposure. As determined using a probe specific for porcine MtND5, HPAEC under hypoxic stress take up the porcine mitochondria in a dose-dependent manner, and maximal expression of porcine MtND5 is achieved at 1,666 particles per cell. In the hypoxia recovery condition, maximal expression of porcine MtND5 is achieved at 48 hours, where a 4,655% increase in porcine mtND5 was observed compared to the untreated hypoxia-recovery control. In the hypoxia exposure condition, maximal expression is achieved at 24 hours, where a 26,680% increase in porcine mtND5 was observed compared to the untreated hypoxia-exposure control. Statistical analysis performed was a one-way ANOVA (* P<0.05 24 hour compared to normoxia; # P<0.05 48 hour compared to normoxia; + P<0.05 72 hour compared to normoxia; $ P<0.05 24 hour compared to hypoxia control; \hat{ } P<0.05 48 hour compared to hypoxia control; & P<0.05 72 hour compared to hypoxia control).

FIG. 19 (FIG. 19) shows that transcription of human mitochondrial DNA in HPAEC exposed to hypoxic stress is largely unaffected by porcine mitochondria treatment. As determined using a probe specific for human MtND5, maximal expression of human MtND5 for both the hypoxia recovery group and the hypoxia exposure group occurs at 72 hours. The time point that appears impacted by porcine mitochondria treatment occurs at 24 hours. In the hypoxia recovery group, there is a trend for decreased human MtND5 expression in HPAEC treated with porcine mitochondria, with 1 particle/cell demonstrating a 33% reduced expression compared to untreated hypoxic controls at 24 hours. In the hypoxia exposure group, there is a trend for increased human MtND5 expression in HPAEC treated with porcine mitochondria, with 1,666 particles/cell resulting in a 36% increase compared to untreated hypoxia-exposure cells at 24 hours. Statistical analysis performed was a one-way ANOVA (* P<0.05 24 hour compared to normoxia; # P<0.05 48 hour compared to normoxia; + P<0.05 72 hour compared to normoxia; $ P<0.05 24 hour compared to hypoxia control; \hat{ } P<0.05 48 hour compared to hypoxia control; & P<0.05 72 hour compared to hypoxia control).

FIG. 20 (FIG. 20) shows that porcine mitochondria treatment of HPAEC reduces TLR-9 expression in hypoxia recovery but increases TLR-9 expression in hypoxia exposure at 24 hours. For both the hypoxia recovery group and the hypoxia exposure group, maximal expression of TLR-9 occurs at 24 hours. In the hypoxia recovery group, there is a trend for decreased TLR-9 expression in HPAEC treated with porcine mitochondria, with 1 particle/cell demonstrating a 38% reduced expression compared to untreated hypoxic controls at 24 hours. In the hypoxia exposure group, there is a trend for increased TLR9 expression in HPAEC treated with porcine mitochondria, with 1,666 particles/cell resulting in a 32% increase compared to untreated hypoxia-exposure cells at 24 hours. Statistical analysis performed was a one-way ANOVA (* P<0.05 24 hour compared to normoxia; # P<0.05 48 hour compared to normoxia; +P<0.05 72 hour compared to normoxia; $ P<0.05 24 hour compared to hypoxia control; \hat{ } P<0.05 48 hour compared to hypoxia control; & P<0.05 72 hour compared to hypoxia control).

FIG. 21 (FIG. 21) shows that porcine mitochondria treatment of HPAEC undergoing hypoxic stress reduces mRNA expression of interleukin-8 (IL-8; CXCL8), IL-6, BH3 interacting-domain death agonist (BID), human MtND1, and human mitochondrial cytochrome B (Mt-CyB). Porcine mitochondria treatment of hypoxic HPAEC is maximally effective for reducing IL-8 expression at 3,687 particles/cell, where a 58% decrease in IL-8 expression was seen compared to untreated hypoxic controls (FIG. 21A). Porcine mitochondria treatment of hypoxic HPAEC is maximally effective for reducing IL-6 expression at 3 particles/cell, where a 30% decrease in IL-6 expression was seen compared to untreated hypoxic controls (FIG. 21B). Porcine mitochondria treatment of hypoxic HPAEC is maximally effective for reducing BID expression at 36 particles/cell, where a 30% decrease in BID expression was seen compared to untreated hypoxic controls (FIG. 21C). Porcine mitochondria treatment of hypoxic HPAEC is maximally effective for reducing human MtND1 expression at 3 particles/cell, where a 57% decrease in MtND1 expression was seen compared to untreated hypoxic controls (FIG. 21D). Porcine mitochondria treatment of hypoxic HPAEC is maximally effective for reducing human Mt-CyB expression at 0.3 particles/cell, where a 57% decrease in MtCyB expression was seen compared to untreated hypoxic controls (FIG. 21E). Statistical analysis performed was a one-way ANOVA (* P<0.05 24 hour compared to normoxia; $ P<0.05 24 hour compared to hypoxia control).

FIG. 22 (FIG. 22) shows that treatment of human endothelial cells with porcine mitochondria decreases hypoxia-induced cell proliferation as indicated by a decrease in total cellular protein content of mitochondria treated HPAEC. HPAEC were treated with 0, 5, 6, or 7 porcine mitochondria per cell and subjected to hypoxic conditions for 24 hours. After the 24-hour exposure to hypoxia, total cellular protein content was measured for each sample via bicinchoninic acid (BCA) assay on HPAEC lysate. Statistical analysis performed was a one-way ANOVA (* P<0.05 compared to control HPAEC not treated with porcine mitochondria).

FIG. 23 (FIG. 23) shows that porcine mitochondria treatment of human alveolar epithelial type II (AT2) cells improved the nucleic acid content of the AT2 cells. AT2 cells were seeded directly from cryo-storage with and without porcine mitochondria and incubated overnight in a standard incubator. Following overnight incubation, the nucleic acid content of AT2 cells treated with porcine mitochondria increased by 23% compared to the untreated AT2 cell control.

FIG. 24 (FIG. 24) shows the mitochondrial activity of isolated porcine mitochondria at various concentrations in respiration buffer containing adenosine diphosphate (ADP).

FIG. 25 (FIG. 25) shows that porcine mitochondria retain mitochondrial activity after cold storage at −80° C. While mitochondria activity decreased at 4° C. over time, storage at −80° C. resulted in retention of approximately 40% OCR (mitochondrial activity). Storage in trehalose improved OCR, resulting in approximately 60% retention in original OCR rate.

FIG. 26 (FIG. 26) shows that porcine mitochondria treatment improves the function of an isolated porcine cadaveric lung while on ex vivo lung perfusion (EVLP). In comparison to the right lung control, isolated porcine mitochondria injected into the left lung increased proliferating cell nuclear antigen (PCNA) positive cells in the lower lung (FIG. 26A), upper lung (FIG. 26B), and mid-lung (FIG. 26C) as measured by histology (FIG. 26A). Porcine mitochondria treatment was maximally effective at 24 hours in the lower lung (FIG. 26A), where a 50% improvement was seen in porcine mitochondria-treated cells compared to control (arrow).

FIG. 27 (FIG. 27) shows that porcine mitochondria treatment improves the parameters of tidal volume (FIG. 27A) and dynamic compression (FIG. 27B) of an isolated porcine cadaveric lung while on EVLP. Isolated porcine mitochondria were injected into an isolated porcine cadaveric lung on EVLP, and perfusion was turned off for 10 minutes while the lung continued inflation. Tidal volume (ml) and dynamic compression (TV/(PIP-PEEP)) were determined at 10 minutes post-injection, 1 hour post-injection, and 4 hours post-injection (TV=tidal volume; PIP=peak inspiratory pressure; PEEP=positive end expiratory pressure). Baseline tidal volume and dynamic compression represent pre-injection tidal volume and dynamic compression, respectively. A 30% improvement in tidal volume and a 40% increase in dynamic compression are seen at 10 minutes post-injection in comparison to baseline.

FIG. 28 (FIG. 28) shows that, following injection of isolated porcine mitochondria into an isolated porcine cadaveric lung on EVLP, there was an immediate and progressive drop in media glucose as well as a 17% decrease in circulating ammonium at one hour post-injection. An isolated porcine cadaveric lung on EVLP was injected with isolated porcine mitochondria 24 minutes after commencement of EVLP and maintained on EVLP for approximately 20 hours. Glucose (g/L) in the circulating media was quantitated using BioPat (FIG. 28A) and Nova (FIG. 28B), and circulating ammonium (NH₄⁺; mmol/L) was quantitated using Nova (FIG. 28C). Initial Nova glucose and ammonium levels represent Nova glucose and ammonium levels at time 0 post-EVLP. Baseline Nova glucose and ammonium levels represent Nova glucose and ammonium levels immediately prior to injection of the porcine mitochondria.

FIG. 29 (FIG. 29) shows that injection of isolated porcine mitochondria into a porcine cadaveric lung on EVLP (“+Mito”) increases tidal volume (mL/kg; FIG. 29A) and gas exchange (ΔPO₂/FiO₂; FIG. 29B) in comparison to a porcine cadaveric lung on EVLP injected with respiration buffer (“Control”).

FIG. 30 (FIG. 30) shows that injection of isolated porcine mitochondria into a porcine cadaveric lung on EVLP (“+ MITO”) decreases the amount of circulating lactate (mg/ml; FIG. 30A), leading to an increased glucose/lactate ratio (FIG. 30B) in comparison to a porcine cadaveric lung on EVLP injected with respiration buffer (“Control”).

FIG. 31 (FIG. 31) shows that injection of isolated porcine mitochondria into a porcine cadaveric lung on EVLP (“+ MITO”) decreases the percentage of apoptotic cells (% TUNEL; FIG. 31A) and increases expression of the cellular adhesion molecule CD31 (FIG. 31B) in comparison to a porcine cadaveric lung injected with respiration buffer (“Control”). The percentage of apoptotic cells was determined by TUNEL assay on tissue biopsies taken from the porcine cadaveric lungs during EVLP. CD31 expression was determined by immunofluorescence staining of tissue biopsies with an anti-CD31 antibody.

FIG. 32 (FIG. 32) shows that the health and function of isolated mitochondria can be rapidly assessed by measuring changes in the size and complexity of mitochondria, mitochondria membrane permeability transition pore (mPTP) opening, or mitochondria respiration. The size and complexity of healthy and damaged mitochondria were measured using flow cytometry. Compared to healthy mitochondria, the damaged mitochondria were larger and less complex, which is indicative of a mitochondrial swelling phenotype (FIG. 32A). mPTP opening was assessed using flow cytometry to measure green fluorescent (FITC) emission of calcein acetoxymethyl (AM)-stained mitochondria. Mitochondria were considered as having a regulated mPTP if they retained calcein-AM, resulting in FITC+ staining. Mitochondria were considered as having dysregulated, continuous mPTP opening if they were unable to retain calcein-AM, resulting in reduced FITC staining. Compared to healthy mitochondria, the damaged mitochondria had drastically reduced FITC emission due to their inability to retain calcein AM (FIG. 32B). To evaluate mitochondria respiration, respiratory control ratios (RCRs) were determined using the Seahorse instrument. RCRs were calculated from the oxygen consumption rate (OCR) during ADP-stimulated respiration (RCR) and uncoupled respiration (RCRmax). The OCR during each of these two states was divided by the basal OCR to obtain the OCR ratio. Maximal respiration was achieved by injecting the mitochondrial protonophore uncoupler BAM15. Compared to healthy mitochondria, the damaged mitochondria had dramatically reduced ADP-stimulated respiration rates and uncoupled respiration rates (FIG. 32C).

FIG. 33 (FIG. 33) shows that the health and function of isolated mitochondria can be rapidly assessed by measuring mitochondria membrane potential or mitochondria membrane permeability. Changes in mitochondria membrane potential were assessed by flow cytometry using a JC-1 assay. Mitochondria depolarization is indicated by a decrease in the red:green fluorescence intensity ratio or by a decrease in the signal intensity in the phycoerythrin (PE) channel. Compared to healthy mitochondria, damaged mitochondria had a decreased red:green ratio and a drastically reduced PE emission (FIG. 33A). Mitochondria permeability was measured by flow cytometry using a SYTOX green nucleic acid stain, which easily permeates mitochondria with compromised membranes. Damaged mitochondria stained with SYTOX green will have higher FITC signal intensity than non-damaged mitochondria stained with SYTOX green. Compared to healthy mitochondria, the damaged mitochondria demonstrated increased FITC emission (FIG. 33B).

FIG. 34 (FIG. 34) shows that mitochondria retain mitochondrial function after cold storage at −80° C., as measured by mitochondria size, complexity, mPTP opening, and respiration. The presence or absence of mitochondrial swelling was assessed using flow cytometry to measure size and complexity of mitochondria stored under non-preserving conditions (i.e., storage at 4° C.) or preserving conditions (i.e., storage at −80° C.). While mitochondria stored at 4° C. almost immediately displayed a swelling phenotype (i.e., increased size, decreased complexity), mitochondria stored at −80° C. retained a normal phenotype comparable to freshly isolated mitochondria throughout the duration of storage (out to 7 months) (FIG. 34A). Mitochondria mPTP opening was assessed using flow cytometry to measure FITC emission of calcein AM-stained mitochondria stored under non-preserving conditions or preserving conditions. Mitochondria were considered as maintaining mPTP if they retained calcein-AM, resulting in FITC+ staining. Mitochondria were considered as failing to maintain mPTP opening if they were unable to retain calcein-AM, resulting in reduced FITC staining. While mitochondria stored at 4° C. lost the ability to regulate their mPTP opening, mitochondria stored at −80° C. controlled mPTP opening comparable to freshly isolated mitochondria throughout the duration of storage (out to 7 months) (FIG. 34B). To evaluate mitochondria respiration of mitochondria stored under non-preserving conditions or preserving conditions, RCRs were determined using the Seahorse instrument. RCRs were calculated from the OCR during ADP-stimulated RCR and uncoupled respiration (RCRmax). The OCR during each of these two states was divided by the basal OCR to obtain the OCR ratio. Maximal respiration was achieved by injecting the mitochondrial protonophore uncoupler BAM15. The ADP-stimulated respiration rates and uncoupled respiration rates of mitochondria stored at 4° C. declined over time, while mitochondria stored at −80° C. had ADP-stimulated respiration rates (FIG. 34C) and uncoupled respiration rates (FIG. 34D) comparable to freshly isolated mitochondria throughout the duration of storage (out to 6 weeks).

FIG. 35 (FIG. 35) shows that mitochondria retain mitochondrial function after cold storage at −80° C., as measured by mitochondria membrane potential and mitochondria membrane permeability. Changes in mitochondria membrane potential of mitochondria stored under non-preserving conditions (i.e., storage at 4° C.) or preserving conditions (i.e., storage at −80° C.) were assessed by flow cytometry using the JC-1 assay. Mitochondria depolarization is indicated by a decrease in the red:green fluorescence intensity ratio or by a decrease in the signal intensity in the phycoerythrin (PE) channel. While mitochondria stored at 4° C. showed a dramatic reduction in membrane potential, mitochondria stored at −80° C. retained membrane potential comparable to freshly isolated mitochondria throughout the duration (out to 7 months) (FIG. 35A). Permeability of mitochondria stored under non-preserving conditions or preserving conditions was measured by flow cytometry using a SYTOX green nucleic acid stain, which easily permeates mitochondria with compromised membranes. Damaged mitochondria stained with SYTOX green will have higher FITC signal intensity than non-damaged mitochondria stained with SYTOX green. While mitochondria stored at 4° C. had an immediate increase in FITC emission, mitochondria stored at −80° C. retained membrane potential comparable to freshly isolated mitochondria through the duration of storage (out to 7 months) (FIG. 35B).

FIG. 36 (FIG. 36) shows that mitochondria retain mitochondrial function after cold storage at −80° C., as measured by their ability to reduce reactive oxygen species (ROS)-mediated chemokine secretion in HPAEC. HPAEC were cultured with 25 μM menadione with or without mitochondria treatment. Mitochondria used in these experiments were stored under either non-preserving conditions (i.e., storage at 4° C.) or preserving conditions (i.e., storage at −80° C.). Chemokines in the culture media of treated HPAEC were measured using bead-based immunoassays. Mitochondria stored at 4° C. rapidly lost their ability to modulate secretion of IL-8/CXCL8 (FIG. 36A), MIG/CXCL9 (FIG. 36B), MCP-1/CCL2 (FIG. 36C), and GROα/CXCL1 (FIG. 36D) compared to mitochondria stored at −80° C., which retained the ability to reduce chemokine secretion.

FIG. 37 (FIG. 37) shows that mitochondria stored at −80° C. have the same gross morphology (FIG. 37A) and average size (FIG. 37B) as freshly isolated mitochondria. Mitochondria scored as class I had a condensed, normal state (i.e., non-damaged state) represented by numerous narrow pleomorphic cristae in a contiguous electron-dense matrix space. Mitochondria scored as class II were in a state of remodeling characterized by reorganized cristae and matrix spaces. The appearance of the remodeling state is temporally correlated with the redistribution and availability of cytochrome c from the intermembrane space. Mitochondria scored as class III were swollen and damaged. Class III mitochondria had intact membranes, but the cristae of these mitochondria have deteriorated and gathered close to the perimeter of the mitochondria. Mitochondria scored as class IV were terminally swollen or ruptured. Class IV mitochondria showed gross morphological derangement, including asymmetric blebbing of matrix. Mitochondria scored as “condensed matrix (CM)” had a condensed matrix with no limiting outer membrane.

FIG. 38 (FIG. 38) shows that intact mitochondria are the functional component in mitochondria treatment as opposed to a component released from the mitochondria after storage at −80° C. or carried over from the isolation process. Mitochondrial and non-mitochondrial fractions were obtained by centrifugation from mitochondria stored for two weeks at −80° C. HPAEC were cultured with 25 μM menadione and treated volumetrically with either the mitochondria fraction or the non-mitochondria fraction. The volumes of 0.02%, 0.2%, 2%, and 20% correspond to 1 mitochondria/cell, 10 mitochondria/cell, 100 mitochondria/cell, and 1,000 mitochondria/cell, respectively. Parameters analyzed included secretion of the inflammatory chemokines IL-8/CXCL8 (FIG. 38A), MCP-1/CCL-2 (FIG. 38B), and GROα/CXCL-1 (FIG. 38C), as well as lactate dehydrogenase (LDH) release (FIG. 38D), which is indicative of cell damage. The mitochondrial fraction alone retained the ability to reduce chemokine secretion and LDH release.

FIG. 39 (FIG. 39) shows that porcine mitochondria treatment improves kidney function and recovery in vivo after acute kidney injury in an ischemia/reperfusion (I/R) mouse model. Acute FR injury was achieved in adult mice by clamping the renal artery for 45 minutes followed by reperfusion. Mice were injected with mitochondria (0.01× or 0.1×) or the vehicle control upon reperfusion on day 1. Blood urea nitrogen (BUN), which is an indicator of kidney function, was increased after I/R injury and trended to decrease at day 2 and on day 4 after mitochondria injection (0.1×) (FIG. 39A). Kidney index, which is the percent mouse weight taken up by the kidney, was increased after FR injury and was reduced after mitochondria injection (0.01×) (FIG. 39B). Kidney injury molecule-1 (KIM1) is a marker of acute kidney injury. While FR injury increased KIM1 serum levels, mitochondria treatment reduced these levels in a dose-responsive manner (FIG. 39C). Monocyte chemoattractant protein 1 (MCP1) is a proinflammatory cytokine associated with acute kidney injury. While FR injury increased MCP1 serum levels, mitochondria treatment reduced these levels in a dose-responsive manner (FIG. 39D). The C3a and C5a members of the compliment system induce inflammatory mediators from both renal tubular epithelial cells and macrophages after hypoxia/reoxygenation. While FR injury increased serum levels of C3a (FIG. 39E) and C5a (FIG. 39F), mitochondria treatment reduced these levels in a dose-dependent manner (FIG. 39E-F). The mitochondria used in these studies were stored for approximately one month at −80° C. prior to injection. Statistical analysis performed was a one-way ANOVA (# P≤0.05 compared to sham; * P≤0.05 compared to model+vehicle).

FIG. 40 (FIG. 40) shows that porcine mitochondria treatment improved the expression of gap junction markers and reduced DNA oxidation in an isolated porcine cadaveric lung placed on EVLP following cold ischemic injury. EVLP was run on isolated porcine cadaveric lungs after approximately 20 hours of cold ischemia time. Mitochondria treatment improved expression of gap junction markers junctional adhesion molecule 1 (JAM1) (FIG. 40A) and CD31 (FIG. 40B) in EVLP after 1 hour in the superior lobe and after 4 hours when measured in the distal segment of the caudal lobe, the proximal segment of the caudal lobe, and the superior lobe. 8-hydroxy-2′-deoxyguanosine (8-OHdG) is a marker of ROS-induced DNA oxidation. Mitochondria treatment decreased expression of 8-OHdG in lung tissue during EVLP after 1 hour in the superior lobe and after 4 hours when measured in the distal segment of the caudal lobe, the proximal segment of the caudal lobe, the inferior lobe, and the superior lobe (FIG. 40C). Protein expression was normalized to DAPI nuclear staining, and all data was normalized to baseline pre-EVLP tissue. Statistical analysis performed was a two-tailed T test.

FIG. 41 (FIG. 41) shows that porcine mitochondria treatment reduced IL-6, IL-8, and interferon (IFN)-γ expression or secretion in isolated porcine cadaveric lungs following cold ischemic injury. EVLP was run on isolated porcine cadaveric lungs after approximately 20 hours of cold ischemia time. Mitochondria treatment decreased circulating IL-6 during EVLP (FIG. 41A) and decreased lung tissue lysate levels of IL-8 after 1 hour EVLP in the superior lobe and after 4 hours EVLP in the distal segment of the caudal lobe, the proximal segment of the caudal lobe, and the superior lobe (FIG. 41B).

FIG. 42 (FIG. 42) shows the effect of mitochondria injection on pulmonary vascular resistance (PVR) during EVLP. PVR of isolated porcine cadaveric lungs was measured during EVLP. Six lungs (“Control”) were treated with vehicle at the EVLP time of 3 hours, and five lungs were treated with mitochondria (“Mitochondria”) at the EVLP time of 3 hours were included in the analysis (FIG. 42A). A single mitochondria-treated lung is shown in FIG. 42B to demonstrate how mitochondria injection can be visually seen at the 3-hour injection. The dotted lines in FIG. 42A and FIG. 42B represent the time of mitochondrial injection. The arrows in FIG. 42B represent the times at which gas exchange was assessed. Between each assessment was a recruitment event. Statistical analysis performed was a one-way ANOVA (#P≤0.01 compared to control; *P≤0.05 compared to control).

FIG. 43 (FIG. 43) shows the pathways impacted by mitochondria treatment of isolated porcine cadaveric lungs placed on EVLP following cold ischemic injury. Isolated porcine cadaveric lungs were exposed to approximately 20 hours of cold ischemia time, after which EVLP was run on the lungs for 5 hours. Distal caudal and proximal caudal lung tissue was collected from control buffer injected or mitochondrial injected lungs and subjected to RNA sequencing. Relative to control samples, mitochondria treatment decreased inflammatory and apoptotic pathways.

FIG. 44 (FIG. 44) shows that mitochondria treatment reduces ROS-mediated oxidative byproducts and ROS-mediated chemokine secretion. HPAEC were cultured with 25 μM of the ROS-inducer menadione with or without mitochondria treatment for 5 hours. The oxidative stress markers 4-hydroxynonenal (4-HNE) and 8-OHdG were measured in lysates of the treated cells by competitive ELISA. Mitochondria treatment effectively reduced levels of 4-HNE adducts (FIG. 44A) and 8-OHdG (FIG. 44B) to normal (no menadione treatment) levels. Cell culture supernatants of the treated cells were analyzed for the presence of secreted chemokines by flow cytometry. Mitochondria treatment effectively reduced secretion of IL-8/CXCL8 (FIG. 44C), MCP1/CCL2 (FIG. 44D), MIG/CXCL9 (FIG. 44E), and GROα/CXCL1 to normal (no menadione treatment) levels. The mitochondria used for these experiments were stored at −80° C. for 1 week prior to use. Statistical analysis performed was a one-way ANOVA (***P<0.0001 compared to 25 μM menadione untreated; ****P<0.0001 compared to 25 μM menadione untreated).

FIG. 45 (FIG. 45) shows that mitochondria treatment reduces ROS-mediated damage and improves viability of HPAEC subjected to cold/rewarming injury. To replicate cold/rewarming injury in a two-dimensional (2D) culture model, HPAEC were cultured at 4° C. for 24 hours (hypothermic conditions) and rewarmed at 37° C. for 4 hours (normothermic conditions), as shown in FIG. 45A. The treatment groups included HPAEC treated with mitochondria at the onset of hypothermia and HPAEC treated with mitochondria at rewarming. After the 4-hour rewarming period, ROS-mediated damage was measured using a 4-HNE adduct competitive ELISA for quantitation of 4-HNE protein adducts in HPAEC lysates. 4-HNE adduct formation was very sensitive to mitochondria treatment as very low doses of mitochondria were able to have an impact (FIG. 45B). Cellular viability was also measured after the 4-hour rewarming period. Results are shown in FIG. 45C as relative light units (RLU) normalized to baseline (i.e., HPAEC exposed to cold/rewarming with no mitochondria treatment). Normal, unstressed HPAEC are represented by a dashed line (FIG. 45C). Mitochondria treatment produced a 2-3 fold increase in cellular viability compared to untreated HPAEC (FIG. 45C).

FIG. 46 (FIG. 46) shows that mitochondria treatment reduces necrosis of HPAEC subjected to cold/rewarming injury. Cold/rewarming injury was replicated using the 2D culture method shown in FIG. 45A. The treatment groups included HPAEC treated with mitochondria at the onset of hypothermia and HPAEC treated with mitochondria at rewarming. After the 4-hour rewarming period, necrotic cell death was measured using a cell-impermeant, profluorescent DNA dye. Results are shown in FIG. 46A as relative light units (RLU) normalized to baseline (i.e., HPAEC exposed to cold/rewarming with no mitochondria treatment). HPAEC treated with mitochondria showed a dose-dependent decrease in necrosis (FIG. 46A). A hallmark of necrotic cell death is the phosphorylation of Mixed Lineage Kinase Domain Like Pseudokinase (MLKL). HPAEC lysates collected after the 4-hour warming period were analyzed using a sandwich ELISA to measure phospho-MLKL (pMLKL) and total MLKL. Results are shown in FIG. 46B as optical density measured at a wavelength of 450 nm (OD₄₅₀) normalized to baseline (i.e., HPAEC exposed to cold/rewarming with no mitochondria treatment). HPAEC treated with mitochondria showed a dose-dependent decrease in pMLKL levels (FIG. 46B). Total MLKL levels were unchanged (data not shown). High Mobility Group Box 1 (HMGB-1) is a ubiquitous nuclear protein passively released by cells undergoing necrosis. Released HMGB-1 in HPAEC culture supernatants was measured by sandwich ELISA. The results shown in FIG. 46C were normalized to baseline (i.e., HPAEC exposed to cold/rewarming with no mitochondria treatment). Mitochondria treatment reduced HMGB-1 release compared to untreated cells (FIG. 46C). Lactate dehydrogenase (LDH) is a stable cytosolic enzyme that is released upon cell lysis. Released LDH in HPAEC culture supernatants was measured with a 30-minute coupled enzymatic assay, which results in conversion of a tetrazolium salt (INT) into a red formazan product. Results are shown in FIG. 46D as optical density measured at a wavelength of 490 nm (OD₄₉₀) normalized to baseline (i.e., HPAEC exposed to cold/rewarming with no mitochondria treatment). Mitochondria treatment reduced LDH release compared to untreated cells (FIG. 46D). Normal, unstressed HPAEC controls are represented in FIGS. 46A, 46B, and 46D by a dashed line.

FIG. 47 (FIG. 47) shows that mitochondria treatment increases total levels of cellular ATP in HPAEC subjected to cold/rewarming injury, which correlates with improved cell viability. Cold/rewarming injury was replicated using the 2D culture method shown in FIG. 45A. The treatment groups included HPAEC treated with mitochondria at the onset of hypothermia and HPAEC treated with mitochondria at rewarming. After the 4-hour rewarming period, total levels of cellular ATP were measured using a luminescent ATP detection assay. The results shown in FIG. 47A were normalized to baseline (i.e., HPAEC exposed to cold/rewarming with no mitochondria treatment). Mitochondria treated HPAEC had increased ATP concentrations compared to untreated cells. There is a positive correlation between increased ATP concentration and cell viability (FIG. 47B) and a negative correlation between increased ATP concentration and necrosis (FIG. 47C). Statistical analysis performed was a one-way ANOVA.

FIG. 48 (FIG. 48) shows that mitochondria treatment improves cell viability and reduces necrosis in lung homogenates. After 24 hours in cold storage, distal pieces of lung were collected, enzymatically digested, and placed into normothermic (rewarming) cell culture conditions. Mitochondria treatments (500 particles/mg or 1,000 particles/mg) were based on wet tissue weight. Compared to untreated lung homogenates, mitochondria treatment significantly improved cell viability (FIG. 48A) and reduced necrosis (FIG. 48B). Statistical analysis performed was a one-way ANOVA (****P<0.0001 compared to untreated).

FIG. 49 (FIG. 49) shows that mitochondria treatment reduces IL-6 and IFN-γ secretion by lung homogenates. After overnight storage at 4° C., lung tissue was homogenized, treated with increasing doses of mitochondria, and incubated at standard culture conditions (37° C.) overnight. IL-6 and IFN-γ were measured in the lung homogenate lysates after the overnight culture under standard conditions. Mitochondria treatment decreased secretion of IL-6 and IFN-γ compared to untreated control lung homogenates. Statistical analysis performed was a one-way ANOVA (*P<0.05 compared to INF-γ control; #P<0.05 compared to IL-6 control).

### DETAILED DESCRIPTION OF THE INVENTION

The present invention will be now illustrated by the following examples without limiting the scope of said invention.

### I. Definitions

To facilitate an understanding of the present invention, a number of terms and phrases are defined below. Unless otherwise noted, technical terms are used according to conventional usage.

As used herein, the terms “about” and “approximately,” when used to modify a numeric value or numeric range, indicate the deviations of 5% to 10% above and 5% to 10% below the value or range remain within the intended meaning of the recited value or range.

“Administering” (or any form of administration such as “administered”) means delivery of an effective amount of composition to a subject as described herein. Exemplary routes of administration include, but are not limited to, injection (such as subcutaneous, intramuscular, intradermal, and intravenous), oral, dermal, and transdermal routes.

The terms “anoxia,” “anoxic,” and “anoxic conditions” may refer to conditions under which the supply of oxygen to an organ, tissue, or cell is cut off. The terms “anoxia,” “anoxic,” and “anoxic conditions” may also refer to a virtually complete absence of oxygen in the organ, tissue, or cell, which, if prolonged, may result in death of the organ, tissue, or cell.

The term “detection,” as used herein, refers to quantitatively or qualitatively identifying a nucleotide, nucleic acid, or protein within a sample.

The term “differentiation” refers to any process by which an unspecialized (“uncommitted”) or less specialized cell acquires the features of a specialized cell, such as a nerve cell, muscle cell, or macrophage, for example. A differentiated cell is one that has taken on a more specialized (“committed”) position within the lineage of a cell. The term committed, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type.

The terms “exogenous” and “heterologous” are used interchangeably herein and include a nucleic acid, protein, or organelle (e.g., porcine mitochondria) that is not normally present in a prokaryotic or eukaryotic cell. These terms, when used with reference to portions of a nucleic acid, indicate that the nucleic acid comprises two or more subsequences that are not found in the same relationship to each other in nature. For instance, the nucleic acid is typically recombinantly produced, having two or more sequences from unrelated genes arranged to make a new functional nucleic acid (e.g., a promoter from one source and a coding region from another source). Similarly, a heterologous protein indicates that the protein comprises two or more subsequences that are not found in the same relationship to each other in nature (e.g., a fusion protein).

The term “ex vivo” refers to a condition applied to a cell, a tissue, or other sample obtained from an organism that takes place outside the organism.

As used herein, the terms “freeze-thaw” and “freeze-thaw cycle” refer to freezing of the mitochondria of the invention to a temperature below 0° C., maintaining the mitochondria in a temperature below 0° C. for a defined period of time and thawing the mitochondria to room temperature or body temperature or any temperature above 0° C. which allows administering the mitochondria according to the methods of the invention. Each possibility represents a separate embodiment of the present invention. The term “room temperature,” as used herein refers to a temperature of between 18° C. and 25° C. In another embodiment, the mitochondria that have undergone a freeze-thaw cycle were frozen at a temperature of at least −70° C. In another embodiment, the mitochondria that have undergone a freeze-thaw cycle were frozen at a temperature of at least −20° C. In another embodiment, the mitochondria that have undergone a freeze-thaw cycle were frozen at a temperature of at least −4° C. In another embodiment, the mitochondria that have undergone a freeze-thaw cycle were frozen at a temperature of at least 0° C. According to another embodiment, freezing of the mitochondria is gradual. According to some embodiment, freezing of mitochondria is through flash-freezing. As used herein, the term “flash-freezing” refers to rapidly freezing the mitochondria by subjecting them to cryogenic temperatures.

According to another embodiment, the mitochondria are frozen in freezing buffer comprising a cryoprotectant. According to some embodiments, the cryoprotectant is a lipid, a protein, a saccharide, a disaccharide, an oligosaccharide a polysaccharide, or any combination thereof. In preferred embodiments, the cryoprotectant is trehalose, sucrose, glycerol, plasmaLyte, CryoStor, dimethyl sulfoxide (DMSO), glutamate, albumin, polyethylene glycols (PEGs), poly(vinyl alcohols) (PVAs), or any combination thereof. Each possibility represents a separate embodiment of the present invention. According to another embodiment, the cryoprotectant concentration in the freezing buffer is a sufficient cryoprotectant concentration which acts to preserve mitochondrial function. Without wishing to be bound by any theory or mechanism, mitochondria that have been frozen within a freezing buffer comprising a saccharide, a disaccharide (e.g., sucrose, trehalose), an oligosaccharide, or a polysaccharide demonstrate a comparable or higher oxygen consumption rate following thawing, as compared to control mitochondria that have not undergone a freeze-thaw cycle or that have been frozen within a freezing buffer or isolation buffer without a saccharide, a disaccharide (e.g., sucrose, trehalose), an oligosaccharide, or a polysaccharide.

According to some embodiments, the term “functional mitochondria” refers to mitochondria that consume oxygen. According to another embodiment, functional mitochondria have an intact outer membrane. According to some embodiments, functional mitochondria are intact mitochondria. In another embodiment, functional mitochondria consume oxygen at an increasing rate over time. In another embodiment, the functionality of mitochondria is measured by oxygen consumption. In another embodiment, oxygen consumption of mitochondria may be measured by any method known in the art such as, but not limited to, the MitoXpress fluorescence probe (Luxcel) and Seahorse assay. According to some embodiments, functional mitochondria are mitochondria which display an increase in the rate of oxygen consumption in the presence of ADP and a substrate such as, but not limited to, glutamate, malate or succinate. Each possibility represents a separate embodiment of the present invention. In another embodiment, functional mitochondria are mitochondria which produce ATP. In another embodiment, functional mitochondria are mitochondria capable of manufacturing their own RNAs and proteins and are self-reproducing structures. In another embodiment, functional mitochondria produce a mitochondrial ribosome and mitochondrial tRNA molecules.

The term “gene” refers to a nucleic acid that encodes an RNA, for example, nucleic acid sequences including, but not limited to, a structural gene encoding a polypeptide.

The terms “hypoxia,” “hypoxic,” and “hypoxic conditions” refer to a condition under which an organ, tissue, or cell receive an inadequate supply of oxygen.

As used herein, the term “intact mitochondria” refers to mitochondria comprising an outer and an inner membrane, an inter-membrane space, the cristae (formed by the inner membrane) and the matrix. In another embodiment, intact mitochondria comprise mitochondrial DNA. In another embodiment, intact mitochondria contain active respiratory chain complexes I-V embedded in the inner membrane. In another embodiment, intact mitochondria consume oxygen. According to another embodiment, intactness of a mitochondrial membrane may be determined by any method known in the art. In a non-limiting example, intactness of a mitochondrial membrane is measured using the tetramethylrhodamine methyl ester (TMRM) or the tetramethylrhodamine ethyl ester (TMRE) fluorescent probes. Each possibility represents a separate embodiment of the present invention. Mitochondria that were observed under a microscope and show bright TMRM or TMRE staining have an intact mitochondrial outer membrane.

The term “ischemia” is defined as an insufficient supply of blood to a specific organ, tissue, or cell. A consequence of decreased blood supply is an inadequate supply of oxygen to the organ, tissue, or cell (hypoxia). Prolonged hypoxia may result in injury to the affected organ, tissue, or cell.

A polypeptide, antibody, polynucleotide, vector, cell, or composition which is “isolated” is a polypeptide, polynucleotide, vector, cell, or composition which is in a form not found in nature. Isolated polypeptides, polynucleotides, vectors, cells, or compositions include those that have been purified to a degree that they are no longer in a form in which they are found in nature. In some embodiments, a polypeptide, polynucleotide, vector, cell, or composition which is isolated is substantially pure.

As used herein, the term “isolated mitochondria” refers to mitochondria separated from other cellular components, wherein the weight of the mitochondria constitutes more than 80% of the combined weight of the mitochondria and other sub-cellular fractions. Preparation of isolated mitochondria may involve changing buffer composition or additional washing steps, cleaning cycles, centrifugation cycles and sonication cycles which are not required in preparation of partially purified mitochondria. Without wishing to be bound by any theory or mechanism, such additional steps and cycles may harm the functionality of the isolated mitochondria. As used herein, mitochondria of a xenogeneic source refer to mitochondria derived from a different subject than the subject to be treated from a different species. As used herein, mitochondria of an autologous source refer to mitochondria derived from the same subject to be treated. As used herein, mitochondria of an allogeneic source refer to mitochondria derived from a different subject than the subject to be treated from the same species.

As used herein, the term “mitochondrial membrane” refers to a mitochondrial membrane selected from the mitochondrial inner membrane, the mitochondrial outer membrane or a combination thereof.

As used herein, the term “mitochondrial proteins” refers to proteins which originate from mitochondria, including mitochondrial proteins which are encoded by genomic DNA or mtDNA. As used herein, the term “cellular proteins” refers to all proteins which originate from the cells or tissue from which the mitochondria are produced.

The term “modulate” or “modulates” means that gene expression or level of RNA molecule or equivalent RNA molecules encoding one or more protein or protein subunits or peptides, or activity of one or more protein subunits or peptides, is up-regulated or down-regulated such that the expression, level, or activity is greater than or less than that observed in the absence of the modulator. The term “modulate” includes “inhibit.”

As used herein, the terms “normoxic” and “normoxia” refer to a state of normal levels of oxygen.

The terms “nucleotide sequences” and “nucleic acid sequences” refer to deoxyribonucleic acid (DNA) or ribonucleic acid (RNA) sequences, including, without limitation, messenger RNA (mRNA), DNA/RNA hybrids, or synthetic nucleic acids. The nucleic acid may be single-stranded, or partially or completely double stranded (duplex). Duplex nucleic acids may be homoduplex or heteroduplex.

As used herein, the term “organ” refers to a part or structure of the body, which is adapted for a special function or functions. In a particular embodiment, the organ is the lungs, the liver, the kidneys, the heart, the pancreas and the bowel, including the stomach and intestines.

The term “pharmaceutically acceptable carrier or excipient”, which may be used interchangeably with the term biologically compatible carrier or excipient, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Pharmaceutically acceptable carriers or excipients suitable for use in the present invention include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

As used herein, the term “polynucleotide” refers to a polymer of ribonucleic acid (RNA) or deoxyribonucleic acid (DNA). A polynucleotide is made up of four bases: adenine, cytosine, guanine, and thymine/uracil (uracil is used for RNA). A coding sequence from a nucleic acid is indicative of the sequence of the protein encoded by the nucleic acid. The term includes various modifications and analogues known in the art.

The terms “protein,” “peptide,” “polypeptide,” and “amino acid sequence” are used interchangeably herein to refer to polymers of amino acid residues of any length. The polymers may be linear or branched. The polymers may comprise modified amino acids or amino acid analogs and may be interrupted by chemical moieties other than amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling or bioactive component.

The term “recombinant” with reference to a nucleic acid or polypeptide refers to one that has a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques. A recombinant polypeptide may also refer to a polypeptide that has been made using recombinant nucleic acids, including recombinant nucleic acids transferred to a host organism that is not the natural source of the polypeptide. The term “recombinant” when used with reference to a cell, virus, or vector indicates that the cell, virus, or vector has been modified by or is the result of laboratory methods. A recombinant cell, virus, or vector can include a cell, virus, or vector that has been modified by the introduction of a heterologous nucleic acid or protein or the alteration of a native nucleic acid or protein. Thus, for example, recombinant cells include cells that express genes that are not found within the native (non-recombinant) form of the cell or express native genes that are otherwise abnormally expressed, under expressed, or not expressed at all.

The term “reperfusion” refers to the resumption of blood flow in a tissue or organ following a period of ischemia.

The term “sample” is used in its broadest sense. A sample suspected of containing a nucleic acid can comprise a cell, chromosomes isolated from a cell (e.g., a spread of metaphase chromosomes), genomic DNA, RNA, cDNA and the like.

As used herein, the terms “stem cell” and “progenitor cells” refers to a cell capable of self-replication and pluripotency. Typically, stem cells and progenitor cells can regenerate an injured tissue. Stem cells and progenitor cells herein may be, but are not limited to, embryonic stem (ES) cells or tissue stem cells (also called tissue-specific stem cell, or somatic stem cell). Any artificially produced cell which can have the above-described abilities (e.g., fusion cells, reprogrammed cells, or the like used herein) may be a stem cell or progenitor cell. ES cells are pluripotent stem cells derived from early embryos.

As used herein, the term “subject” includes any human or nonhuman animal. The term “nonhuman animal” includes, but is not limited to, vertebrates such as nonhuman primates, sheep, dogs, cats, rabbits, ferrets, rodents (such as mice, rats and guinea pigs), avian species (such as chickens), amphibians, and reptiles. In preferred embodiments, the subject is a mammal such as a nonhuman primate, sheep, dog, cat, rabbit, ferret, or rodent. In more preferred embodiments, the subject is a human. The terms “subject,” “patient,” and “individual” are used interchangeably herein.

The terms “transfection,” “transduction,” “transfecting,” or “transducing,” can be used interchangeably and are defined as a process of introducing a nucleic acid molecule or a protein into a cell. Nucleic acids are introduced into a cell using non-viral or viral-based methods. The nucleic acid molecule can be a sequence encoding complete proteins or functional portions thereof. Typically, a nucleic acid vector, comprising the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. For viral-based methods, any useful viral vector can be used in the methods described herein. Examples of viral vectors include, but are not limited to retroviral, adenoviral, lentiviral, and adeno-associated viral vectors. In some aspects, the nucleic acid molecules are introduced into a cell using an adenoviral vector following standard procedures known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford, K. G., et al., Gene Ther. 2001 January; 8(1): 1-4 and Prochiantz, A., Nat Methods. 2007 February; 4(2): 119-20.

As used herein, terms such as “treating,” “treatment,” “treat,” or “to treat” refer to an intervention or a therapeutic measure that ameliorates a sign or symptom of disease, pathological condition, or disorder. As used herein, the terms “treating,” “treatment,” “treat,” and “to treat,” with reference to a disease, disorder, pathological condition or symptom, also refers to any observable beneficial effect of the treatment. The beneficial effect may be evidenced, for example, by: a delayed onset of symptoms of the disease, condition, or disorder; a slower progression of the disease, condition, or disorder; a reduction in the number of relapses of the disease, condition, or disorder; an improvement in the overall health or well-being of the subject; or by other parameters known in the art that are specific to the particular disease, condition, or disorder. A prophylactic treatment is a treatment administered to a subject who does not exhibit signs of a disease, condition, or disorder or exhibits only early signs, for the purpose of decreasing the risk of developing pathology. A therapeutic treatment is a treatment administered to a subject after signs and symptoms of the disease, condition, or disorder have developed.

The term “vector” means a construct which is capable of delivering and expressing one or more genes or sequences of interest in a host cell. Examples of vectors include, but are not limited to, viral vectors, naked DNA or RNA expression vectors, plasmid vectors, cosmid vectors, phage vectors, DNA or RNA expression vectors associated with cationic condensing agents, DNA or RNA expression vectors encapsulated in liposomes, and certain eukaryotic cells, such as producer cells.

As used in the present disclosure and claims, the singular forms “a,” “an,” and “the” include the plural forms unless the context clearly dictates otherwise.

The terms “comprising,” “including,” “having,” and the like, as used with respect to embodiments, are synonymous. It is understood that wherever embodiments described herein with the language “comprising,” otherwise analogous embodiments described in terms of “consisting of” and/or “consisting essentially of” are also provided.

For the purpose of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B, and C).

The description may use the terms “embodiment” or “embodiments,” which may each refer to one or more of the same or different embodiments.

### II. Methods of Organ Transplantation

Disclosed herein is a method of organ transplantation, the method comprising delivering isolated mitochondria to an organ intended for transplantation. In some embodiments, the organ is from a human donor, allogeneic, heterogeneic, from a non-human donor (e.g., porcine), or engineered whether entirely or partially (e.g., a decellularized matrix from a porcine kidney recellularized for transplantation). In some embodiments, the method further comprises harvesting the organ from a donor. In some embodiments, the method further comprises transplanting the organ treated with the isolated mitochondria into a recipient. In some embodiments, the isolated mitochondria are isolated human mitochondria allogeneic to the recipient. In some embodiments, the isolated mitochondria are isolated mitochondria autologous to the recipient. In preferred embodiments, the organ intended for transplantation is harvested from a human donor. In some embodiments, the isolated mitochondria are isolated human mitochondria allogeneic to the human donor. In some embodiments, the isolated mitochondria are isolated human mitochondria autologous to the human donor. In preferred embodiments, the organ intended for transplantation is engineered from a porcine organ scaffold. In some embodiments, the isolated mitochondria are isolated porcine mitochondria.

In preferred embodiments, the cells of the organ treated with the isolated mitochondria have at least 1%, or at least 2%, or at least 5%, or at least 10%, or at least 20%, or at least 50%, or at least 100% improvement in mitochondrial function in comparison to cells of a corresponding organ not treated with the isolated mitochondria. In some embodiments, the isolated mitochondria are delivered to the organ prior to the step of harvesting the organ from the donor. In other embodiments, the isolated mitochondria are delivered to the organ after the step of harvesting the organ from the donor. In preferred embodiments, the organ is a human organ. In other embodiments, the organ is a pig organ for xenotransplantation into the recipient.

In preferred embodiments, the organ is a lung. In particularly preferred embodiments, the lung treated with the isolated mitochondria is transplanted into a human recipient suffering from pulmonary hypertension. In particularly preferred embodiments, the lung is a human lung. In some embodiments, the isolated mitochondria are delivered to the lung through the airway, intravenously, or intra-arterially.

In preferred embodiments, the organ is a kidney. In particularly preferred embodiments, the kidney treated with the isolated mitochondria is transplanted into a human recipient suffering from a kidney disease or disorder. In particularly preferred embodiments, the kidney is a human kidney. In some embodiments, the isolated mitochondria are delivered to the kidney intravenously or intra-arterially.

In some embodiments, the organ, kidney, or lung treated with the isolated mitochondria has reduced inflammation and/or immune cell activation in comparison to a corresponding organ, kidney, or lung not treated with the isolated mitochondria. In preferred embodiments, the reduced inflammation and/or immune cell activation is associated with reduced expression of MAPK14, JNK, or p53, by at least 1%, or at least 2%, or at least 5%, or at least 10%, or at least 20%, or at least 50%, or at least 80%. In preferred embodiments, the reduced inflammation and/or immune cell activation is associated with reduced expression of NF-κB, by at least 1%, or at least 2%, or at least 5%, or at least 10%, or at least 20%, or at least 50%, or at least 80%. In preferred embodiments, the reduced inflammation and/or immune cell activation is associated with reduced secretion of pro-inflammatory cytokines and chemokines such as MIP-1β (CCL4), PDGF-BB, RANTES (CCL5), soluble ICAM-1 (sICAM-1), M-CSF (CSF-1), IL-1β, IL-6, IL-8 (CXCL8), GDF-15, TGF-β1, and any combination thereof, by at least 1%, or at least 2%, or at least 5%, or at least 10%, or at least 20%, or at least 50%, or at least 80%. In preferred embodiments, the reduced inflammation and/or immune cell activation is associated with reduced expression of activation markers such as CD69, CD95, CD30, CD137, CD25 (IL2RA), CD38, CD154 (CD40L), and any combination thereof, by at least 1%, or at least 2%, or at least 5%, or at least 10%, or at least 20%, or at least 50%, or at least 80%. In preferred embodiments, the reduced inflammation and/or immune cell activation is associated with reduced expression or secretion of IL-2, IL-4, IL-5, IL-6, IL-9, IL-13, IL17, TNF-α, IFN-γ, or any combination thereof, by at least 1%, or at least 2%, or at least 5%, or at least 10%, or at least 20%, or at least 50%, or at least 80%.

In some embodiments, the organ, kidney, or lung treated with the isolated mitochondria has reduced cellular apoptosis, increased cell viability, reduced mitochondrial stress signaling, and/or reduced cell damage in comparison to a corresponding organ, kidney, or lung not treated with the isolated mitochondria. In preferred embodiments, the reduced cell damage is associated with reduced TLR9 expression, altered heme oxygenase-1 (HO-1) expression, reduced cytosolic mtDNA, or any combination thereof, by at least 1%, or at least 2%, or at least 5%, or at least 10%, or at least 20%, or at least 50%, or at least 80%. In some embodiments, the altered HO-1 expression is increased HO-1 expression after cold exposure. In preferred embodiments, the reduced cellular apoptosis, increased cell viability, reduced mitochondrial stress signaling, and/or reduced cell damage is associated with reduced NF-κB, MAPK14, JNK, p53 expression, or any combination thereof, by at least 1%, or at least 2%, or at least 5%, or at least 10%, or at least 20%, or at least 50%, or at least 80%. In preferred embodiments, the reduced cellular apoptosis is associated with reduced pro-apoptotic marker expression, by at least 1%, or at least 2%, or at least 5%, or at least 10%, or at least 20%, or at least 50%, or at least 80%. In particularly preferred embodiments, the reduced cellular apoptosis is associated with reduced expression of Bax, Bid, Bad, or any combination thereof, by at least 1%, or at least 2%, or at least 5%, or at least 10%, or at least 20%, or at least 50%, or at least 80%. In preferred embodiments, the reduced cellular apoptosis is associated with increased anti-apoptotic marker expression, by at least 1%, or at least 2%, or at least 5%, or at least 10%, or at least 20%, or at least 50%, or at least 80%. In particularly preferred embodiments, the reduced cellular apoptosis is associated with increased expression of Bcl-2 and/or Mcl-1 by at least 1%, or at least 2%, or at least 5%, or at least 10%, or at least 20%, or at least 50%, or at least 80%.

In some embodiments, the organ, kidney, or lung treated with the isolated mitochondria has increased glucose uptake and decreased lactate production in comparison to a corresponding organ, kidney, or lung not treated with the isolated mitochondria. In preferred embodiments, the increased glucose uptake and decreased lactate production is associated with increased expression of HK, GLUT, VDAC1, AKT1, or any combination thereof, by at least 1%, or at least 2%, or at least 5%, or at least 10%, or at least 20%, or at least 50%, or at least 80%.

Also disclosed herein is a method of improving the performance of an implanted tissue or transplanted organ in a subject, the method comprising delivering isolated mitochondria to a tissue or organ before, during, or after implantation or transplantation of the tissue or organ, wherein the tissue or organ is a donor tissue, donor organ, engineered tissue, or engineered organ. In some embodiments, the isolated mitochondria are isolated porcine mitochondria. In some embodiments, the isolated mitochondria are isolated human mitochondria allogeneic to the tissue or organ. In some embodiments, the isolated mitochondria are isolated human mitochondria autologous to the tissue or organ. In preferred embodiments, the tissue or organ is a human tissue or organ. In other embodiments, the tissue or organ is a pig tissue or organ for xenotransplantation into the subject. In preferred embodiments, the organ is a kidney. In preferred embodiments, the organ is a lung. In particularly preferred embodiments, the lung is a human lung. In some embodiments, the isolated mitochondria are delivered to the lung through the airway, intravenously, or intra-arterially. In preferred embodiments, the tissue or organ is selected from the group consisting of: blood vessels, ureter, trachea, and skin patch. In preferred embodiments, the organ is a kidney. In particularly preferred embodiments, the kidney is a human kidney. In some embodiments, the isolated mitochondria are delivered to the kidney intravenously or intra-arterially.

In preferred embodiments, the cells of the tissue or organ treated with the isolated mitochondria have at least 1%, or at least 2%, or at least 5%, or at least 10%, or at least 20%, or at least 50%, or at least 100% improvement in mitochondrial function in comparison to cells of a corresponding organ or tissue not treated with the isolated mitochondria.

In some embodiments, the tissue or organ treated with the isolated mitochondria has reduced inflammation and/or immune cell activation in comparison to a corresponding tissue or organ not treated with the isolated mitochondria. In preferred embodiments, the reduced inflammation and/or immune cell activation is associated with reduced expression of MAPK14, JNK, or p53, by at least 1%, or at least 2%, or at least 5%, or at least 10%, or at least 20%, or at least 50%, or at least 80%. In preferred embodiments, the reduced inflammation and/or immune cell activation is associated with reduced expression of NF-κB, by at least 1%, or at least 2%, or at least 5%, or at least 10%, or at least 20%, or at least 50%, or at least 80%. In preferred embodiments, the reduced inflammation and/or immune cell activation is associated with reduced secretion of pro-inflammatory cytokines and chemokines such as MIP-1β (CCL4), PDGF-BB, RANTES (CCL5), soluble ICAM-1 (sICAM-1), M-CSF (CSF-1), IL-1β, IL-6, IL-8 (CXCL8), GDF-15, TGF-β1, and any combination thereof, by at least 1%, or at least 2%, or at least 5%, or at least 10%, or at least 20%, or at least 50%, or at least 80%. In preferred embodiments, the reduced inflammation and/or immune cell activation is associated with reduced expression of activation markers such as CD69, CD95, CD30, CD137, CD25 (IL2RA), CD38, CD154 (CD40L), and any combination thereof, by at least 1%, or at least 2%, or at least 5%, or at least 10%, or at least 20%, or at least 50%, or at least 80%. In preferred embodiments, the reduced inflammation and/or immune cell activation is associated with reduced expression or secretion of IL-2, IL-4, IL-5, IL-6, IL-9, IL-13, IL17, TNF-α, IFN-γ, or any combination thereof, by at least 1%, or at least 2%, or at least 5%, or at least 10%, or at least 20%, or at least 50%, or at least 80%.

In some embodiments, the tissue or organ treated with the isolated mitochondria has reduced cellular apoptosis, increased cell viability, reduced mitochondrial stress signaling, and/or reduced cell damage in comparison to a corresponding tissue or organ not treated with the isolated mitochondria. In preferred embodiments, the reduced cell damage is associated with reduced TLR9 expression, altered HO-1 expression, reduced cytosolic mtDNA, or any combination thereof, by at least 1%, or at least 2%, or at least 5%, or at least 10%, or at least 20%, or at least 50%, or at least 80%. In some embodiments, the altered HO-1 expression is increased HO-1 expression after cold exposure. In preferred embodiments, the reduced cellular apoptosis, increased cell viability, reduced mitochondrial stress signaling, and/or reduced cell damage is associated with reduced NF-κB, MAPK14, JNK, p53 expression, or any combination thereof, by at least 1%, or at least 2%, or at least 5%, or at least 10%, or at least 20%, or at least 50%, or at least 80%. In preferred embodiments, the reduced cellular apoptosis is associated with reduced pro-apoptotic marker expression, by at least 1%, or at least 2%, or at least 5%, or at least 10%, or at least 20%, or at least 50%, or at least 80%. In particularly preferred embodiments, the reduced cellular apoptosis is associated with reduced expression of pro-apoptotic initiators (BIM, PUMA), pro-apoptotic effectors (BAX, BAK), apoptogenic factors (SMAC, DIABLO, BID, BAD, etc.) or any combination thereof, by at least 1%, or at least 2%, or at least 5%, or at least 10%, or at least 20%, or at least 50%, or at least 80%. In preferred embodiments, the reduced cellular apoptosis is associated with increased anti-apoptotic marker expression, by at least 1%, or at least 2%, or at least 5%, or at least 10%, or at least 20%, or at least 50%, or at least 80%. In particularly preferred embodiments, the reduced cellular apoptosis is associated with increased expression of BCL-2, BCL-XL, BCL-W, A1/BFL-1, or MCL-1 by at least 1%, or at least 2%, or at least 5%, or at least 10%, or at least 20%, or at least 50%, or at least 80%.

In some embodiments, the tissue or organ treated with the isolated mitochondria has increased glucose uptake and decreased lactate production in comparison to a corresponding tissue or organ not treated with the isolated mitochondria. In preferred embodiments, the increased glucose uptake and decreased lactate production is associated with increased expression of HK, VDAC1, GLUT, AKT1, or any combination thereof, by at least 1%, or at least 2%, or at least 5%, or at least 10%, or at least 20%, or at least 50%, or at least 80%.

In some embodiments, the tissue or organ is generated by bioprinting. See, e.g., Murphy, S. V. and Atala, A., Nat Biotechnol. 2004, 32(8):773-85.

Non-limiting examples of improved mitochondrial function are increased oxygen consumption and/or increased adenosine triphosphate (ATP) synthesis, by at least 1%, or at least 2%, or at least 5%, or at least 10%, or at least 20%, or at least 50%, or at least 100%.

Non-limiting examples of routes of delivery of isolated mitochondria to organs or tissues are delivery through the airway of the lung, intravenous delivery and intra-arterial delivery.

### III. Methods of Improving Organ, Tissue, or Lung Function

Disclosed herein is a method of improving the function of a lung subjected to ex vivo lung perfusion (EVLP), the method comprising: (i) delivering isolated mitochondria to a lung, and (ii) performing EVLP on the lung in a chamber or vessel by perfusing the lung with a perfusate solution from a reservoir. In some embodiments, the isolated mitochondria are isolated porcine mitochondria. In some embodiments, the isolated mitochondria are isolated human mitochondria allogeneic to the lung. In some embodiments, the isolated mitochondria are isolated human mitochondria autologous to the lung. In preferred embodiments, cells of the lung treated with the isolated mitochondria have at least 1%, or at least 2%, or at least 5%, or at least 10%, or at least 20%, or at least 50%, or at least 100% improvement in mitochondrial function in comparison to cells of a corresponding lung not treated with the isolated mitochondria. In some embodiments, the lung treated with the isolated mitochondria has enhanced stability or maintenance of one or more EVLP parameters in comparison to a corresponding lung not treated with the isolated mitochondria. In preferred embodiments, the lung treated with the isolated mitochondria has at least 1%, or at least 2%, or at least 5%, or at least 10%, or at least 20%, or at least 50%, or at least 100% improvement in one or more EVLP parameters in comparison to a corresponding lung not treated with the isolated mitochondria. In preferred embodiments, the lung is a human lung.

In preferred embodiments, the lung treated with the isolated mitochondria has improved expression of gap junction markers, reduced reactive oxygen species (ROS)-induced DNA oxidation, reduced production of ROS-mediated oxidative byproducts, reduced ROS-mediated chemokine secretion, reduced levels of inflammatory cytokines, reduced apoptosis, or any combination thereof in comparison to a corresponding lung not treated with the isolated mitochondria. In some embodiments, the gap junction markers comprise junctional adhesion molecule 1 (JAM1) and CD31. In some embodiments, the inflammatory cytokines comprise IL-6, IL-8, and interferon-gamma (IFN-γ). In some embodiments, the ROS-mediated oxidative byproducts comprise 4-hydroxynonenal (4-HNE) and 8-hydroxydeoxyguanosine (8-OHdG). In some embodiments, the ROS-mediated chemokines comprise IL-8, CXCL9, MCP-1, and GROα.

In some embodiments, the method further comprises the step of harvesting the lung from a donor prior to performing EVLP. In other embodiments, the method further comprises the steps of harvesting the lung from a donor prior to performing EVLP and transplanting the lung into a recipient after performing EVLP.

In some embodiments, the recipient is a human recipient suffering from lung disease or disorder. In some embodiments, the lung disease or disorder is pulmonary hypertension, bronchopulmonary dysplasia (BPD), lung fibrosis, asthma, sleep-disordered breathing, or chronic obstructive pulmonary disease (COPD). Non-limiting examples of pulmonary hypertension include pulmonary hypertension due to COPD, chronic thromboembolic pulmonary hypertension (CTEPH), pulmonary arterial hypertension (PAH), pulmonary veno-occlusive disease (PVOD), pulmonary capillary hemangiomatosis (PCH), persistent pulmonary hypertension of the newborn, BPD-induced pulmonary hypertension, pulmonary hypertension secondary to left heart disease, pulmonary hypertension due to lung disease, chronic hypoxia, chronic arterial obstruction, or pulmonary hypertension with unclear or multifactorial mechanisms.

In some embodiments, the isolated mitochondria are delivered to the lung prior to performing EVLP. In other embodiments, the isolated mitochondria are delivered to the lung while performing EVLP. In some embodiments, the isolated mitochondria are delivered to the lung after performing EVLP. In some embodiments, the isolated mitochondria are delivered to the lung prior to the step of harvesting the lung from the donor. In other embodiments, the isolated mitochondria are delivered to the lung after the step of harvesting the lung from the donor. In some embodiments, the isolated mitochondria are delivered to the lung through the airway, intravenously, or intra-arterially prior to the step of harvesting the lung from the donor. In other embodiments, the isolated mitochondria are delivered to the lung through the airway, intravenously, or intra-arterially after the step of harvesting the lung from the donor.

In preferred embodiments, the perfusate solution is introduced into the lung through a cannulated pulmonary artery. In preferred embodiments, the lung is ventilated in the chamber or vessel through a cannulated trachea.

In preferred embodiments, the lung treated with the isolated mitochondria has improved expression of gap junction markers, reduced ROS-induced DNA oxidation, reduced production of ROS-mediated oxidative byproducts, reduced ROS-mediated chemokine secretion, reduced levels of inflammatory cytokines, reduced apoptosis, or any combination thereof in comparison to a corresponding lung not treated with the isolated mitochondria. In some embodiments, the gap junction markers comprise JAM1 and CD31. In some embodiments, the inflammatory cytokines comprise IL-6, IL-8, and IFN-γ. In some embodiments, the ROS-mediated oxidative byproducts comprise 4-HNE and 8-OHdG. In some embodiments, the ROS-mediated chemokines comprise IL-8, CXCL9, MCP-1, and GROα.

In some embodiments, the lung treated with the isolated mitochondria has reduced inflammation and/or immune cell activation in comparison to a corresponding lung not treated with the isolated mitochondria. In preferred embodiments, the reduced inflammation and/or immune cell activation is associated with reduced expression of MAPK14, JNK, or p53, by at least 1%, or at least 2%, or at least 5%, or at least 10%, or at least 20%, or at least 50%, or at least 80%. In preferred embodiments, the reduced inflammation and/or immune cell activation is associated with reduced expression of NF-κB, by at least 1%, or at least 2%, or at least 5%, or at least 10%, or at least 20%, or at least 50%, or at least 80%. In preferred embodiments, the reduced inflammation and/or immune cell activation is associated with reduced secretion of pro-inflammatory cytokines and chemokines such as MIP-1β (CCL4), PDGF-BB, RANTES (CCL5), soluble ICAM-1 (sICAM-1), M-CSF (CSF-1), IL-1β, IL-6, IL-8 (CXCL8), GDF-15, TGF-β1, and any combination thereof, by at least 1%, or at least 2%, or at least 5%, or at least 10%, or at least 20%, or at least 50%, or at least 80%. In preferred embodiments, the reduced inflammation and/or immune cell activation is associated with reduced expression of activation markers such as CD69, CD95, CD30, CD137, CD25 (IL2RA), CD38, CD154 (CD40L), and any combination thereof, by at least 1%, or at least 2%, or at least 5%, or at least 10%, or at least 20%, or at least 50%, or at least 80%. In preferred embodiments, the reduced inflammation and/or immune cell activation is associated with reduced expression or secretion of IL-2, IL-4, IL-5, IL-6, IL-9, IL-13, IL17, TNF-α, IFN-γ, or any combination thereof, by at least 1%, or at least 2%, or at least 5%, or at least 10%, or at least 20%, or at least 50%, or at least 80%.

In some embodiments, the lung treated with the isolated mitochondria has reduced cellular apoptosis, increased cell viability, reduced mitochondrial stress signaling, and/or reduced cell damage in comparison to a corresponding lung not treated with the isolated mitochondria. In preferred embodiments, the reduced cell damage is associated with reduced TLR9 expression, altered HO-1 expression, reduced cytosolic mtDNA, or any combination thereof, by at least 1%, or at least 2%, or at least 5%, or at least 10%, or at least 20%, or at least 50%, or at least 80%. In some embodiments, the altered HO-1 expression is increased HO-1 expression after cold exposure. In preferred embodiments, the reduced cellular apoptosis, increased cell viability, reduced mitochondrial stress signaling, and/or reduced cell damage is associated with reduced NF-κB, MAPK14, JNK, p53 expression, or any combination thereof, by at least 1%, or at least 2%, or at least 5%, or at least 10%, or at least 20%, or at least 50%, or at least 80%. In preferred embodiments, the reduced cellular apoptosis is associated with reduced pro-apoptotic marker expression, by at least 1%, or at least 2%, or at least 5%, or at least 10%, or at least 20%, or at least 50%, or at least 80%. In particularly preferred embodiments, the reduced cellular apoptosis is associated with reduced expression of pro-apoptotic initiators (BIM, PUMA), pro-apoptotic effectors (BAX, BAK), apoptogenic factors (SMAC, DIABLO, BID, BAD, etc.), or any combination thereof, by at least 1%, or at least 2%, or at least 5%, or at least 10%, or at least 20%, or at least 50%, or at least 80%. In preferred embodiments, the reduced cellular apoptosis is associated with increased anti-apoptotic marker expression, by at least 1%, or at least 2%, or at least 5%, or at least 10%, or at least 20%, or at least 50%, or at least 80%. In particularly preferred embodiments, the reduced cellular apoptosis is associated with increased expression of BCL-2, BCL-XL, BCL-W, A1/BF-L1, or MCL-1 by at least 1%, or at least 2%, or at least 5%, or at least 10%, or at least 20%, or at least 50%, or at least 80%.

In some embodiments, the lung treated with the isolated mitochondria has increased glucose uptake and decreased lactate production in comparison to a corresponding lung not treated with the isolated mitochondria. In preferred embodiments, the increased glucose uptake and decreased in lactate production is associated with increased expression of HK, VDAC1, GLUT, AKT1, or any combination thereof, by at least 1%, or at least 2%, or at least 5%, or at least 10%, or at least 20%, or at least 50%, or at least 80%.

Non-limiting examples of stable, maintained, or improved EVLP parameters are: stable or improved pulmonary artery pressure (PAP); improved or maintained tidal volume (TV); improved or maintained dynamic compliance (TV/(peak inspiratory pressure (PIP)—positive end expiratory pressure (PEEP))); increased glucose/lactose ratio; decreased histological measures of cell death (e.g., decreased cell death as measured by TUNEL assay); increased angiogenesis and gap junction formation; stable or improved (i.e., decreased) pulmonary vascular resistance (PVR); reduced lactate production; reduced ammonium production; improved minute ventilation; improved blood flow; reduced pulmonary edema; improved lung elastance; and stable or improved gas exchange. Increased CD31 expression is indicative of angiogenesis and gap junction formation.

Non-limiting examples of perfusate solutions are Steen solution, Perfadex, low-potassium dextran solution, whole blood, diluted blood, packed red blood cells (RBCs), a plasma substitute, one or more vasodilators, sodium bicarbonate, glucose, and any combination thereof.

Non-limiting examples of delivery of isolated mitochondria to lungs are delivery through the airway, delivery from the reservoir of the chamber or vessel, intravenous delivery, and intra-arterial delivery.

Also disclosed herein is a method for minimizing damage to an organ ex vivo due to cold ischemia during transportation, shipment, or storage, the method comprising: delivering isolated mitochondria to the organ 0-24 hours before cold ischemia, during cold ischemia, or 0-24 hours after cold ischemia, wherein cells of the organ treated with the isolated mitochondria have at least 1%, or at least 2%, or at least 5%, or at least 10%, or at least 20%, or at least 50%, or at least 100% improvement in mitochondrial function in comparison to cells of a corresponding organ not treated with the isolated mitochondria, and wherein the improved mitochondrial function is increased oxygen consumption and/or increased ATP synthesis, by at least 1%, or at least 2%, or at least 5%, or at least 10%, or at least 20%, or at least 50%, or at least 100%. In some embodiments, the isolated mitochondria are isolated porcine mitochondria. In some embodiments, the isolated mitochondria are isolated human mitochondria allogeneic to the organ. In some embodiments, the isolated mitochondria are isolated human mitochondria autologous to the organ. In some embodiments, the method further comprises the step of harvesting the organ from a donor. In some embodiments, the isolated mitochondria are delivered to the organ at 0, 1, 2, 3, 4, 5, 6, 7, 8, 9, 10, 11, 12, 13, 14, 15, 16, 17, 18, 19, 20, 21, 22, 23, or 24 hours before cold ischemia. In other embodiments, the isolated mitochondria are delivered to the organ during cold ischemia. In other embodiments, the isolated mitochondria are delivered to the organ at 0, 1, 2, 3, 4, 5, 6, 7, 8, 9, 10, 11, 12, 13, 14, 15, 16, 17, 18, 19, 20, 21, 22, 23, or 24 hours after cold ischemia. In preferred embodiments, the organ is a human organ. In other embodiments, the organ is a pig organ for xenotransplantation into a human subject.

In preferred embodiments, the organ treated with the isolated mitochondria has reduced production of ROS-mediated oxidative byproducts, improved cell viability, reduced necrosis, reduced cell lysis, increased total levels of cellular ATP, reduced inflammatory cytokine secretion, or any combination thereof in comparison to a corresponding organ not treated with the isolated mitochondria. In some embodiments, the inflammatory cytokines comprise IL-6, IL-8, and IFN-γ. In some embodiments, the ROS-mediated oxidative byproducts comprise 4-HNE and 8-OHdG.

In preferred embodiments, the organ treated with the isolated mitochondria is a kidney. In other preferred embodiments, the organ is a kidney, and the method further comprises the step of transplanting the kidney treated with the isolated mitochondria into a human recipient suffering from a kidney disease or disorder. In other preferred embodiments, the organ is a kidney, and the method further comprises the step of harvesting the kidney from a donor. In other preferred embodiments, the organ is a kidney, and the method further comprises the steps of harvesting a kidney from a donor and transplanting the kidney treated with isolated mitochondria into a human recipient suffering from a kidney disease or disorder.

In preferred embodiments, the organ is a lung, and the method further comprises the step of performing EVLP on the lung in a chamber or vessel by perfusing the lung with a perfusate solution from a reservoir. In other preferred embodiments, the organ is a lung, and the method comprises the steps of harvesting the lung from a donor and performing EVLP on the lung in a chamber or vessel by perfusing the lung with a perfusate solution from a reservoir. In other preferred embodiments, the organ is a lung, and the method comprises the steps of harvesting the lung from a donor, performing EVLP on the lung in a chamber or vessel by perfusing the lung with a perfusate solution from a reservoir, and transplanting the lung into a human recipient suffering from pulmonary hypertension. In preferred embodiments, the lung treated with the isolated mitochondria has enhanced stability or maintenance of one or more EVLP parameters in comparison to a corresponding lung not treated with the isolated mitochondria. In particularly preferred embodiments, the lung treated with the isolated mitochondria has at least 1%, or at least 2%, or at least 5%, or at least 10%, or at least 20%, or at least 50%, or at least 100% improvement in one or more EVLP parameters in comparison to a corresponding lung not treated with the isolated mitochondria. In particularly preferred embodiments, the lung is a human lung.

In some embodiments, the isolated mitochondria are delivered to the lung prior to performing EVLP. In other embodiments, the isolated mitochondria are delivered to the lung while performing EVLP. In other embodiments, the isolated mitochondria are delivered to the lung after performing EVLP. In some embodiments, the isolated mitochondria are delivered to the lung prior to the step of harvesting the lung from the donor. In other embodiments, the isolated mitochondria are delivered to the lung after the step of harvesting the lung from the donor.

In preferred embodiments, the perfusate solution is introduced into the lung through a cannulated pulmonary artery. In preferred embodiments, the lung is ventilated in the chamber or vessel through a cannulated trachea.

In some embodiments, the organ, kidney, or lung treated with the isolated mitochondria has reduced inflammation and/or immune cell activation in comparison to a corresponding organ, kidney, or lung not treated with the isolated mitochondria. In preferred embodiments, the reduced inflammation and/or immune cell activation is associated with reduced expression of MAPK14, JNK, or p53, by at least 1%, or at least 2%, or at least 5%, or at least 10%, or at least 20%, or at least 50%, or at least 80%. In preferred embodiments, the reduced inflammation and/or immune cell activation is associated with reduced expression of NF-κB, by at least 1%, or at least 2%, or at least 5%, or at least 10%, or at least 20%, or at least 50%, or at least 80%. In preferred embodiments, the reduced inflammation and/or immune cell activation is associated with reduced secretion of pro-inflammatory cytokines and chemokines such as MIP-1β (CCL4), PDGF-BB, RANTES (CCL5), soluble ICAM-1 (sICAM-1), M-CSF (CSF-1), IL-1β, IL-6, IL-8 (CXCL8), GDF-15, TGF-β1, and any combination thereof, by at least 1%, or at least 2%, or at least 5%, or at least 10%, or at least 20%, or at least 50%, or at least 80%. In preferred embodiments, the reduced inflammation and/or immune cell activation is associated with reduced expression of activation markers such as CD69, CD95, CD30, CD137, CD25 (IL2RA), CD38, CD154 (CD40L), and any combination thereof, by at least 1%, or at least 2%, or at least 5%, or at least 10%, or at least 20%, or at least 50%, or at least 80%. In preferred embodiments, the reduced inflammation and/or immune cell activation is associated with reduced expression or secretion of IL-2, IL-4, IL-5, IL-6, IL-9, IL-13, IL17, TNF-α, IFN-γ, or any combination thereof, by at least 1%, or at least 2%, or at least 5%, or at least 10%, or at least 20%, or at least 50%, or at least 80%.

In some embodiments, the organ, kidney, or lung treated with the isolated mitochondria has reduced cellular apoptosis, increased cell viability, reduced mitochondrial stress signaling, and/or reduced cell damage in comparison to a corresponding organ, kidney, or lung not treated with the isolated mitochondria. In preferred embodiments, the reduced cell damage is associated with reduced TLR9 expression, altered HO-1 expression, reduced cytosolic mtDNA, or any combination thereof, by at least 1%, or at least 2%, or at least 5%, or at least 10%, or at least 20%, or at least 50%, or at least 80%. In some embodiments, the altered HO-1 expression is increased HO-1 expression after cold exposure. In preferred embodiments, the reduced cellular apoptosis, increased cell viability, reduced mitochondrial stress signaling, and/or reduced cell damage is associated with reduced NF-κB, MAPK14, JNK, p53 expression, or any combination thereof, by at least 1%, or at least 2%, or at least 5%, or at least 10%, or at least 20%, or at least 50%, or at least 80%. In preferred embodiments, the reduced cellular apoptosis is associated with reduced pro-apoptotic marker expression, by at least 1%, or at least 2%, or at least 5%, or at least 10%, or at least 20%, or at least 50%, or at least 80%. In particularly preferred embodiments, the reduced cellular apoptosis is associated with reduced expression of pro-apoptotic initiators (BIM, PUMA), pro-apoptotic effectors (BAX, BAK), apoptogenic factors (SMAC, DIABLO, BID, BAD, etc.), or any combination thereof, by at least 1%, or at least 2%, or at least 5%, or at least 10%, or at least 20%, or at least 50%, or at least 80%. In particularly preferred embodiments, the reduced cellular apoptosis is associated with increased anti-apoptotic marker expression, by at least 1%, or at least 2%, or at least 5%, or at least 10%, or at least 20%, or at least 50%, or at least 80%. In particularly preferred embodiments, the reduced cellular apoptosis is associated with increased expression of BCL-2, BCL-XL, BCL-W, A1/BFL-1, or MCL-1 by at least 1%, or at least 2%, or at least 5%, or at least 10%, or at least 20%, or at least 50%, or at least 80%.

In some embodiments, the organ, kidney, or lung treated with the isolated mitochondria has increased glucose uptake and decreased lactate production in comparison to a corresponding organ, kidney, or lung not treated with the isolated mitochondria. In preferred embodiments, the increased glucose uptake and decreased lactate production is associated with increased expression of HK, VDAC1, GLUT, AKT1, or any combination thereof, by at least 1%, or at least 2%, or at least 5%, or at least 10%, or at least 20%, or at least 50%, or at least 80%.

Also disclosed herein is a method for improving the function of an engineered organ or tissue, the method comprising: (i) preparing an organ or tissue scaffold comprising one or more extracellular matrix components, (ii) populating the organ or tissue scaffold in a bioreactor, chamber, or vessel with populating cells to produce an engineered organ or tissue, and (iii) delivering isolated mitochondria to the engineered organ or tissue. In some embodiments, the isolated mitochondria are isolated porcine mitochondria. In some embodiments, the isolated mitochondria are isolated human mitochondria allogeneic to the engineered organ or tissue. In some embodiments, the isolated mitochondria are isolated human mitochondria autologous to the engineered organ or tissue. In preferred embodiments, cells of the engineered organ or tissue treated with the isolated mitochondria have at least 1%, or at least 2%, or at least 5%, or at least 10%, or at least 20%, or at least 50%, or at least 100% improvement in mitochondrial function in comparison to cells of a corresponding engineered organ not treated with the isolated mitochondria. In particularly preferred embodiments, the engineered organ or tissue treated with the isolated mitochondria has one or more improved cellular, organ, or tissue functions in comparison to a corresponding engineered organ or tissue not treated with the isolated mitochondria, wherein the one or more improved cellular, organ or tissue functions are increased cell adherence to the scaffold, increased cell viability, reduced apoptosis, reduced cell damage, increased cell proliferation, increased cellular barrier function, reduced DNA damage, increased angiogenesis, improved blood vessel maintenance, reduced mitochondrial stress signaling, reduced reactive oxygen species production, or any combination thereof. In preferred embodiments, the engineered organ or tissue treated with the isolated mitochondria is an engineered human organ or tissue.

In some embodiments, the engineered organ or tissue treated with the isolated mitochondria is an engineered human kidney. In some embodiments, the engineered human organ or tissue treated with the isolated mitochondria is an engineered human lung. In preferred embodiments, the engineered human lung treated with the isolated mitochondria has enhanced stability or maintenance of one or more EVLP parameters in comparison to a corresponding engineered lung not treated with the isolated mitochondria. In particularly preferred embodiments, the engineered human lung treated with the isolated mitochondria has enhanced stability or maintenance of PAP; TV; dynamic compliance; PVR; gas exchange; or any combination thereof in comparison to a corresponding engineered human lung not treated with the isolated mitochondria. In preferred embodiments, the engineered human lung treated with the isolated mitochondria has at least 1%, or at least 2%, or at least 5%, or at least 10%, or at least 20%, or at least 50%, or at least 100% improvement in one or more EVLP parameters in comparison to a corresponding lung not treated with the isolated mitochondria. In particularly preferred embodiments, the improvement in one or more EVLP parameters is improved PAP; improved TV; improved dynamic compliance; increased glucose/lactose ratio; decreased histological measures of cell death; increased angiogenesis and gap junction formation; decreased PVR; reduced lactate production; reduced ammonium production; improved minute ventilation; improved blood flow; reduced pulmonary edema; improved lung elastance; improved gas exchange; or any combination thereof.

In preferred embodiments, the engineered human lung treated with the isolated mitochondria has improved expression of gap junction markers, reduced ROS-induced DNA oxidation, reduced production of ROS-mediated oxidative byproducts, reduced ROS-mediated chemokine secretion, reduced levels of inflammatory cytokines, reduced apoptosis, or any combination thereof in comparison to a corresponding engineered human lung not treated with the isolated mitochondria. In some embodiments, the gap junction markers comprise JAM1 and CD31. In some embodiments, the inflammatory cytokines comprise IL-6, IL-8, and IFN-γ. In some embodiments, the ROS-mediated oxidative byproducts comprise 4-HNE and 8-OHdG. In some embodiments, the ROS-mediated chemokines comprise IL-8, CXCL9, MCP-1, and GROα.

In some embodiments, the isolated mitochondria are delivered to the engineered organ or tissue after the step of populating the organ or tissue scaffold. In other embodiments, the isolated mitochondria are delivered to the engineered organ or tissue during the step of populating the organ or tissue scaffold. In preferred embodiments, the isolated mitochondria are delivered to the engineered organ or tissue together with the populating cells in the bioreactor, chamber, or vessel during the step of populating the organ or tissue scaffold.

In some embodiments, the organ or tissue scaffold is infused with isolated mitochondria prior to populating the organ or tissue scaffold in the bioreactor, chamber, or vessel.

In some embodiments, the organ or tissue scaffold is generated by bioprinting. In preferred embodiments, the populating cells and the artificial organ or tissue matrix are bioprinted concurrently to produce the engineered organ or tissue. See, e.g., Murphy, S. V. and Atala, A., Nat Biotechnol. 2004, 32(8):773-85.

In some embodiments, the engineered organ or tissue treated with the isolated mitochondria has reduced inflammation and/or immune cell activation in comparison to a corresponding engineered organ or tissue not treated with the isolated mitochondria. In preferred embodiments, the reduced inflammation and/or immune cell activation is associated with reduced expression of MAPK14, JNK, or p53, by at least 1%, or at least 2%, or at least 5%, or at least 10%, or at least 20%, or at least 50%, or at least 80%. In preferred embodiments, the reduced inflammation and/or immune cell activation is associated with reduced expression of NF-κB, by at least 1%, or at least 2%, or at least 5%, or at least 10%, or at least 20%, or at least 50%, or at least 80%. In preferred embodiments, the reduced inflammation and/or immune cell activation is associated with reduced secretion of MIP-1β (CCL4), PDGF-BB, RANTES (CCL5), soluble ICAM-1 (sICAM-1), M-CSF (CSF-1), IL-1β, IL-6, IL-8 (CXCL8), GDF-15, TGF-β1, and any combination thereof, by at least 1%, or at least 2%, or at least 5%, or at least 10%, or at least 20%, or at least 50%, or at least 80%. In preferred embodiments, the reduced inflammation and/or immune cell activation is associated with reduced expression of activation markers such as CD69, CD95, CD30, CD137, CD25 (IL2RA), CD38, CD154 (CD40L), and any combination thereof, by at least 1%, or at least 2%, or at least 5%, or at least 10%, or at least 20%, or at least 50%, or at least 80%. In preferred embodiments, the reduced inflammation and/or immune cell activation is associated with reduced expression or secretion of IL-2, IL-4, IL-5, IL-6, IL-9, IL-13, IL17, TNF-α, IFN-γ, or any combination thereof, by at least 1%, or at least 2%, or at least 5%, or at least 10%, or at least 20%, or at least 50%, or at least 80%.

In some embodiments, the engineered organ or tissue treated with the isolated mitochondria has reduced cellular apoptosis, increased cell viability, reduced mitochondrial stress signaling, and/or reduced cell damage in comparison to a corresponding engineered organ or tissue not treated with the isolated mitochondria. In preferred embodiments, the reduced cell damage is associated with reduced TLR9 expression, altered HO-1 expression, reduced cytosolic mtDNA, or any combination thereof, by at least 1%, or at least 2%, or at least 5%, or at least 10%, or at least 20%, or at least 50%, or at least 80%. In some embodiments, the altered HO-1 expression is increased HO-1 expression after cold exposure. In preferred embodiments, the reduced cellular apoptosis, increased cell viability, reduced mitochondrial stress signaling, and/or reduced cell damage is associated with reduced NF-κB, MAPK14, JNK, p53 expression, or any combination thereof, by at least 1%, or at least 2%, or at least 5%, or at least 10%, or at least 20%, or at least 50%, or at least 80%. In preferred embodiments, the reduced cellular apoptosis is associated with reduced pro-apoptotic marker expression, by at least 1%, or at least 2%, or at least 5%, or at least 10%, or at least 20%, or at least 50%, or at least 80%. In particularly preferred embodiments, the reduced cellular apoptosis is associated with reduced expression of pro-apoptotic initiators (BIM, PUMA), pro-apoptotic effectors (BAX, BAK), apoptogenic factors (SMAC, DIABLO, BID, BAD, etc.), or any combination thereof, by at least 1%, or at least 2%, or at least 5%, or at least 10%, or at least 20%, or at least 50%, or at least 80%. In preferred embodiments, the reduced cellular apoptosis is associated with increased anti-apoptotic marker expression, by at least 1%, or at least 2%, or at least 5%, or at least 10%, or at least 20%, or at least 50%, or at least 80%. In particularly preferred embodiments, the reduced cellular apoptosis is associated with increased expression of BCL-2, BCL-XL, BCL-W, A1/BFL-1, or MCL-1 by at least 1%, or at least 2%, or at least 5%, or at least 10%, or at least 20%, or at least 50%, or at least 80%.

In some embodiments, the engineered organ or tissue treated with the isolated mitochondria has increased glucose uptake and decreased lactate production in comparison to a corresponding engineered organ or tissue not treated with the isolated mitochondria. In preferred embodiments, the increased glucose uptake and decreased lactate production is associated with increased expression of HK, VDAC1, GLUT, AKT1, or any combination thereof, by at least 1%, or at least 2%, or at least 5%, or at least 10%, or at least 20%, or at least 50%, or at least 80%.

Non-limiting examples of populating cells are epithelial cells (e.g., type I alveolar cells, type II alveolar cells, small and large airway epithelial cells), endothelial cells (e.g., human pulmonary artery endothelial cells (HPAEC)), fibroblasts, progenitor cells (e.g., endothelial progenitor cells and mesenchymal stem cells), smooth muscle cells (e.g., pulmonary artery smooth muscle cells), immune cells, mesenchymal cells, pericytes, and any combination thereof.

Non-limiting examples of delivery of the isolated mitochondria to engineered organs and tissues are intravenous delivery, intra-arterial delivery, intra-tracheal delivery, or delivery by perfusion, or delivery via the lymphatic system or the bronchial circulation.

Also disclosed herein is a method for improving the function of an engineered organ or tissue, the method comprising: (i) preparing an organ or tissue scaffold comprising one or more extracellular matrix components, and (ii) populating the organ or tissue scaffold in a bioreactor, chamber, or vessel with the populating cells treated with isolated mitochondria to produce an engineered organ or tissue. In some embodiments, the isolated mitochondria are isolated porcine mitochondria. In some embodiments, the isolated mitochondria are isolated human mitochondria allogeneic to the engineered organ or tissue. In some embodiments, the isolated mitochondria are isolated human mitochondria autologous to the engineered organ or tissue. In preferred embodiments, cells of the engineered organ or tissue treated with the isolated mitochondria have at least 1%, or at least 2%, or at least 5%, or at least 10%, or at least 20%, or at least 50%, or at least 100% improvement in mitochondrial function in comparison to cells of a corresponding engineered organ not treated with the isolated mitochondria. In particularly preferred embodiments, the engineered organ or tissue treated with the isolated mitochondria has one or more improved cellular, organ, or tissue functions in comparison to a corresponding engineered organ or tissue not treated with the isolated mitochondria, wherein the one or more improved cellular, organ, or tissue functions are increased cell adherence to the scaffold, increased cell viability, reduced apoptosis, reduced cell damage, increased cell proliferation, increased cellular barrier function, reduced DNA damage, increased angiogenesis, improved blood vessel maintenance, reduced mitochondrial stress signaling, reduced reactive oxygen species production, or any combination thereof. In preferred embodiments, the engineered organ or tissue treated with the isolated mitochondria is an engineered human organ or tissue.

In some embodiments, the engineered human organ or tissue treated with the isolated mitochondria is an engineered human lung. In preferred embodiments, the engineered human lung treated with the isolated mitochondria has enhanced stability or maintenance of one or more EVLP parameters in comparison to a corresponding engineered lung not treated with the isolated mitochondria. In particularly preferred embodiments, the engineered human lung treated with the isolated mitochondria has enhanced stability or maintenance of PAP; TV; dynamic compliance; PVR; gas exchange; or any combination thereof in comparison to a corresponding engineered human lung not treated with the isolated mitochondria. In preferred embodiments, the engineered human lung treated with the isolated mitochondria has at least 1%, or at least 2%, or at least 5%, or at least 10%, or at least 20%, or at least 50%, or at least 100% improvement in one or more EVLP parameters in comparison to a corresponding lung not treated with the isolated mitochondria. In particularly preferred embodiments, the improvement in one or more EVLP parameters is improved PAP; improved TV; improved dynamic compliance; increased glucose/lactose ratio; decreased histological measures of cell death; increased angiogenesis and gap junction formation; decreased PVR; reduced lactate production; reduced ammonium production; improved minute ventilation; improved blood flow; reduced pulmonary edema; improved lung elastance; improved gas exchange; or any combination thereof.

In preferred embodiments, the engineered human lung treated with the isolated mitochondria has improved expression of gap junction markers, reduced ROS-induced DNA oxidation, reduced production of ROS-mediated oxidative byproducts, reduced ROS-mediated chemokine secretion, reduced levels of inflammatory cytokines, reduced apoptosis, or any combination thereof in comparison to a corresponding engineered human lung not treated with the isolated mitochondria. In some embodiments, the gap junction markers comprise JAM1 and CD31. In some embodiments, the inflammatory cytokines comprise IL-6, IL-8, and IFN-γ. In some embodiments, the ROS-mediated oxidative byproducts comprise 4-HNE and 8-OHdG. In some embodiments, the ROS-mediated chemokines comprise IL-8, CXCL9, MCP-1, and GROα.

In some embodiments, the organ or tissue scaffold is infused with isolated mitochondria prior to populating the organ or tissue scaffold in the bioreactor, chamber, or vessel.

In some embodiments, the organ or tissue scaffold is generated by bioprinting. In preferred embodiments, the populating cells and the artificial organ or tissue matrix are bioprinted concurrently to produce the engineered organ or tissue. See, e.g., Murphy, S. V. and Atala, A., Nat Biotechnol. 2004, 32(8):773-85.

In some embodiments, the engineered organ or tissue treated with the isolated mitochondria has reduced inflammation and/or immune cell activation in comparison to a corresponding engineered organ or tissue not treated with the isolated mitochondria. In preferred embodiments, the reduced inflammation and/or immune cell activation is associated with reduced expression of MAPK14, JNK, or p53, by at least 1%, or at least 2%, or at least 5%, or at least 10%, or at least 20%, or at least 50%, or at least 80%. In preferred embodiments, the reduced inflammation and/or immune cell activation is associated with reduced expression of NF-κB, by at least 1%, or at least 2%, or at least 5%, or at least 10%, or at least 20%, or at least 50%, or at least 80%. In preferred embodiments, the reduced inflammation and/or immune cell activation is associated with reduced secretion of pro-inflammatory cytokines and chemokines such as MIP-1β (CCL4), PDGF-BB, RANTES (CCL5), soluble ICAM-1 (sICAM-1), M-CSF (CSF-1), IL-1β, IL-6, IL-8 (CXCL8), GDF-15, TGF-β1, and any combination thereof, by at least 1%, or at least 2%, or at least 5%, or at least 10%, or at least 20%, or at least 50%, or at least 80%. In preferred embodiments, the reduced inflammation and/or immune cell activation is associated with reduced expression of activation markers such as CD69, CD95, CD30, CD137, CD25 (IL2RA), CD38, CD154 (CD40L), and any combination thereof, by at least 1%, or at least 2%, or at least 5%, or at least 10%, or at least 20%, or at least 50%, or at least 80%. In preferred embodiments, the reduced inflammation and/or immune cell activation is associated with reduced expression or secretion of IL-2, IL-4, IL-5, IL-6, IL-9, IL-13, IL17, TNF-α, IFN-γ, or any combination thereof, by at least 1%, or at least 2%, or at least 5%, or at least 10%, or at least 20%, or at least 50%, or at least 80%.

In some embodiments, the engineered organ or tissue treated with the isolated mitochondria has reduced cellular apoptosis, increased cell viability, reduced mitochondrial stress signaling, and/or reduced cell damage in comparison to a corresponding engineered organ or tissue not treated with the isolated mitochondria. In preferred embodiments, the reduced cell damage is associated with reduced TLR9 expression, altered HO-1 expression, reduced cytosolic mtDNA, or any combination thereof, by at least 1%, or at least 2%, or at least 5%, or at least 10%, or at least 20%, or at least 50%, or at least 80%. In some embodiments, the altered HO-1 expression is increased HO-1 expression after cold exposure. In preferred embodiments, the reduced cellular apoptosis, increased cell viability, reduced mitochondrial stress signaling, and/or reduced cell damage is associated with reduced NF-κB, MAPK14, JNK, p53 expression, or any combination thereof, by at least 1%, or at least 2%, or at least 5%, or at least 10%, or at least 20%, or at least 50%, or at least 80%. In preferred embodiments, the reduced cellular apoptosis is associated with reduced pro-apoptotic marker expression, by at least 1%, or at least 2%, or at least 5%, or at least 10%, or at least 20%, or at least 50%, or at least 80%. In particularly preferred embodiments, the reduced cellular apoptosis is associated with reduced expression of pro-apoptotic initiators (BIM, PUMA), pro-apoptotic effectors (BAX, BAK), apoptogenic factors (SMAC, DIABLO, BID, BAD, etc.), or any combination thereof, by at least 1%, or at least 2%, or at least 5%, or at least 10%, or at least 20%, or at least 50%, or at least 80%. In preferred embodiments, the reduced cellular apoptosis is associated with increased anti-apoptotic marker expression, by at least 1%, or at least 2%, or at least 5%, or at least 10%, or at least 20%, or at least 50%, or at least 80%. In particularly preferred embodiments, the reduced cellular apoptosis is associated with increased expression of BCL-2, BCL-XL, BCL-W, A1/BFL-1, or MCL-1 by at least 1%, or at least 2%, or at least 5%, or at least 10%, or at least 20%, or at least 50%, or at least 80%.

In some embodiments, the engineered organ or tissue treated with the isolated mitochondria has increased glucose uptake and decreased lactate production in comparison to a corresponding engineered organ or tissue not treated with the isolated mitochondria. In preferred embodiments, the increased glucose uptake and decreased lactate production is associated with increased expression of HK, VDAC1, GLUT, AKT1, or any combination thereof, by at least 1%, or at least 2%, or at least 5%, or at least 10%, or at least 20%, or at least 50%, or at least 80%.

Also disclosed herein is a method for improving the function of an engineered organ or tissue, the method comprising: (i) preparing an organ or tissue scaffold comprising one or more extracellular matrix components, (ii) infusing the organ or tissue scaffold with isolated mitochondria, and (iii) populating the infused organ or tissue scaffold in a bioreactor, chamber, or vessel with populating cells to produce an engineered organ or tissue. In some embodiments, the isolated mitochondria are isolated porcine mitochondria. In some embodiments, the isolated mitochondria are isolated human mitochondria allogeneic to the engineered organ or tissue. In some embodiments, the isolated mitochondria are isolated mitochondria autologous to the engineered organ or tissue. In preferred embodiments, cells of the engineered lung have at least 1%, or at least 2%, or at least 5%, or at least 10%, or at least 20%, or at least 50%, or at least 100%, improvement in mitochondrial function in comparison to cells of a corresponding engineered lung not treated with the isolated mitochondria. In particularly preferred embodiments, the engineered organ or tissue has one or more improved cellular, organ, or tissue functions in comparison to a corresponding engineered organ or tissue not treated with the isolated mitochondria, wherein the one or more improved cellular, organ, or tissue functions are increased cell adherence to the scaffold, increased cell viability, reduced apoptosis, reduced cell damage, increased cell proliferation, increased cellular barrier function, reduced DNA damage, increased angiogenesis, improved blood vessel maintenance, reduced mitochondrial stress signaling, reduced reactive oxygen species production, or any combination thereof. In preferred embodiments, the engineered organ or tissue is an engineered human organ or tissue.

In some embodiments, the engineered organ or tissue is an engineered human kidney. In some embodiments, the engineered human organ or tissue is an engineered human lung. In preferred embodiments, the engineered human has enhanced stability or maintenance of one or more EVLP parameters in comparison to a corresponding engineered lung not treated with the isolated mitochondria. In particularly preferred embodiments, the engineered human lung has enhanced stability or maintenance of PAP; TV; dynamic compliance; PVR; gas exchange; or any combination thereof in comparison to a corresponding engineered human lung not treated with the isolated mitochondria. In preferred embodiments, the engineered human lung has at least 1%, or at least 2%, or at least 5%, or at least 10%, or at least 20%, or at least 50%, or at least 100% improvement in one or more EVLP parameters in comparison to a corresponding lung not treated with the isolated mitochondria. In particularly preferred embodiments, the improvement in one or more EVLP parameters is improved PAP; improved TV; improved dynamic compliance; increased glucose/lactose ratio; decreased histological measures of cell death; increased angiogenesis and gap junction formation; decreased PVR; reduced lactate production; reduced ammonium production; improved minute ventilation; improved blood flow; reduced pulmonary edema; improved lung elastance; improved gas exchange; or any combination thereof.

In preferred embodiments, the engineered human lung treated with the isolated mitochondria has improved expression of gap junction markers, reduced ROS-induced DNA oxidation, reduced production of ROS-mediated oxidative byproducts, reduced ROS-mediated chemokine secretion, reduced levels of inflammatory cytokines, reduced apoptosis, or any combination thereof in comparison to a corresponding human engineered lung not treated with the isolated mitochondria. In some embodiments, the gap junction markers comprise JAM1 and CD31. In some embodiments, the inflammatory cytokines comprise IL-6, IL-8, and IFN-γ. In some embodiments, the ROS-mediated oxidative byproducts comprise 4-HNE and 8-OHdG. In some embodiments, the ROS-mediated chemokines comprise IL-8, CXCL9, MCP-1, and GROα.

In some embodiments, the organ or tissue scaffold is generated by bioprinting. In preferred embodiments, the populating cells and the artificial organ or tissue matrix are bioprinted concurrently to produce the engineered organ or tissue. See, e.g., Murphy, S. V. and Atala, A., Nat Biotechnol. 2004, 32(8):773-85.

In some embodiments, the engineered organ or tissue has reduced inflammation and/or immune cell activation in comparison to a corresponding engineered organ or tissue not treated with the isolated mitochondria. In preferred embodiments, the reduced inflammation and/or immune cell activation is associated with reduced expression of MAPK14, JNK, or p53, by at least 1%, or at least 2%, or at least 5%, or at least 10%, or at least 20%, or at least 50%, or at least 80%. In preferred embodiments, the reduced inflammation and/or immune cell activation is associated with reduced expression of NF-κB, by at least 1%, or at least 2%, or at least 5%, or at least 10%, or at least 20%, or at least 50%, or at least 80%. In preferred embodiments, the reduced inflammation and/or immune cell activation is associated with reduced secretion of pro-inflammatory cytokines and chemokines such as MIP-1β (CCL4), PDGF-BB, RANTES (CCL5), soluble ICAM-1 (sICAM-1), M-CSF (CSF-1), IL-1β, IL-6, IL-8 (CXCL8), GDF-15, TGF-β1, and any combination thereof, by at least 1%, or at least 2%, or at least 5%, or at least 10%, or at least 20%, or at least 50%, or at least 80%. In preferred embodiments, the reduced inflammation and/or immune cell activation is associated with reduced expression of activation markers such as CD69, CD95, CD30, CD137, CD25 (IL2RA), CD38, CD154 (CD40L), and any combination thereof, by at least 1%, or at least 2%, or at least 5%, or at least 10%, or at least 20%, or at least 50%, or at least 80%. In preferred embodiments, the reduced inflammation and/or immune cell activation is associated with reduced expression or secretion of IL-2, IL-4, IL-5, IL-6, IL-9, IL-13, IL17, TNF-α, IFN-γ, or any combination thereof, by at least 1%, or at least 2%, or at least 5%, or at least 10%, or at least 20%, or at least 50%, or at least 80%.

In some embodiments, the engineered organ or tissue has reduced cellular apoptosis, increased cell viability, reduced mitochondrial stress signaling, and/or reduced cell damage in comparison to a corresponding engineered organ or tissue not treated with the isolated mitochondria. In preferred embodiments, the reduced cell damage is associated with reduced TLR9 expression, altered HO-1 expression, reduced cytosolic mtDNA, or any combination thereof, by at least 1%, or at least 2%, or at least 5%, or at least 10%, or at least 20%, or at least 50%, or at least 80%. In some embodiments, the altered HO-1 expression is increased HO-1 expression after cold exposure. In preferred embodiments, the reduced cellular apoptosis, increased cell viability, reduced mitochondrial stress signaling, and/or reduced cell damage is associated with reduced NF-κB, MAPK14, JNK, p53 expression, or any combination thereof, by at least 1%, or at least 2%, or at least 5%, or at least 10%, or at least 20%, or at least 50%, or at least 80%. In preferred embodiments, the reduced cellular apoptosis is associated with reduced pro-apoptotic marker expression, by at least 1%, or at least 2%, or at least 5%, or at least 10%, or at least 20%, or at least 50%, or at least 80%. In particularly preferred embodiments, the reduced cellular apoptosis is associated with reduced expression of pro-apoptotic initiators (BIM, PUMA), pro-apoptotic effectors (BAX, BAK), apoptogenic factors (SMAC, DIABLO, BID, BAD, etc.), or any combination thereof, by at least 1%, or at least 2%, or at least 5%, or at least 10%, or at least 20%, or at least 50%, or at least 80%. In preferred embodiments, the reduced cellular apoptosis is associated with increased anti-apoptotic marker expression, by at least 1%, or at least 2%, or at least 5%, or at least 10%, or at least 20%, or at least 50%, or at least 80%. In particularly preferred embodiments, the reduced cellular apoptosis is associated with increased expression of BCL-2, BCL-XL, BCL-W, A1/BFL-1, or MCL-1 by at least 1%, or at least 2%, or at least 5%, or at least 10%, or at least 20%, or at least 50%, or at least 80%.

In some embodiments, the engineered organ or tissue has increased glucose uptake and decreased lactate production in comparison to a corresponding engineered organ or tissue not treated with the isolated mitochondria. In preferred embodiments, the increased glucose uptake and decreased lactate production is associated with increased expression of HK, VDAC1, GLUT, AKT1, or any combination thereof, by at least 1%, or at least 2%, or at least 5%, or at least 10%, or at least 20%, or at least 50%, or at least 80%.

Also disclosed herein is a method for improving the function of an engineered lung, the method comprising: (i) repopulating a decellularized scaffold lung in a bioreactor, chamber, or vessel with repopulating cells to produce an engineered lung, and (ii) delivering isolated mitochondria to the engineered lung. In some embodiments, the isolated mitochondria are isolated porcine mitochondria. In some embodiments, the isolated mitochondria are isolated human mitochondria allogeneic to the engineered lung. In some embodiments, the isolated mitochondria are isolated human mitochondria autologous to the engineered lung. In preferred embodiments, cells of the engineered lung treated with the isolated mitochondria have at least 1%, or at least 2%, or at least 5%, or at least 10%, or at least 20%, or at least 50%, or at least 100%, improvement in mitochondrial function in comparison to cells of a corresponding engineered lung not treated with the isolated mitochondria. In particularly preferred embodiments, the engineered lung treated with the isolated mitochondria has one or more improved cellular, organ, or tissue functions in comparison to a corresponding engineered lung not treated with the isolated mitochondria, wherein the one or more improved cellular, organ, or tissue functions are increased cell adherence to the scaffold, increased cell viability, reduced apoptosis, reduced cell damage, increased cell proliferation, increased cellular barrier function, reduced DNA damage, increased angiogenesis, improved blood vessel maintenance, reduced mitochondrial stress signaling, reduced reactive oxygen species production, or any combination thereof. In preferred embodiments, the engineered lung is an engineered human lung.

In some embodiments, the isolated mitochondria are delivered to the engineered lung after the step of repopulating the decellularized scaffold lung. In other embodiments, the isolated mitochondria are delivered to the engineered lung during the step of repopulating the decellularized scaffold lung. In preferred embodiments, the isolated mitochondria are delivered to the engineered lung together with the repopulating cells in the bioreactor, chamber, or vessel during the step of repopulating the decellularized scaffold lung. In particularly preferred embodiments, the isolated mitochondria are delivered to the engineered lung through the airway, intravenously, or intra-arterially.

In some embodiments, the method further comprises the step of performing EVLP on the engineered lung by perfusing the engineered lung with a perfusate solution from a reservoir. In preferred embodiments, the engineered lung treated with the isolated mitochondria has enhanced stability or maintenance of one or more EVLP parameters in comparison to a corresponding lung not treated with the isolated mitochondria. In particularly preferred embodiments, the engineered human lung treated with the isolated mitochondria has enhanced stability or maintenance of PAP; TV; dynamic compliance; PVR; gas exchange; or any combination thereof in comparison to a corresponding engineered human lung not treated with the isolated mitochondria. In preferred embodiments, the engineered lung treated with the isolated mitochondria has at least 1%, or at least 2%, or at least 5%, or at least 10%, or at least 20%, or at least 50%, or at least 100%, improvement in one or more EVLP parameters in comparison to a corresponding lung not treated with the isolated mitochondria. In particularly preferred embodiments, the improvement in one or more EVLP parameters is improved PAP; improved TV; improved dynamic compliance; increased glucose/lactose ratio; decreased histological measures of cell death; increased angiogenesis and gap junction formation; decreased PVR; reduced lactate production; reduced ammonium production; improved minute ventilation; improved blood flow; reduced pulmonary edema; improved lung elastance; improved gas exchange; or any combination thereof. In some embodiments, the isolated mitochondria are delivered to the engineered lung prior to performing EVLP. In other embodiments, the isolated mitochondria are delivered to the engineered lung while performing EVLP. In some embodiments, the isolated mitochondria are delivered to the engineered lung through the airway, intravenously, or intra-arterially. In other embodiments, the isolated mitochondria are delivered to the engineered lung from the reservoir.

In some embodiments, the perfusate solution is introduced into the engineered lung through a cannulated pulmonary artery. Non-limiting examples of perfusate solutions are Steen solution, Perfadex, low-potassium dextran solution, whole blood, diluted blood, packed RBCs, a plasma substitute, one or more vasodilators, sodium bicarbonate, glucose, and any combination thereof. In some embodiments, the engineered lung is ventilated in the chamber or vessel through a cannulated trachea.

In some embodiments, the engineered lung treated with the isolated mitochondria has reduced inflammation and/or immune cell activation in comparison to a corresponding engineered lung not treated with the isolated mitochondria. In preferred embodiments, the reduced inflammation and/or immune cell activation is associated with reduced expression of MAPK14, JNK, or p53, by at least 1%, or at least 2%, or at least 5%, or at least 10%, or at least 20%, or at least 50%, or at least 80%. In preferred embodiments, the reduced inflammation and/or immune cell activation is associated with reduced expression of NF-κB, by at least 1%, or at least 2%, or at least 5%, or at least 10%, or at least 20%, or at least 50%, or at least 80%. In preferred embodiments, the reduced inflammation and/or immune cell activation is associated with reduced secretion of pro-inflammatory cytokines and chemokines such as MIP-1β (CCL4), PDGF-BB, RANTES (CCL5), soluble ICAM-1 (sICAM-1), M-CSF (CSF-1), IL-1β, IL-6, IL-8 (CXCL8), GDF-15, TGF-β1, and any combination thereof, by at least 1%, or at least 2%, or at least 5%, or at least 10%, or at least 20%, or at least 50%, or at least 80%. In preferred embodiments, the reduced inflammation and/or immune cell activation is associated with reduced expression of activation markers such as CD69, CD95, CD30, CD137, CD25 (IL2RA), CD38, CD154 (CD40L), and any combination thereof, by at least 1%, or at least 2%, or at least 5%, or at least 10%, or at least 20%, or at least 50%, or at least 80%. In preferred embodiments, the reduced inflammation and/or immune cell activation is associated with reduced expression or secretion of IL-2, IL-4, IL-5, IL-6, IL-9, IL-13, IL17, TNF-α, IFN-γ, or any combination thereof, by at least 1%, or at least 2%, or at least 5%, or at least 10%, or at least 20%, or at least 50%, or at least 80%.

In some embodiments, the engineered lung treated with the isolated mitochondria has reduced cellular apoptosis, increased cell viability, reduced mitochondrial stress signaling, and/or reduced cell damage in comparison to a corresponding engineered lung not treated with the isolated mitochondria. In preferred embodiments, the reduced cell damage is associated with reduced TLR9 expression, altered HO-1 expression, reduced cytosolic mtDNA, or any combination thereof, by at least 1%, or at least 2%, or at least 5%, or at least 10%, or at least 20%, or at least 50%, or at least 80%. In some embodiments, the altered HO-1 expression is increased HO-1 expression after cold exposure. In preferred embodiments, the reduced cellular apoptosis, increased cell viability, reduced mitochondrial stress signaling, and/or reduced cell damage is associated with reduced NF-κB, MAPK14, JNK, p53 expression, or any combination thereof, by at least 1%, or at least 2%, or at least 5%, or at least 10%, or at least 20%, or at least 50%, or at least 80%. In preferred embodiments, the reduced cellular apoptosis is associated with reduced pro-apoptotic marker expression, by at least 1%, or at least 2%, or at least 5%, or at least 10%, or at least 20%, or at least 50%, or at least 80%. In particularly preferred embodiments, the reduced cellular apoptosis is associated with reduced expression of pro-apoptotic initiators (BIM, PUMA), pro-apoptotic effectors (BAX, BAK), apoptogenic factors (SMAC, DIABLO, BID, BAD, etc.), or any combination thereof, by at least 1%, or at least 2%, or at least 5%, or at least 10%, or at least 20%, or at least 50%, or at least 80%. In preferred embodiments, the reduced cellular apoptosis is associated with increased anti-apoptotic marker expression, by at least 1%, or at least 2%, or at least 5%, or at least 10%, or at least 20%, or at least 50%, or at least 80%. In particularly preferred embodiments, the reduced cellular apoptosis is associated with increased expression of BCL-2, BCL-XL, BCL-W, A1/BFL-1, or MCL-1 by at least 1%, or at least 2%, or at least 5%, or at least 10%, or at least 20%, or at least 50%, or at least 80%.

In some embodiments, the engineered lung treated with the isolated mitochondria has increased glucose uptake and decreased lactate production in comparison to a corresponding engineered lung not treated with the isolated mitochondria. In preferred embodiments, the increased glucose uptake and decreased lactate production is associated with increased expression of HK, VDAC1, GLUT, AKT1, or any combination thereof, by at least 1%, or at least 2%, or at least 5%, or at least 10%, or at least 20%, or at least 50%, or at least 80%.

Non-limiting examples of repopulating cells are epithelial cells (e.g., type I alveolar cells, type II alveolar cells, small and large airway epithelial cells), endothelial cells (e.g., human pulmonary artery endothelial cells (HPAEC)), fibroblasts, progenitor cells (e.g., endothelial progenitor cells and mesenchymal stem cells), smooth muscle cells (e.g., pulmonary artery smooth muscle cells), immune cells, mesenchymal cells, pericytes, and any combination thereof.

Also disclosed herein is a method for improving the function of an engineered lung, the method comprising: (i) delivering isolated mitochondria to repopulating cells, and (ii) repopulating a decellularized scaffold lung in a bioreactor, chamber, or vessel with the repopulating cells treated with the isolated mitochondria to produce an engineered lung. Likewise, the method can comprise repopulating the decellularized scaffold lung using cells that have been treated with isolated mitochondria before, during, after, or combinations thereof the cells have been delivered to the decellularized scaffold. In some embodiments, the isolated mitochondria are isolated porcine mitochondria. In some embodiments, the isolated mitochondria are isolated human mitochondria allogeneic to the engineered lung. In some embodiments, the isolated mitochondria are isolated human mitochondria autologous to the engineered lung. In preferred embodiments, cells of the engineered lung treated with the isolated mitochondria have at least 1%, or at least 2%, or at least 5%, or at least 10%, or at least 20%, or at least 50%, or at least 100%, improvement in mitochondrial function in comparison to cells of a corresponding engineered lung not treated with the isolated mitochondria. In particularly preferred embodiments, the engineered lung treated with the isolated mitochondria has one or more improved cellular, organ, or tissue functions in comparison to a corresponding engineered lung not treated with the isolated mitochondria, wherein the one or more improved cellular, organ, or tissue functions are increased cell adherence to the scaffold, increased cell viability, reduced apoptosis, reduced cell damage, increased cell proliferation, increased cellular barrier function, reduced DNA damage, increased angiogenesis, improved blood vessel maintenance, reduced mitochondrial stress signaling, reduced reactive oxygen species production, or any combination thereof. In preferred embodiments, the engineered lung is an engineered human lung.

In some embodiments, the method further comprises the step of performing EVLP on the engineered lung by perfusing the engineered lung with a perfusate solution from a reservoir. In some embodiments, the perfusate solution is introduced into the engineered lung through a cannulated pulmonary artery. In some embodiments, the engineered lung is ventilated in the chamber or vessel through a cannulated trachea.

In some embodiments, the engineered lung treated with the isolated mitochondria has reduced inflammation and/or immune cell activation in comparison to a corresponding engineered lung not treated with the isolated mitochondria. In preferred embodiments, the reduced inflammation and/or immune cell activation is associated with reduced expression of MAPK14, JNK, or p53, by at least 1%, or at least 2%, or at least 5%, or at least 10%, or at least 20%, or at least 50%, or at least 80%. In preferred embodiments, the reduced inflammation and/or immune cell activation is associated with reduced expression of NF-κB, by at least 1%, or at least 2%, or at least 5%, or at least 10%, or at least 20%, or at least 50%, or at least 80%. In preferred embodiments, the reduced inflammation and/or immune cell activation is associated with reduced secretion of pro-inflammatory cytokines and chemokines such as MIP-1β (CCL4), PDGF-BB, RANTES (CCL5), soluble ICAM-1 (sICAM-1), M-CSF (CSF-1), IL-1β, IL-6, IL-8 (CXCL8), GDF-15, TGF-β1, and any combination thereof, by at least 1%, or at least 2%, or at least 5%, or at least 10%, or at least 20%, or at least 50%, or at least 80%. In preferred embodiments, the reduced inflammation and/or immune cell activation is associated with reduced expression of activation markers such as CD69, CD95, CD30, CD137, CD25 (IL2RA), CD38, CD154 (CD40L), and any combination thereof, by at least 1%, or at least 2%, or at least 5%, or at least 10%, or at least 20%, or at least 50%, or at least 80%. In preferred embodiments, the reduced inflammation and/or immune cell activation is associated with reduced expression or secretion of IL-2, IL-4, IL-5, IL-6, IL-9, IL-13, IL17, TNF-α, IFN-γ, or any combination thereof, by at least 1%, or at least 2%, or at least 5%, or at least 10%, or at least 20%, or at least 50%, or at least 80%.

In some embodiments, the engineered lung treated with the isolated mitochondria has reduced cellular apoptosis, increased cell viability, reduced mitochondrial stress signaling, and/or reduced cell damage in comparison to a corresponding engineered lung not treated with the isolated mitochondria. In preferred embodiments, the reduced cell damage is associated with reduced TLR9 expression, altered HO-1 expression, reduced cytosolic mtDNA, or any combination thereof, by at least 1%, or at least 2%, or at least 5%, or at least 10%, or at least 20%, or at least 50%, or at least 80%. In some embodiments, the altered HO-1 expression is increased HO-1 expression after cold exposure. In preferred embodiments, the reduced cellular apoptosis, increased cell viability, reduced mitochondrial stress signaling, and/or reduced cell damage is associated with reduced NF-κB, MAPK14, JNK, p53 expression, or any combination thereof, by at least 1%, or at least 2%, or at least 5%, or at least 10%, or at least 20%, or at least 50%, or at least 80%. In preferred embodiments, the reduced cellular apoptosis is associated with reduced pro-apoptotic marker expression, by at least 1%, or at least 2%, or at least 5%, or at least 10%, or at least 20%, or at least 50%, or at least 80%. In particularly preferred embodiments, the reduced cellular apoptosis is associated with reduced expression of pro-apoptotic initiators (BIM, PUMA), pro-apoptotic effectors (BAX, BAK), apoptogenic factors (SMAC, DIABLO, BID, BAD, etc.), or any combination thereof, by at least 1%, or at least 2%, or at least 5%, or at least 10%, or at least 20%, or at least 50%, or at least 80%. In preferred embodiments, the reduced cellular apoptosis is associated with increased anti-apoptotic marker expression, by at least 1%, or at least 2%, or at least 5%, or at least 10%, or at least 20%, or at least 50%, or at least 80%. In particularly preferred embodiments, the reduced cellular apoptosis is associated with increased expression of BCL-2, BCL-XL, BCL-W, A1/BF-L1, or MCL-1 by at least 1%, or at least 2%, or at least 5%, or at least 10%, or at least 20%, or at least 50%, or at least 80%.

In some embodiments, the engineered lung treated with the isolated mitochondria has increased glucose uptake and decreased lactate production in comparison to a corresponding engineered lung not treated with the isolated mitochondria. In preferred embodiments, the increased glucose uptake and decreased lactate production is associated with increased expression of HK, VDAC1, GLUT, AKT1, or any combination thereof, by at least 1%, or at least 2%, or at least 5%, or at least 10%, or at least 20%, or at least 50%, or at least 80%.

Also disclosed herein is a method for improving the function of an engineered kidney, the method comprising: (i) repopulating a decellularized scaffold kidney in a bioreactor, chamber, or vessel with repopulating cells to produce an engineered kidney, and (ii) delivering isolated mitochondria to the engineered kidney. Likewise, the method can comprise repopulating the decellularized scaffold kidney using cells that have been treated with isolated mitochondria before, during, after, or combinations thereof the cells have been delivered to the decellularized scaffold. In some embodiments, the isolated mitochondria are isolated porcine mitochondria. In some embodiments, the isolated mitochondria are isolated human mitochondria allogeneic to the engineered kidney. In some embodiments, the isolated mitochondria are isolated human mitochondria autologous to the engineered kidney. In preferred embodiments, cells of the engineered kidney treated with the isolated mitochondria have at least 1%, or at least 2%, or at least 5%, or at least 10%, or at least 20%, or at least 50%, or at least 100%, improvement in mitochondrial function in comparison to cells of a corresponding engineered kidney not treated with the isolated mitochondria. In particularly preferred embodiments, the engineered kidney treated with the isolated mitochondria has one or more improved cellular, organ, or tissue functions in comparison to a corresponding engineered kidney not treated with the isolated mitochondria, wherein the one or more improved cellular, organ, or tissue functions are increased cell adherence to the scaffold, increased cell viability, reduced apoptosis, reduced cell damage, increased cell proliferation, increased cellular barrier function, reduced DNA damage, increased angiogenesis, improved blood vessel maintenance, reduced mitochondrial stress signaling, reduced reactive oxygen species production, or any combination thereof. In preferred embodiments, the engineered kidney is an engineered human kidney.

In some embodiments, the isolated mitochondria are delivered to the engineered kidney after the step of repopulating the decellularized scaffold kidney. In other embodiments, the isolated mitochondria are delivered to the engineered kidney during the step of repopulating the decellularized scaffold kidney. In preferred embodiments, the isolated mitochondria are delivered to the engineered kidney together with the repopulating cells in the bioreactor, chamber, or vessel during the step of repopulating the decellularized scaffold kidney. In particularly preferred embodiments, the isolated mitochondria are delivered to the engineered kidney intravenously or intra-arterially.

In some embodiments, the engineered kidney treated with the isolated mitochondria has reduced inflammation and/or immune cell activation in comparison to a corresponding engineered kidney not treated with the isolated mitochondria. In preferred embodiments, the reduced inflammation and/or immune cell activation is associated with reduced expression of MAPK14, JNK, or p53, by at least 1%, or at least 2%, or at least 5%, or at least 10%, or at least 20%, or at least 50%, or at least 80%. In preferred embodiments, the reduced inflammation and/or immune cell activation is associated with reduced expression of NF-κB, by at least 1%, or at least 2%, or at least 5%, or at least 10%, or at least 20%, or at least 50%, or at least 80%. In preferred embodiments, the reduced inflammation and/or immune cell activation is associated with reduced secretion of pro-inflammatory cytokines and chemokines such as MIP-1β (CCL4), PDGF-BB, RANTES (CCL5), soluble ICAM-1 (sICAM-1), M-CSF (CSF-1), IL-1β, IL-6, IL-8 (CXCL8), GDF-15, TGF-β1, and any combination thereof, by at least 1%, or at least 2%, or at least 5%, or at least 10%, or at least 20%, or at least 50%, or at least 80%. In preferred embodiments, the reduced inflammation and/or immune cell activation is associated with reduced expression of activation markers such as CD69, CD95, CD30, CD137, CD25 (IL2RA), CD38, CD154 (CD40L), and any combination thereof, by at least 1%, or at least 2%, or at least 5%, or at least 10%, or at least 20%, or at least 50%, or at least 80%. In preferred embodiments, the reduced inflammation and/or immune cell activation is associated with reduced expression or secretion of IL-2, IL-4, IL-5, IL-6, IL-9, IL-13, IL17, TNF-α, IFN-γ, or any combination thereof, by at least 1%, or at least 2%, or at least 5%, or at least 10%, or at least 20%, or at least 50%, or at least 80%.

In some embodiments, the engineered kidney treated with the isolated mitochondria has reduced cellular apoptosis, increased cell viability, reduced mitochondrial stress signaling, and/or reduced cell damage in comparison to a corresponding engineered kidney not treated with the isolated mitochondria. In preferred embodiments, the reduced cell damage is associated with reduced TLR9 expression, altered HO-1 expression, reduced cytosolic mtDNA, or any combination thereof, by at least 1%, or at least 2%, or at least 5%, or at least 10%, or at least 20%, or at least 50%, or at least 80%. In some embodiments, the altered HO-1 expression is increased HO-1 expression after cold exposure. In preferred embodiments, the reduced cellular apoptosis, increased cell viability, reduced mitochondrial stress signaling, and/or reduced cell damage is associated with reduced NF-κB, MAPK14, JNK, p53 expression, or any combination thereof, by at least 1%, or at least 2%, or at least 5%, or at least 10%, or at least 20%, or at least 50%, or at least 80%. In preferred embodiments, the reduced cellular apoptosis is associated with reduced pro-apoptotic marker expression, by at least 1%, or at least 2%, or at least 5%, or at least 10%, or at least 20%, or at least 50%, or at least 80%. In particularly preferred embodiments, the reduced cellular apoptosis is associated with reduced expression of pro-apoptotic initiators (BIM, PUMA), pro-apoptotic effectors (BAX, BAK), apoptogenic factors (SMAC, DIABLO, BID, BAD, etc.), or any combination thereof, by at least 1%, or at least 2%, or at least 5%, or at least 10%, or at least 20%, or at least 50%, or at least 80%. In preferred embodiments, the reduced cellular apoptosis is associated with increased anti-apoptotic marker expression, by at least 1%, or at least 2%, or at least 5%, or at least 10%, or at least 20%, or at least 50%, or at least 80%. In particularly preferred embodiments, the reduced cellular apoptosis is associated with increased expression of BCL-2, BCL-XL, BCL-W, A1/BFL-1, or MCL-1 by at least 1%, or at least 2%, or at least 5%, or at least 10%, or at least 20%, or at least 50%, or at least 80%.

In some embodiments, the engineered kidney treated with the isolated mitochondria has increased glucose uptake and decreased lactate production in comparison to a corresponding engineered kidney not treated with the isolated mitochondria. In preferred embodiments, the increased glucose uptake and decreased lactate production is associated with increased expression of HK, VDAC1, GLUT, AKT1, or any combination thereof, by at least 1%, or at least 2%, or at least 5%, or at least 10%, or at least 20%, or at least 50%, or at least 80%.

Non-limiting examples of repopulating cells are epithelial cells (e.g., type I alveolar cells, type II alveolar cells, small and large airway epithelial cells), endothelial cells (e.g., human pulmonary artery endothelial cells (HPAEC)), fibroblasts, progenitor cells (e.g., endothelial progenitor cells and mesenchymal stem cells), smooth muscle cells (e.g., pulmonary artery smooth muscle cells), immune cells, mesenchymal cells, pericytes, and any combination thereof.

Also disclosed herein is a method for improving the function of an engineered kidney, the method comprising: (i) delivering isolated mitochondria to repopulating cells, and (ii) repopulating a decellularized scaffold kidney in a bioreactor, chamber, or vessel with the repopulating cells treated with the isolated mitochondria to produce an engineered kidney. In some embodiments, the isolated mitochondria are isolated porcine mitochondria. In some embodiments, the isolated mitochondria are isolated human mitochondria allogeneic to the engineered kidney. In some embodiments, the isolated mitochondria are isolated human mitochondria autologous to the engineered kidney. In preferred embodiments, cells of the engineered kidney treated with the isolated mitochondria have at least 1%, or at least 2%, or at least 5%, or at least 10%, or at least 20%, or at least 50%, or at least 100%, improvement in mitochondrial function in comparison to cells of a corresponding engineered kidney not treated with the isolated mitochondria. In particularly preferred embodiments, the engineered kidney treated with the isolated mitochondria has one or more improved cellular, organ, or tissue functions in comparison to a corresponding engineered kidney not treated with the isolated mitochondria, wherein the one or more improved cellular, organ, or tissue functions are increased cell adherence to the scaffold, increased cell viability, reduced apoptosis, reduced cell damage, increased cell proliferation, increased cellular barrier function, reduced DNA damage, increased angiogenesis, improved blood vessel maintenance, reduced mitochondrial stress signaling, reduced reactive oxygen species production, or any combination thereof. In preferred embodiments, the engineered kidney is an engineered human kidney.

In some embodiments, the engineered kidney treated with the isolated mitochondria has reduced inflammation and/or immune cell activation in comparison to a corresponding engineered kidney not treated with the isolated mitochondria. In preferred embodiments, the reduced inflammation and/or immune cell activation is associated with reduced expression of MAPK14, JNK, or p53, by at least 1%, or at least 2%, or at least 5%, or at least 10%, or at least 20%, or at least 50%, or at least 80%. In preferred embodiments, the reduced inflammation and/or immune cell activation is associated with reduced expression of NF-κB, by at least 1%, or at least 2%, or at least 5%, or at least 10%, or at least 20%, or at least 50%, or at least 80%. In preferred embodiments, the reduced inflammation and/or immune cell activation is associated with reduced secretion of pro-inflammatory cytokines and chemokines such as MIP-1β (CCL4), PDGF-BB, RANTES (CCL5), soluble ICAM-1 (sICAM-1), M-CSF (CSF-1), IL-1β, IL-6, IL-8 (CXCL8), GDF-15, TGF-β1, and any combination thereof, by at least 1%, or at least 2%, or at least 5%, or at least 10%, or at least 20%, or at least 50%, or at least 80%. In preferred embodiments, the reduced inflammation and/or immune cell activation is associated with reduced expression of activation markers such as CD69, CD95, CD30, CD137, CD25 (IL2RA), CD38, CD154 (CD40L), and any combination thereof, by at least 1%, or at least 2%, or at least 5%, or at least 10%, or at least 20%, or at least 50%, or at least 80%. In preferred embodiments, the reduced inflammation and/or immune cell activation is associated with reduced expression or secretion of IL-2, IL-4, IL-5, IL-6, IL-9, IL-13, IL17, TNF-α, IFN-γ, or any combination thereof, by at least 1%, or at least 2%, or at least 5%, or at least 10%, or at least 20%, or at least 50%, or at least 80%.

In some embodiments, the engineered kidney treated with the isolated mitochondria has reduced cellular apoptosis, increased cell viability, reduced mitochondrial stress signaling, and/or reduced cell damage in comparison to a corresponding engineered kidney not treated with the isolated mitochondria. In preferred embodiments, the reduced cell damage is associated with reduced TLR9 expression, altered HO-1 expression, reduced cytosolic mtDNA, or any combination thereof, by at least 1%, or at least 2%, or at least 5%, or at least 10%, or at least 20%, or at least 50%, or at least 80%. In some embodiments, the altered HO-1 expression is increased HO-1 expression after cold exposure. In preferred embodiments, the reduced cellular apoptosis, increased cell viability, reduced mitochondrial stress signaling, and/or reduced cell damage is associated with reduced NF-κB, MAPK14, JNK, p53 expression, or any combination thereof, by at least 1%, or at least 2%, or at least 5%, or at least 10%, or at least 20%, or at least 50%, or at least 80%. In preferred embodiments, the reduced cellular apoptosis is associated with reduced pro-apoptotic marker expression, by at least 1%, or at least 2%, or at least 5%, or at least 10%, or at least 20%, or at least 50%, or at least 80%. In particularly preferred embodiments, the reduced cellular apoptosis is associated with reduced expression of pro-apoptotic initiators (BIM, PUMA), pro-apoptotic effectors (BAX, BAK), apoptogenic factors (SMAC, DIABLO, BID, BAD, etc.), or any combination thereof, by at least 1%, or at least 2%, or at least 5%, or at least 10%, or at least 20%, or at least 50%, or at least 80%. In preferred embodiments, the reduced cellular apoptosis is associated with increased anti-apoptotic marker expression, by at least 1%, or at least 2%, or at least 5%, or at least 10%, or at least 20%, or at least 50%, or at least 80%. In particularly preferred embodiments, the reduced cellular apoptosis is associated with increased expression of BCL-2, BCL-XL, BCL-W, A1/BFL-1, or MCL-1 by at least 1%, or at least 2%, or at least 5%, or at least 10%, or at least 20%, or at least 50%, or at least 80%.

In some embodiments, the engineered kidney treated with the isolated mitochondria has increased glucose uptake and decreased lactate production in comparison to a corresponding engineered kidney not treated with the isolated mitochondria. In preferred embodiments, the increased glucose uptake and decreased lactate production is associated with increased expression of HK, VDAC1, GLUT, AKT1, or any combination thereof, by at least 1%, or at least 2%, or at least 5%, or at least 10%, or at least 20%, or at least 50%, or at least 80%.

In some embodiments of the present methods, the engineered organ, tissue, kidney, or lung is generated using an artificial organ or tissue matrix. Methods and materials for a preparing an artificial organ or tissue matrix are known in the art. Any appropriate materials can be used to prepare such a matrix. In a preferred embodiment, an artificial organ or tissue matrix can be a scaffold developed from porous materials such as, for example, polyglycolic acid, Pluronic F-127 (PF-127), Gelfoam sponge, collagen-glycosaminoglycan (GAG), fibrinogen-fibronectin-vitronectin hydrogel (FFVH), and elastin. See, e.g., Ingenito et al., J Tissue Eng Regen Med. 2009 Dec. 17; Hoganson et al., Pediatric Research, 2008, 63(5):520-526; Chen et al., Tissue Eng. 2005 September-October; 11(9-10): 1436-48. In some cases, an artificial organ or tissue matrix can have porous structures similar to alveolar units. See Andrade et al., Am J Physiol Lung Cell Mol Physiol. 2007, 292(2):L510-8. In some cases, an implanted artificial organ or tissue matrix can express organ-specific markers (e.g., lung-specific markers for Clara cells (i.e., club cells), pneumocytes, and respiratory epithelium). In some cases, an implanted artificial organ or tissue matrix can organize into identifiable structures (e.g., structures similar to alveoli and terminal bronchi in an artificial lung matrix). For example, an implanted artificial lung matrix made using FFVH can promote cell attachment, spreading and extracellular matrix expression in vitro and apparent engraftment in vivo, with evidence of trophic effects on the surrounding tissue. See Ingenito et al., supra. See also U.S. Pat. Nos. 7,662,409 and 6,087,552; United States Patent Publication Nos. 2010/0034791; 2009/0075282; 2009/0035855; 2008/0292677; 2008/0131473; 2007/0059293; 2005/0196423; 2003/0166274; 2003/0129751; 2002/0182261; 2002/0182241; and 2002/0172705. In preferred embodiments, the artificial organ or tissue matrix is infused with isolated mitochondria prior to the seeding of populating cells to support the metabolism, attachment, and viability of the populating cells.

In some embodiments, the artificial organ or tissue matrix is generated by bioprinting. See, e.g., Murphy, S. V. and Atala, A., Nat Biotechnol. 2004, 32(8):773-85. In preferred embodiments, the populating cells and the artificial organ or tissue matrix are printed concurrently to form a populated organ or tissue matrix. In preferred embodiments, isolated mitochondria are delivered with the populating cells and/or matrix during printing in order to support cell viability during the initial period of bioprinting. In preferred embodiments, the bioprinted organ or tissue matrix is infused with isolated mitochondria prior to the seeding of populating cells to support the metabolism, attachment, and viability of the populating cells.

In some embodiments of the present methods, cadaveric organs are prepared and maintained for use in transplantation. Methods and materials to isolate donor organs (e.g., lungs and kidneys) from human and animal donors are known in the art. For example, described in Pasque, M. et al., J Thorac Cardiovasc Surg. 2010, 139(1):13-7 and Bribriesco A. et al., Front Biosci 2013, 5:266-72. Any appropriate method to isolate these can be used. These donor organs can be maintained using bioreactors, chambers, or vessels for a time sufficient to prepare a recipient for transplant, for a time sufficient to transport the organ to the recipient, or for a time sufficient to maintain the organ under conditions that facilitate the repair of the entire organ or portion thereof so that it is suitable for implantation.

In some embodiments, donor organs from organ donors can be modified to remove endothelial lining and subsequently reseeded with recipient-derived endothelial cells to minimize immunogenicity. For example, this can be accomplished by osmotic challenge via perfusion with deionized water, perfusion with low detergent concentrations such as 0.05% Polidocanol, or perfusion with enzyme solutions such as DNase, or collagenase. Donor organs found unsuitable for immediate transplantation due to infection, physical damage such as trauma, or ischemic damage due to prolonged hypoperfusion, or damage due to donor conditions such as brain death can be repaired using the devices and methods described herein (e.g., by mounting, perfusing, and repairing using antibiotics, cells, growth factor stimulation, and anti-inflammatory treatment). Animal-derived organs can be rendered less immunogenic by genetic and cellular modification.

In some cases, donor lungs may exhibit evidence of damage resulting from a variety of factors, e.g., quality of the donor lung, the type of preservation solution, length of time between harvest and culture, and so forth. In order to reduce and/or eliminate the degree of damage the donor lungs and/or portions thereof can be mounted, e.g., on devices described herein, and ventilated liquid and/or dry ventilation. In an example, air is perfused through the tracheal line, while the ventricular and/or arterial lines are perfused with a solution that mimics physiologic parameters, e.g., physiologic saline solution, blood containing solution, Steen solution, Perfadex and/or a preservation solution. The donor lungs may remain mounted until the donor lungs are needed for transplant and/or until the damaged donor lungs exhibit re-epithelialization and exhibit improved lung function (e.g., improved endothelial barrier function, improved vascular flow rate, decreased pulmonary edema, and/or improved ratio of arterial oxygen partial pressure to fractional inspired oxygen (PaO2/FiO2)). These perfusion methods can be combined with the cellular seeding methods, as described below.

In some of the methods described herein, a lung or kidney tissue matrix, e.g., decellularized lung or kidney tissue matrix or artificial lung or kidney matrix, is seeded with cells, e.g., differentiated or regenerative cells. Any appropriate regenerative cell type, such as naive or undifferentiated cell types, can be used to seed the lung or kidney tissue matrix. The cells may be seeded at a variety of stages including, but not limited to, stem cell stage (e.g., after induction), progenitor cell stage, hemangioblast stage, or differentiated stage (e.g., CD 31+, CD144+). As used herein, regenerative cells can include, without limitation, progenitor cells, precursor cells, and “adult’-derived stem cells including umbilical cord cells (e.g., human umbilical vein endothelial cells) and fetal stem cells. Regenerative cells also can include differentiated or committed cell types. Stem cells appropriate for the methods and materials provided herein can include human induced pluripotent stem cells (iPSC) (e.g., undifferentiated, differentiated endoderm, anteriolized endoderm, TTF-1 positive lung progenitors), human mesenchymal stem cells, human umbilical vein endothelial cells, multipotent adult progenitor cells (MAPC), iPS derived mesenchymal cells, or embryonic stem cells. In some cases, regenerative cells derived from other tissues also can be used. For example, regenerative cells derived from skin, bone, muscle, heart, bone marrow, synovium, Wharton's jelly, placenta, foreskin, or adipose tissue can be used to develop stem cell-seeded tissue matrices.

In some cases, a lung or kidney tissue matrix provided herein can be alternatively or further seeded with differentiated cell types such as (preferably human) epithelial cells and endothelial cells. For example, a lung matrix can be seeded with endothelial cells via the vasculature (e.g., through the arterial line or the venous line), and seeded with epithelial cells via the airway (e.g., through the tracheal line). The lung or kidney matrix can also be seeded with one or more cell types (e.g., one or more types of epithelial and mesenchymal cells, adult peripheral blood derived epithelial cells, cord blood-derived epithelial cells, iPS derived epithelial cells, progenitor stage cells (e.g., smooth muscle), adult lung derived cell mixture (e.g., rat human), commercially available small airway epithelial cells or alveolar epithelial cells, Embryonic Stem (ES) cell-derived epithelial cells, and/or human umbilical vein endothelial cells (HUVEC). Any type of appropriate commercially available media and/or media kits may be used for the seeding and culture of cells. For example, SAGM media may be used for small airway cells (e.g., SAGM BulletKit by Lonza) and EGM-2 kits may be used for endothelial cells (e.g., EGM-2 BulletKit by Lonza). Media customized to the seeded endothelial cell type may be used (e.g., by increasing or decreasing growth factors such as VEGF) as described in, for example, Brudno, Y. et al., Biomaterials 2013, 34:9201-9. In the case of endothelial cells, a sequence of different media compositions may be used to induce different phases of seeding, expansion, engraftment, and maturation of cells. For example, in a first phase, a cell seeded constructs may be perfused with an ‘angiogenic media’ for 2-30 days to increase endothelial cell expansion, migration, and metabolism. This media is characterized by high concentration of cytokines, e.g., VEGF at 5-100 ng/ml and bFGF at 5-100 ng/ml, and the presence of phorbol myristate acetate (PMA), e.g., 5-100 ng/ml PMA, which activates the angiogenic pathway through activation of protein kinase C, and Ang-1, which stimulates endothelial cell sprouting. In a second phase, a cell seeded construct can then be perfused with ‘tightening media’ that supports endothelial maturation and the formation of tight junctions. Tightening media has lower levels of cytokines, with the same basic composition as the angiogenic media but with decreased levels of VEGF, bFGF and PMA (0.1-5 ng/ml VEGF, FGF, and PMA). Hydrocortisone, which promotes tight junction formation and has been shown to reduce pulmonary edema, can be further added to the tightening media to promote vascular maturation. Further promaturation factors such as PDGF and Ang-2 may be added to the tightening media to enhance vessel formation. Concentrations of these factors may be titrated to support different vessel sizes. Media changes can be performed gradually to avoid detrimental effects of sudden cytokine changes. Similar to endothelial cell supporting media, sequential media changes can be used to guide epithelial cell fate. Initial media may contain, for example, Activin A at 10-200 ng/ml and Pi3K inhibitors such as ZSTK 474 at 0.01-1 uM to induce definite endoderm, subsequently TGF-beta inhibitors such as A-8301 at 01-10 uM and BMP4 antagonists such as DMH-1 at 0.05-1 uM to induce anteriorized endoderm, and finally BMP4 at 1-100 ug/ml, FGF2 at 10-500 ng/ml, GSK-3beta inhibitor such as CHIR 99021 at 10-500 nM, a PI3K inhibitor such as PIK-75 at 1-100 nM and methotrexate at 1-100 nM to induce the generation of lung progenitor cells.

Any appropriate method for isolating and collecting cells for seeding can be used. For example, induced pluripotent stem cells generally can be obtained from somatic cells “reprogrammed” to a pluripotent state by the ectopic expression of transcription factors such as Oct4, Sox2, Klf4, c-MYC, Nanog, and Lin28. See Takahashi et al., Cell 2007, 131:861-72 (2007); Park et al, Nature 451:141-146 (2008); Yu et al, Science 318: 1917-20; Zhu et al., Cell Stem Cell 2010, 7:651-5; and Li et al., Cell Res. 2011, 21:196-204; Malik and Rao, Methods Mol Biol. 2013; 997:23-33; Okano et al, Circ Res. 2013 Feb. 1; 112(3):523-33; Lin and Ying, Methods Mol Biol. 2013, 936:295-312. Peripheral blood-derived mononuclear cells can be isolated from patient blood samples and used to generate induced pluripotent stem cells. In other examples, induced pluripotent stem cells can be obtained by reprograming with constructs optimized for high co-expression of Oct4, Sox2, Klf4, c-MYC in conjunction with small molecule such as transforming growth factor β (SB431542), MEK/ERK (PD0325901) and Rho-kinase signaling (Thiazovivin). See GroB et al., Curr Mol Med. 2013, 13:765-76 and Hou et al., Science 2013, 341:651-4. Methods for generating endothelial cells from stem cells are reviewed in Reed et al., Br J Clin Pharmacol. 2013, 75(4):897-906. Cord blood stem cells can be isolated from fresh or frozen umbilical cord blood. Mesenchymal stem cells can be isolated from, for example, raw unpurified bone marrow or ficoll-purified bone marrow. Epithelial and endothelial cells can be isolated and collected from living or cadaveric donors, e.g., from the subject who will be receiving the bioartificial kidney or lung, according to methods known in the art. For example, epithelial cells can be obtained from a skin tissue sample (e.g., a punch biopsy), and endothelial cells can be obtained from a vascular tissue sample. In some embodiments, proteolytic enzymes are perfused into the tissue sample through a catheter placed in the vasculature. Portions of the enzymatically treated tissue can be subjected to further enzymatic and mechanical disruption. The mixture of cells obtained in this manner can be separated to purify epithelial and endothelial cells. In some cases, flow cytometry-based methods (e.g., fluorescence-activated cell sorting) can be used to sort cells based on the presence or absence of specific cell surface markers. Furthermore, kidney or lung cells (e.g., epithelial, mesenchymal, and endothelial) can be obtained from kidney or lung biopsies, which can be obtained, for example, via transbronchial and endobronchial biopsies or via surgical biopsies of kidney or lung tissue. In cases where non-autologous cells are used, the selection of immune type-matched cells should be considered, so that the organ or tissue will not be rejected when implanted into a subject.

In some cases, a decellularized or artificial kidney or lung tissue matrix, as provided herein, can be seeded with the cell types by perfusion seeding. For example, a flow perfusion system can be used to seed the decellularized kidney or lung tissue matrix via the vascular system preserved in the tissue matrix (e.g., through the arterial line). In some cases, automated flow perfusion systems can be used under the appropriate conditions. Such perfusion seeding methods can improve seeding efficiencies and provide more uniform distribution of cells throughout the composition. Quantitative biochemical and image analysis techniques can be used to assess the distribution of seeded cells following either static or perfusion seeding methods.

In some cases, a tissue matrix can be impregnated with one or more growth factors to stimulate differentiation of the seeded regenerative cells. For example, a tissue matrix can be impregnated with growth factors appropriate for the methods and materials provided herein, for example, vascular endothelial growth factor (VEGF), TGF-β growth factors, bone morphogenetic proteins (e.g., BMP-1, BMP-4), platelet-derived growth factor (PDGF), basic fibroblast growth factor (b-FGF), e.g., FGF-10, insulin-like growth factor (IGF), epidermal growth factor (EGF), or growth differentiation factor-5 (GDF-5). See, e.g., Desai and Cardoso, Respire. Res. 2002, 3:2. These growth factors can be encapsulated to control temporal release. Different parts of the scaffold can be enhanced with different growth factors to add spatial control of growth factor stimulation. In some cases, the tissue matrix can be impregnated with extracellular matrix components (e.g., laminin, fibronectin, collagen, elastin) prior to the seeding of regenerative cells to support the attachment and growth of regenerative cells. In some cases, the tissue matrix can be impregnated with isolated mitochondria prior to the seeding of regenerative cells to support the metabolism, attachment, and viability of the regenerative cells.

Seeded tissue matrices can be incubated for a period of time (e.g., from several hours to about 14 days or more) post-seeding to improve fixation and penetration of the cells in the tissue matrix. The seeded tissue matrix can be maintained under conditions in which at least some of the regenerative cells can multiply and/or differentiate within and on the acellular tissue matrix. Such conditions can include, without limitation, the appropriate temperature (35-38 degree centigrade) and/or pressure (e.g., atmospheric), electrical and/or mechanical activity (e.g., ventilation via positive or negative pressure with positive end expiratory pressure from 1-20 cmH₂O, mean airway pressure from 5-50 cmH₂O, and peak inspiratory pressure from 5-65cmH₂O), the appropriate amounts of fluid, e.g., O₂ (1-100% FiO₂) and/or CO₂ (0-10% FiCO₂), an appropriate amount of humidity (10-100%), and sterile or near-sterile conditions. Such conditions can also include wet ventilation, wet to dry ventilation and dry ventilation. In some cases, nutritional supplements (e.g., nutrients and/or a carbon source such as glucose), exogenous hormones, or growth factors can be added to the seeded tissue matrix. Histology and cell staining can be performed to assay for seeded cell propagation. Any appropriate method can be performed to assay for seeded cell differentiation.

Thus, the methods described herein can be used to generate a transplantable bioartificial organ or tissue, e.g., an artificial kidney or lung for transplanting into a human subject. A transplantable organ or tissue will preferably retain a sufficiently intact vasculature that can be connected to the patient's vascular system.

### IV. Methods of Treating a Subject

Disclosed herein is a method for treating a lung disease or disorder of a subject in need thereof or for improving the function of a donor lung prior to or after transplantation, the method comprising administering to the subject or donor lung a pharmaceutical composition comprising a mesenchymal stem cell or endothelial progenitor cell that has been pre-treated with isolated mitochondria, or extracellular vesicles isolated from the mesenchymal stem cell or endothelial progenitor cell. In some embodiments, the isolated mitochondria are isolated porcine mitochondria. In some embodiments, the isolated mitochondria are isolated human mitochondria allogeneic to the subject or donor lung. In some embodiments, the isolated mitochondria are isolated human mitochondria autologous to the subject or donor lung. In some embodiments, the composition is administered to the subject by inhalation. In some embodiments, the composition is administered to the subject or donor lung through the lung airway. In other embodiments, the composition is administered to the subject or donor lung by injection (e.g., intravenous, subcutaneous, intraperitoneal, and intramusclular injection). In some embodiments, the composition further comprises at least one pharmaceutically acceptable carrier or excipient. In some embodiments, the composition further comprises at least one active ingredient. In preferred embodiments, the subject is a human subject.

Non-limiting examples of pharmaceutically acceptable carriers or excipients are respiration buffers (e.g., a buffer containing sucrose, glutamate, malate, succinate, and ADP); extracellular matrix components (e.g., laminin, fibronectin, collage, elastin); organ or tissue preservation solutions (e.g., Euro-Collins solution); isotonic saline; water; balanced salt solutions; aqueous dextrose; polyols (e.g., glycerol, propylene glycol, liquid polyethylene glycol, and the like); and vegetable oils. One skilled in the art may refer to the reference handbook “Handbook of Pharmaceutical Excipients”, American Pharmaceutical Association, Pharmaceutical Press; 6th revised edition, 2009). One skilled in the art may moreover select the carrier or excipient from carriers and excipients for pharmaceutical use known for being adapted to the preparation of compositions intended for injection or inhalation. Pharmaceutical forms suitable for injectable use include sterile aqueous solutions or dispersions and sterile powders for the extemporaneous preparation of sterile injectable solutions or dispersions. In all cases the form can be fluid to the extent that easy syringeability exists. If needed, various antibacterial and antifungal agents can be used, for example, parabens, chlorobutanol, phenol, sorbic acid, thimerosal, and the like. In many cases, it will be preferable to include isotonic agents, for example, sugars or sodium chloride.

Non-limiting examples of active ingredients are treprostinil, anti-oxidants, anti-histaminic agents, immuno-modulators, biological additives, analgesics, anesthetic agents, antibiotics, antifungal agents, UNEX-42, and anti-inflammatory agents. In certain embodiments, the active ingredient is a pharmaceutical active ingredient and exerts a therapeutic effect.

Also disclosed herein is a method for treating a lung disease or disorder in a subject in need thereof or for improving the function of a donor lung prior to or after transplantation, the method comprising administering to the subject or donor lung (A) a mesenchymal stem cell or endothelial progenitor cell, or extracellular vesicles isolated from the mesenchymal stem cell or endothelial progenitor cell, and (B) isolated mitochondria, wherein (A) and (B) are comprised in a single pharmaceutical composition or two separate pharmaceutical compositions. In some embodiments, the isolated mitochondria are isolated porcine mitochondria. In some embodiments, the isolated mitochondria are isolated human mitochondria allogeneic to the subject or donor lung. In some embodiments, the isolated mitochondria are isolated human mitochondria autologous to the subject or donor lung. In some embodiments, the composition is administered to the subject by inhalation. In other embodiments, the composition is administered to the subject or donor lung through the lung airway. In other embodiments, the composition is administered to the subject or donor lung by injection (e.g., intravenous, subcutaneous, intraperitoneal, intramuscular). In some embodiments, the composition further comprises at least one pharmaceutically acceptable carrier or excipient. In some embodiments, the composition further comprises at least one active ingredient. In preferred embodiments, the subject is a human subject.

Also disclosed herein is a method for treating a lung disease or disorder in a subject in need thereof, the method comprising: (i) administering a therapeutically effective amount of a composition comprising isolated mitochondria to the subject, and (ii) administering a therapeutically effective amount of a medication for treating the lung disease or disorder, wherein the composition is administered to the subject before, concurrently with, or after the administration of the medication for treating the lung disease or disorder. In some embodiments, the isolated mitochondria are isolated porcine mitochondria. In some embodiments, the isolated mitochondria are isolated human mitochondria allogeneic to the subject. In some embodiments, the isolated mitochondria are isolated human mitochondria autologous to the subject. In some embodiments, the composition is administered to the subject by inhalation. In other embodiments, the composition is administered to the subject by injection. In some embodiments, the composition further comprises at least one pharmaceutically acceptable carrier or excipient. In some embodiments, the composition further comprises at least one active ingredient. In preferred embodiments, the subject is a human subject.

Non-limiting examples of pulmonary diseases and disorders are pulmonary hypertension, bronchopulmonary dysplasia (BPD), lung fibrosis, asthma, sleep-disordered breathing, or chronic obstructive pulmonary disease (COPD).

Non-limiting examples of pulmonary hypertension are pulmonary hypertension due to COPD, chronic thromboembolic pulmonary hypertension (CTEPH), pulmonary arterial hypertension (PAH), pulmonary veno-occlusive disease (PVOD), pulmonary capillary hemangiomatosis (PCH), persistent pulmonary hypertension of the newborn, BPD-induced pulmonary hypertension, pulmonary hypertension secondary to left heart disease, pulmonary hypertension due to lung disease, chronic hypoxia, chronic arterial obstruction, or pulmonary hypertension with unclear or multifactorial mechanisms.

Non-limiting examples of medications for treating a lung disease or disorder, such as pulmonary hypertension, are treprostinil, epoprostenol, iloprost, bosentan, ambrisentan, macitentan, and sildenafil.

Also disclosed herein is a method for treating pulmonary hypertension in a subject in need thereof, the method comprising: (i) administering a therapeutically effective amount of a composition comprising isolated mitochondria to the subject, and (ii) administering a therapeutically effective amount of treprostinil, wherein the composition is administered to the subject before, concurrently with, or after the administration of treprostinil. In some embodiments, the isolated mitochondria are isolated porcine mitochondria. In some embodiments, the isolated mitochondria are isolated human mitochondria allogeneic to the subject. In some embodiments, the isolated mitochondria are isolated human mitochondria autologous to the subject. In some embodiments, the composition is administered to the subject by inhalation. In other embodiments, the composition is administered to the subject by injection. In some embodiments, the composition further comprises at least one pharmaceutically acceptable carrier or excipient. In some embodiments, the composition further comprises at least one active ingredient. In preferred embodiments, the subject is a human subject.

UNEX-42 is a preparation of extracellular vesicles that are secreted from human mesenchymal stem cells. Also disclosed herein is a method for treating a lung disease or disorder of a subject in need thereof or for improving the function of a donor lung prior to or after transplantation, the method comprising: (i) administering a therapeutically effective amount of a composition comprising isolated mitochondria to the subject or donor lung, and (ii) administering a therapeutically effective amount of UNEX-42 to the subject or donor lung, wherein the composition is administered to the subject or donor lung before, concurrently with, or after the administration of UNEX-42. In some embodiments, the isolated mitochondria are isolated porcine mitochondria. In some embodiments, the isolated mitochondria are isolated human mitochondria allogeneic to the subject. In some embodiments, the isolated mitochondria are isolated human mitochondria autologous to the subject. In some embodiments, the composition is administered to the subject by inhalation. In other embodiments, the composition is administered to the subject or donor lung through the lung airway. In other embodiments, the composition is administered to the subject or donor lung by injection. In some embodiments, the composition further comprises at least one pharmaceutically acceptable carrier or excipient. In some embodiments, the composition further comprises at least one active ingredient. In preferred embodiments, the subject is a human subject.

Also disclosed herein is a method for treating a lung disease or disorder in a subject in need thereof or for improving the function of a donor lung prior to or after transplantation, the method comprising: (i) administering a therapeutically effective amount of a composition comprising isolated mitochondria to the subject or donor lung, and (ii) administering a therapeutically effective amount of an anti-oxidant to the subject or donor lung, wherein the composition is administered to the subject or donor lung before, concurrently with, or after the administration of the anti-oxidant. In some embodiments, the isolated mitochondria are isolated porcine mitochondria. In some embodiments, the isolated mitochondria are isolated human mitochondria allogeneic to the subject. In some embodiments, the isolated mitochondria are isolated human mitochondria autologous to the subject. In some embodiments, the anti-oxidant is n-acetylcysteine, tempol, or resveratrol. In some embodiments, the anti-oxidant is administered to the subject or donor lung concurrently with and as part of the composition comprising isolated mitochondria. In some embodiments, the composition is administered to the subject by inhalation. In other embodiments, the composition is administered to the subject or donor lung through the lung airway. In other embodiments, the composition is administered to the subject or donor lung by injection. In some embodiments, the composition further comprises at least one pharmaceutically acceptable carrier or excipient. In some embodiments, the composition further comprises at least one active ingredient. In preferred embodiments, the subject is a human subject.

Also disclosed herein is a method for treating an acute exacerbation of a lung disease or disorder in a subject, the method comprising administering an effective amount of a composition comprising isolated mitochondria to the subject for rescue therapy. In some embodiments, the isolated mitochondria are isolated porcine mitochondria. In some embodiments, the isolated mitochondria are isolated human mitochondria allogeneic to the subject. In some embodiments, the isolated mitochondria are isolated human mitochondria autologous to the subject. In preferred embodiments, the lung disease or disorder is pulmonary hypertension, asthma, sleep-disordered breathing, BPD, COPD, or lung fibrosis. In some embodiments, the pulmonary hypertension is pulmonary hypertension of the newborn, BPD-induced pulmonary hypertension, pulmonary hypertension secondary to left heart disease, pulmonary hypertension due to lung disease, chronic hypoxia, chronic arterial obstruction, or pulmonary hypertension with unclear or multifactorial mechanisms. In some embodiments, the composition is administered to the subject by inhalation. In other embodiments, the composition is administered to the subject by injection. In some embodiments, the composition further comprises at least one pharmaceutically acceptable carrier or excipient. In some embodiments, the composition further comprises at least one active ingredient. In preferred embodiments, the subject is a human subject.

Also disclosed herein is a method for treating acute kidney injury in a subject in need thereof, the method comprising administering a therapeutically effective amount of a composition comprising isolated mitochondria to the subject. In some embodiments, the isolated mitochondria are isolated porcine mitochondria. In some embodiments, the isolated mitochondria are isolated human mitochondria allogeneic to the subject. In some embodiments, the isolated mitochondria are isolated human mitochondria autologous to the subject. In some embodiments, administering the therapeutically effective amount of the composition reduces serum levels of one or more proinflammatory cytokines or proinflammatory mediators in the subject. In some embodiments, the one or more proinflammatory cytokines or proinflammatory mediators are selected from the group consisting of: monocyte chemoattractant protein 1 (MCP1), C3A, and C5a. In some embodiments, administering the therapeutically effective amount of the composition reduces kidney injury molecule-1 (KIM1) serum levels in the subject. In some embodiments, administering the therapeutically effective amount of the composition reduces blood urea nitrogen (BUN) levels in the subject. In some embodiments, administering the therapeutically effective amount of the composition reduces kidney weight in the subject.

Also disclosed herein is a method for treating a subject in cardiac arrest or undergoing resuscitation, the method comprising administering an effective amount of a composition comprising isolated mitochondria to the subject to facilitate transport thereof to a medical facility or medical treatment. In some embodiments, the isolated mitochondria are isolated porcine mitochondria. In some embodiments, the isolated mitochondria are isolated human mitochondria allogeneic to the subject. In some embodiments, the composition is administered to the subject by inhalation. In other embodiments, the composition is administered to the subject by injection. In some embodiments, the composition further comprises at least one pharmaceutically acceptable carrier or excipient. In some embodiments, the composition further comprises at least one active ingredient. In preferred embodiments, the subject is a human subject.

Also disclosed herein is a method of reducing inflammation in a subject in need thereof, the method comprising: (i) delivering isolated mitochondria to hematopoietic lineage cells isolated from the subject, and (ii) administering the hematopoietic lineage cells treated with the isolated mitochondria to the subject. In some embodiments, the isolated mitochondria are isolated porcine mitochondria. In some embodiments, the isolated mitochondria are isolated human mitochondria allogeneic to the subject. In some embodiments, the isolated mitochondria are isolated human mitochondria autologous to the subject. In preferred embodiments, the hematopoietic lineage cells treated with the isolated mitochondria have at least 1%, or at least 2%, or at least 5%, or at least 10%, or at least 20%, or at least 50%, or at least 100% improvement in mitochondrial function in comparison to corresponding hematopoietic cells not treated with the isolated mitochondria. In preferred embodiments, the subject is a human subject.

In some embodiments, the method further comprises the step of introducing a transgene encoding at least one heterologous protein into the isolated hematopoietic lineage cells prior to the step of delivering the isolated mitochondria to the hematopoietic lineage cells. In other embodiments, the method further comprises the step of introducing a transgene encoding at least one heterologous protein into the isolated hematopoietic lineage cells after the step of delivering the isolated mitochondria to the hematopoietic lineage cells.

In some embodiments, the isolated hematopoietic lineage cells are myeloid cells, myeloid precursor cells, or combinations thereof. In some embodiments, the hematopoietic lineage cells are isolated from the peripheral blood of the subject. In some embodiments, the subject has been treated with a stem cell mobilizing agent prior to isolation of the hematopoietic lineage cells from the peripheral blood. In preferred embodiments, the stem cell mobilizing agent is granulocyte-colony stimulating factor (G-CSF). In other embodiments, the hematopoietic lineage cells are isolated from the bone marrow of the subject.

Techniques for isolating and enriching cell subsets from blood or organ tissue of a subject are known in the art and include techniques such as flow cytometry, density centrifugation, and magnetic isolation (see, e.g., Salvagno, C. and de Visser, K. E., Methods Mol Biol. 2016; 1458:125-35, which is incorporated by reference in its entirety).

Various assays for determining levels and activities of protein (e.g., recombinant protein) are available, such as amplification/expression methods, immunohistochemistry methods, FISH and shed antigen assays, southern blotting, western blotting, or PCR techniques. Moreover, the protein expression or amplification may be evaluated using in vivo diagnostic assays, e.g. by administering a molecule (such as an antibody) which binds the protein to be detected and is tagged with a detectable label (e.g., a radioactive isotope) and externally scanning the patient for localization of the label. Thus, methods of measuring levels of protein levels in cells are generally known in the art and may be used to assess protein levels and/or activities in connection with the methods and compositions provided herein as applicable. These assays can be used to determine the effect of modifications to a recombinant protein encoded by a transgene. For example, these assays can be used to determine if the modifications result in a transgene not capable of producing normal levels or fully functional gene products or to confirm a transgene comprising a mutation of all or part of the recombinant protein.

In some embodiments, the method further comprises the step of differentiating the isolated hematopoietic lineage cells ex vivo prior to the step of delivering the isolated mitochondria to the isolated hematopoietic lineage cells. In other embodiments, the method further comprises the step of differentiating the isolated hematopoietic lineage cells ex vivo after the step of delivering the isolated mitochondria to the isolated hematopoietic lineage cells. In some embodiments, the isolated hematopoietic lineage cells are differentiated ex vivo into macrophages with a M1 or M2 phenotype.

In preferred embodiments, the hematopoietic lineage cells treated with the isolated mitochondria have reduced expression of NF-κB in comparison to corresponding hematopoietic lineage cells not treated with the isolated mitochondria. In particularly preferred embodiments, the hematopoietic lineage cells treated with the isolated mitochondria have reduced secretion of pro-inflammatory cytokines and chemokines such as MIP-1β (CCL4), PDGF-BB, RANTES (CCL5), soluble ICAM-1 (sICAM-1), M-CSF (CSF-1), IL-1β, IL-6, IL-8 (CXCL8), GDF-15, TGF-β1, or any combination thereof in comparison to corresponding hematopoietic lineage cells not treated with the isolated mitochondria.

In some embodiments, the isolated hematopoietic lineage cells treated with the isolated mitochondria are administered to the subject by injection. In some embodiments, the isolated hematopoietic lineage cells treated with the isolated mitochondria are administered to the subject as part of a microcarrier. In some embodiments the microcarriers are coated in a matrix, preferably having an extracellular component. In some embodiments the microcarriers are positively charged.

Non-limiting examples of myeloid cells or myeloid precursor cells are monocytes, macrophages, neutrophils, hematopoietic stem cells, and myeloid progenitor cells.

### V. Methods of Preserving an Organ, Tissue, Limb, or Other Body Part

Disclosed herein is a method of preserving a tissue or organ for transportation and transplantation, the method comprising delivering isolated mitochondria to a tissue or organ intended for transportation and transplantation, wherein the tissue or organ is procured from a deceased donor. In some embodiments, the isolated mitochondria are isolated porcine mitochondria. In some embodiments, the isolated mitochondria are isolated human mitochondria allogeneic to the deceased donor. In some embodiments, the isolated mitochondria are isolated human mitochondria autologous to the deceased donor.

In some embodiments, the isolated mitochondria are delivered to the tissue or organ within 24 hours of after the death of the donor. In other embodiments, the isolated mitochondria are delivered to the tissue or organ within 12 hours after the death of the donor. In other embodiments, the isolated mitochondria are delivered to the tissue or organ within four hours after the death of the donor.

In some embodiments, the method further comprises the step of procuring the tissue or organ from the deceased donor by harvesting the tissue or organ from the deceased donor. In some embodiments, the isolated mitochondria are delivered to the tissue or organ prior to harvesting the tissue or organ from the deceased donor. In other embodiments, the isolated mitochondria are delivered to the tissue or organ after harvesting the tissue or organ from the deceased donor. In some embodiments, the isolated mitochondria are delivered to the tissue or organ by injection. In some embodiments, the tissue or organ is a heart, liver, lung, blood vessel, ureter, trachea, skin patch, or kidney. In preferred embodiments, the tissue or organ is a human tissue or organ.

In preferred embodiments, the tissue or organ is a lung. In some embodiments, the isolated mitochondria are delivered to the lung by through the airway, intravenously, or intra-arterially. In other embodiments, the isolated mitochondria are delivered to the lung during EVLP. In particularly preferred embodiments, the lung is a human lung. In other preferred embodiments, the tissue or organ is a kidney. In some embodiments the isolated mitochondria are delivered to the kidney intravenously or intra-arterially.

Also disclosed herein is a method of preserving a limb or other body part lost due to traumatic amputation, the method comprising delivering isolated mitochondria to the limb or other body part after the traumatic amputation of the limb or other body part. In some embodiments, the isolated mitochondria are isolated porcine mitochondria. In some embodiments, the isolated mitochondria are isolated human mitochondria allogeneic to the limb or other body part. In some embodiments, the isolated mitochondria are isolated human mitochondria autologous to the limb or other body part. In some embodiments, the isolated mitochondria are delivered to the amputated limb or other body part no later than 15 minutes, 30 minutes, 1 hour, 4 hours, 8 hours, 12 hours or 24 hours after the traumatic amputation. In some embodiments, the isolated mitochondria are delivered to the amputated limb or other body part by injection. In preferred embodiments, the limb or other body part is a human limb or other body part.

### VI. Methods of Improving Cellular Function and Cell Therapy

Disclosed herein is a method of improving the cellular function of isolated cells, the method comprising delivering isolated mitochondria to the isolated cells. In some embodiments, the isolated mitochondria are isolated porcine mitochondria. In some embodiments, the isolated mitochondria are isolated human mitochondria allogeneic to the isolated cells. In preferred embodiments, the cells treated with the isolated mitochondria have at least 1%, or at least 2%, or at least 5%, or at least 10%, or at least 20%, or at least 50%, or at least 100% improvement in mitochondrial function in comparison to corresponding cells not treated with the isolated mitochondria.

In preferred embodiments, the isolated cells are human cells. In particularly preferred embodiments, the isolated cells are epithelial cells (e.g., type I alveolar cells, type II alveolar cells, small and large airway epithelial cells), endothelial cells (e.g., human pulmonary artery endothelial cells (HPAEC)), fibroblasts, progenitor cells (e.g., endothelial progenitor cells and mesenchymal stem cells), smooth muscle cells (e.g., pulmonary artery smooth muscle cells), immune cells (e.g., hematopoietic lineage cells), mesenchymal cells, pericytes, and any combination thereof.

In some embodiments, the cells treated with the isolated mitochondria have increased extracellular vesicle secretion in comparison to corresponding cells not treated with the isolated mitochondria. In some embodiments, the cells treated with the isolated mitochondria have an altered extracellular vesicle composition in comparison to corresponding cells not treated with the isolated mitochondria. In preferred embodiments, the altered extracellular vesicle composition is altered in terms of protein content, nucleic acid content, lipid content, or any combination thereof.

In some embodiments, the method further comprises the step of introducing a transgene encoding at least one heterologous protein into the isolated cells prior to the step of delivering the isolated mitochondria to the isolated cells. In other embodiments, the method comprises the step of introducing a transgene encoding at least one heterologous protein into the isolated cells after the step of delivering the isolated mitochondria to the isolated cells. In preferred embodiments, the heterologous protein is secreted from the cells in extracellular vesicles.

In some embodiments, the cells treated with the isolated mitochondria have reduced cellular apoptosis, increased cell viability, reduced autophagy, reduced mitophagy, reduced senescence, reduced mitochondrial stress signaling, reduced cell damage, reduced cellular inflammation, reduced reactive oxygen species production, increased cellular barrier function, increased angiogenesis, increased cellular adhesion, increased growth kinetics, or any combination thereof in comparison to corresponding cells not treated with the isolated mitochondria. In preferred embodiments, the reduced cell damage is associated with reduced TLR9 expression, altered HO-1 expression, reduced cytosolic mtDNA, or any combination thereof. In some embodiments, the altered HO-1 expression is increased HO-1 expression after cold exposure. In preferred embodiments, the reduced cellular apoptosis, increased cell viability, reduced mitochondrial stress signaling, and/or reduced cell damage is associated with reduced NF-κB, MAPK14, JNK, p53 expression, or any combination thereof. In preferred embodiments, the reduced cellular apoptosis is associated with reduced pro-apoptotic marker expression. In particularly preferred embodiments, the reduced cellular apoptosis is associated with reduced expression of pro-apoptotic initiators (BIM, PUMA), pro-apoptotic effectors (BAX, BAK), apoptogenic factors (SMAC, DIABLO, BID, BAD, etc.), or any combination thereof. In preferred embodiments, the reduced cellular apoptosis is associated with increased anti-apoptotic marker expression. In particularly preferred embodiments, the reduced cellular apoptosis is associated with increased expression of BCL-2, BCL-XL, BCL-W, A1/BFL-1, MCL-1, or any combination thereof.

In some embodiments, the cells treated with the isolated mitochondria have increased glucose uptake and decreased lactate production in comparison to corresponding cells not treated with the isolated mitochondria. In preferred embodiments, the increased glucose uptake and decreased lactate production is associated with increased expression of HK, VDAC1, GLUT, AKT1, or any combination thereof.

In preferred embodiments, the cells treated with the isolated mitochondria have improved cellular adhesion and growth kinetics on a two-dimensional or three-dimensional cell support in comparison to corresponding cells not treated with the isolated mitochondria. In some embodiments, the two-dimensional or three-dimensional cell support is a microcarrier. In some embodiments, the two-dimensional or three-dimensional cell support comprises one or more extracellular matrix components.

In preferred embodiments, the cells treated with the isolated mitochondria maintain viability in cold ischemia or cold storage longer than corresponding cells not treated with the isolated mitochondria.

Non-limiting examples of isolated cells epithelial cells (e.g., type I alveolar cells, type II alveolar cells, small and large airway epithelial cells), endothelial cells (e.g., human pulmonary artery endothelial cells (HPAEC)), fibroblasts, progenitor cells (e.g., endothelial progenitor cells and mesenchymal stem cells), smooth muscle cells (e.g., pulmonary artery smooth muscle cells), skeletal muscle cells, cardiomyocytes, hepatocytes, immune cells (e.g., hematopoietic lineage cells), mesenchymal cells, pericytes, neuronal cells, and any combination thereof.

Also disclosed herein is a method of improving cell therapy in a subject in need thereof, the method comprising: (i) delivering isolated mitochondria to isolated cells in vitro, and (ii) administering the cells treated with the isolated mitochondria to the subject. In some embodiments, the isolated mitochondria are isolated porcine mitochondria. In some embodiments, the isolated mitochondria are isolated human mitochondria allogeneic to the subject. In some embodiments, the isolated mitochondria are isolated human mitochondria autologous to the subject. In some embodiments, the method further comprises the step of isolating the autologous cells from the subject prior to the step of delivering isolated mitochondria to the isolated cells in vitro. In preferred embodiments, the cells treated with the isolated mitochondria have at least 1%, or at least 2%, or at least 5%, or at least 10%, or at least 20%, or at least 50%, or at least 100% improvement in mitochondrial function in comparison to corresponding cells not treated with the isolated mitochondria. In preferred embodiments, the subject is a human subject.

In some embodiments, the isolated cells are allogeneic cells. In other embodiments, the isolated cells are autologous cells. In preferred embodiments, the isolated cells are human cells. In particularly preferred embodiments, the isolated cells are epithelial cells (e.g., type I alveolar cells, type II alveolar cells, small and large airway epithelial cells), endothelial cells (e.g., human pulmonary artery endothelial cells (HPAEC)), fibroblasts, progenitor cells (e.g., endothelial progenitor cells and mesenchymal stem cells), smooth muscle cells (e.g., pulmonary artery smooth muscle cells), skeletal muscle cells, cardiomyocytes, hepatocytes, immune cells (e.g., hematopoietic lineage cells), mesenchymal cells, pericytes, neuronal cells, or any combination thereof.

In some embodiments, the cells treated with the isolated mitochondria have increased extracellular vesicle secretion in comparison to corresponding cells not treated with the isolated mitochondria. In some embodiments, the cells treated with the isolated mitochondria have an altered extracellular vesicle composition in comparison to corresponding cells not treated with the isolated mitochondria. In preferred embodiments, the altered extracellular vesicle composition is altered in terms of protein content, nucleic acid content, lipid content, or any combination thereof.

In some embodiments, the method further comprises the step of introducing a transgene encoding at least one heterologous protein into the isolated cells prior to the step of delivering the isolated mitochondria to the isolated cells. In other embodiments, the method further comprises the step of introducing a transgene encoding at least one heterologous protein into the isolated cells after the step of delivering the isolated mitochondria to the isolated cells. In preferred embodiments, the heterologous protein is secreted from the cells in extracellular vesicles.

In some embodiments, the cells treated with the isolated mitochondria are administered to the subject by injection. In other embodiments, the cells treated with the isolated mitochondria are administered to the subject through the airway. In some embodiments, cells treated with the isolated mitochondria are administered to the subject as part of a microcarrier.

In some embodiments, the treated cells have reduced cellular apoptosis, increased cell viability, reduced autophagy, reduced mitophagy, reduced senescence, reduced mitochondrial stress signaling, reduced reactive oxygen species production, reduced cell damage, reduced cellular inflammation, increased cellular barrier function, increased angiogenesis, increased cellular adhesion, increased growth kinetics, or any combination thereof in comparison to corresponding cells not treated with the isolated mitochondria. In preferred embodiments, the reduced cell damage is associated with reduced TLR9 expression, altered HO-1 expression, reduced cytosolic mtDNA, or any combination thereof. In some embodiments, the altered HO-1 expression is increased HO-1 expression after cold exposure. In preferred embodiments, the reduced cellular apoptosis, increased cell viability, reduced mitochondrial stress signaling, and/or reduced cell damage is associated with reduced NF-κB, MAPK14, JNK, p53 expression, or any combination thereof. In preferred embodiments, the reduced cellular apoptosis is associated with reduced pro-apoptotic marker expression. In particularly preferred embodiments, the reduced cellular apoptosis is associated with reduced expression of pro-apoptotic initiators (BIM, PUMA), pro-apoptotic effectors (BAX, BAK), apoptogenic factors (SMAC, DIABLO, BID, BAD, etc.), or any combination thereof. In preferred embodiments, the reduced cellular apoptosis is associated with increased anti-apoptotic marker expression. In particularly preferred embodiments, the reduced cellular apoptosis is associated with increased expression of BCL-2, BCL-XL, BCL-W, A1/BFL-1, MCL-1, or any combination thereof.

In some embodiments, the treated cells have increased glucose uptake and decreased lactate production in comparison to corresponding cells not treated with the isolated mitochondria. In preferred embodiments, the increased glucose uptake and decreased lactate production is associated with increased expression of HK, VDAC1, GLUT, AKT1, or any combination thereof, by at least 1%, or at least 2%, or at least 5%, or at least 10%, or at least 20%, or at least 50%, or at least 80%.

In preferred embodiments, the cells treated with the isolated mitochondria have improved cellular adhesion and growth kinetics on a two-dimensional or three-dimensional cell support in comparison to corresponding cells not treated with the isolated mitochondria. In some embodiments, the two-dimensional or three-dimensional cell support is a microcarrier. In some embodiments, the two-dimensional or three-dimensional cell support comprises one or more extracellular matrix components.

In preferred embodiments, the cells treated with the isolated mitochondria maintain viability in cold ischemia longer than corresponding cells not treated with the isolated mitochondria.

### VII. Methods for Improving Cold Transportation, Shipment, and Storage of Isolated Cells

Also disclosed herein is a method for improving the cold transportation, cold shipment, or cold storage of isolated cells, the method comprising delivering isolated mitochondria to the isolated cells before, during or after cold transportation, cold shipment, or cold storage, wherein the cells treated with the isolated mitochondria have at least 1%, or at least 2%, or at least 5%, or at least 10%, or at least 20%, or at least 50%, or at least 100% improvement in viability in comparison to cells of corresponding cells not treated with the isolated mitochondria. In some embodiments, the isolated mitochondria are isolated porcine mitochondria. In some embodiments, the isolated mitochondria are isolated human mitochondria allogeneic to the cells. In some embodiments, the isolated mitochondria are isolated human mitochondria autologous to the cells. In preferred embodiments, the cells treated with the isolated mitochondria have at least 1%, or at least 2%, or at least 5%, or at least 10%, or at least 20%, or at least 50%, or at least 100% improvement in mitochondrial function in comparison to cells of corresponding cells not treated with the isolated mitochondria. In preferred embodiments, the isolated cells are human cells. In particularly preferred embodiments, the isolated cells are epithelial cells (e.g., type I alveolar cells, type II alveolar cells, small and large airway epithelial cells), endothelial cells (e.g., human pulmonary artery endothelial cells (HPAEC)), fibroblasts, progenitor cells (e.g., endothelial progenitor cells and mesenchymal stem cells), smooth muscle cells (e.g., pulmonary artery smooth muscle cells), immune cells (e.g., hematopoietic lineage cells), mesenchymal cells, or pericytes.

In preferred embodiments, the cells treated with the isolated mitochondria have reduced production of ROS-mediated oxidative byproducts, improved cell viability, reduced necrosis, reduced cell lysis, increased total levels of cellular ATP, reduced inflammatory cytokine secretion, or any combination thereof in comparison to corresponding cells not treated with the isolated mitochondria. In some embodiments, the inflammatory cytokines comprise IL-6, IL-8, and IFN-γ. In some embodiments, the ROS-mediated oxidative byproducts comprise 4-HNE and 8-OHdG.

In some embodiments, the method further comprises the step of cryopreserving the human cells treated with the isolated mitochondria. In preferred embodiments, the human cells treated with the isolated mitochondria are cryopreserved by step-down liquid N₂ freezing. In some embodiments, the cells treated with the isolated mitochondria are maintained in a solution comprising a lipid, a protein, a saccharide, an oligosaccharide a polysaccharide, or any combination thereof. In preferred embodiments, the cells treated with the isolated mitochondria are maintained in a solution comprising trehalose, sucrose, glycerol, PlasmaLyte, CryoStor, dimethyl sulfoxide, lipid, glutamate, PEGs, PVAs, albumin, or any combination thereof. In particularly preferred embodiments, the isolated mitochondria are present in the solution. The isolated mitochondria may be delivered to the human cells prior to the step of cryopreserving the human cells, during the step of cryopreserving the human cells, upon thawing from cryopreservation, or any combination thereof.

In some embodiments, the isolated cells are allogeneic cells. In other embodiments, the isolated cells are autologous cells. In preferred embodiments, the isolated cells are human cells. In particularly preferred embodiments, the isolated cells are epithelial cells (e.g., type I alveolar cells, type II alveolar cells, small and large airway epithelial cells), endothelial cells (e.g., human pulmonary artery endothelial cells (HPAEC)), fibroblasts, progenitor cells (e.g., endothelial progenitor cells and mesenchymal stem cells), smooth muscle cells (e.g., pulmonary artery smooth muscle cells), skeletal muscle cells, cardiomyocytes, hepatocytes, immune cells (e.g., hematopoietic lineage cells), mesenchymal cells, pericytes, neuronal cells, or any combination thereof.

### VIII. Methods for Preservation of Isolated Mitochondria

Disclosed herein is a method for cryopreservation of isolated mitochondria, such as porcine mitochondria, comprising freezing isolated mitochondria in a freezing buffer comprising a cryprotectant. In some embodiments, the isolated mitochondria are isolated porcine mitochondria. In some embodiments, the isolated mitochondria are isolated human mitochondria. In some embodiments, the method further comprises isolating the mitochondria from cells or tissue. In some embodiments, the cryoprotectant is a lipid, a protein, a saccharide, a disaccharide, an oligosaccharide a polysaccharide, or any combination thereof. In some embodiments, the isolated mitochondria are stored at physiologic pH using an isotonic buffer and may optionally include a polypeptide, protein, or other agent to preserve mitochondria membrane integrity. For example, the cold storage buffer can have a pH between 7.0 and 7.5, such as about 7.2, 7.35, or 7.4. In preferred embodiments, the cryoprotectant is trehalose, sucrose, glycerol, PlasmaLyte, CryoStor, DMSO, glutamate, PEGs, PVAs, albumin, or any combination thereof. In some embodiments, the isolated mitochondria are cryopreserved by step-down liquid N₂ freezing. In some embodiments, the trehalose or other cryoprotectant can be present in amounts from 100-500 mM, 200-400 mM, 250-350 mM, or 275-325 mM. The mitochondria can be held at a temperature of −20° C. or below, −40° C. or below, −60° C. or below, −70° C. or below, or −80° C. or below.

In some embodiments, the method further comprises thawing the frozen isolated mitochondria and assessing the health and/or function of the thawed isolated mitochondria by measuring one or more of: mitochondrial swelling, mitochondria membrane transition pore (mPTP) opening, mitochondrial respiration, mitochondria membrane potential, complete mitochondria permeability, and mitochondrial swelling. In some preferred embodiments, the mitochondria are porcine mitochondria. In other embodiments, the method further comprises thawing the frozen isolated mitochondria and assessing the health and/or function of the thawed isolated mitochondria by scoring gross mitochondria morphology and/or measuring average mitochondria size. In some embodiments, the thawed isolated mitochondria can be sorted based on pre-defined criteria using techniques such as flow cytometry, such as to isolate only healthy and/or functional mitochondria.

Also disclosed herein is a method for long-term storage of isolated mitochondria, such as porcine mitochondria, the method comprising: (i) isolating mitochondria from cells or tissue, (ii) suspending the isolated mitochondria in a cold storage buffer, (iii) freezing the isolated mitochondria in the cold storage buffer at a temperature from about −70° C. to about −100° C., and (iv) maintaining the frozen isolated mitochondria at a temperature from about −70° C. to about −100° C. for 24 hours or longer. In some embodiments, the isolated mitochondria are isolated porcine mitochondria. In some embodiments, the isolated mitochondria are isolated human mitochondria.

In some embodiments, the method comprises freezing isolated mitochondria in the cold storage buffer at a temperature from about −70° C. to about −100° C., and maintaining the frozen isolated mitochondria at a temperature from about −70° C. to about −100° C. for 24 hours or longer. In preferred embodiments, the isolated mitochondria in the cold storage buffer are frozen at a temperature from about −75° C. to about −95° C., and wherein the frozen isolated mitochondria are maintained at a temperature from about −75° C. to about −95° C. In particularly preferred embodiments, the isolated mitochondria in the cold storage buffer are frozen at a temperature from about −80° C. to about −90° C., and wherein the frozen isolated mitochondria are maintained at a temperature from about −80° C. to about −90° C. In some embodiments, the cold storage buffer comprises trehalose, sucrose, glycerol, CryoStor, or any combination thereof. In preferred embodiments, the cold storage buffer is isotonic and has a pH of about 7.0 to about 7.5. In particularly preferred embodiments, the cold storage buffer is isotonic and has a pH of about 7.2. In particularly preferred embodiments, the cold storage buffer comprises trehalose. In particularly preferred embodiments, the cold storage buffer comprises 300 mM trehalose, 10 mM HEPES, 10 mM KCl, 1 mM EGTA, 0.1% fatty acid-free BSA. In some embodiments, the frozen isolated mitochondria are maintained at the temperature for 1 week or longer. In some embodiments, the frozen isolated mitochondria are maintained at the temperature for 1 month, 2 months, 3 months, 4 months, 5 months, 6 months, 7 months, 8 months, 9 months, 10 months, 11 months, 1 year, or longer. In some embodiments, the method further comprises: (v) thawing the frozen isolated mitochondria, and (vi) assessing the health and/or function of the thawed isolated mitochondria by measuring one or more of: mitochondrial swelling, mitochondria membrane transition pore (mPTP) opening, mitochondrial respiration, mitochondria membrane potential, complete mitochondria permeability, and mitochondrial swelling. In some embodiments, the method further comprises: (v) thawing the frozen isolated mitochondria, (vi) assessing the health of the thawed isolated mitochondria by measuring mitochondrial swelling using flow cytometry, and (vii) isolating healthy mitochondria from mitochondria having a swelling phenotype using flow-cytometry-assisted cell sorting. In other embodiments, the method further comprises: (v) thawing the frozen isolated mitochondria, and (vi) assessing the health of the thawed isolated mitochondria by scoring gross mitochondria morphology and/or measuring average mitochondria size. In some preferred embodiments, the mitochondria are porcine mitochondria.

### IX. Methods for Detecting Porcine Mitochondria in Human Cells

Disclosed herein is a method for detecting porcine mitochondria in a human cell, tissue, or organ sample, the method comprising detecting in vitro or ex vivo the presence of a nucleic acid marker in the human cell, tissue, or organ sample, wherein the nucleic acid marker comprises a sequence of mitochondrial DNA or RNA, and wherein the nucleic acid marker is present in porcine mitochondria and absent in human mitochondria. In preferred embodiments, the method further comprises quantitating the amount of the nucleic acid marker in the human cell, tissue, or organ sample.

In some embodiments, the method further comprises the step of amplifying the nucleic acid marker by polymerase chain reaction (PCR). In some embodiments, the presence of the nucleic acid marker is detected by PCR using a primer pair, wherein at least one of the primers of the primer pair specifically hybridizes to the nucleic acid marker. In other embodiments, the presence of the nucleic acid marker is detected using a nucleic acid probe that specifically hybridizes to the nucleic acid marker.

### X. Compositions Comprising Human Cells with Exogenous Mitochondria

Disclosed herein is a composition comprising human cells, wherein the cytosol of the human cells comprises exogenous mitochondria, wherein the human cells of the composition have at least 1%, or at least 2%, or at least 5%, or at least 10%, or at least 20%, or at least 50%, or at least 100% improvement in mitochondrial function in comparison to corresponding human cells lacking exogenous mitochondria, and wherein the improved mitochondrial function is increased oxygen consumption and/or increased ATP synthesis, by at least 1%, or at least 2%, or at least 5%, or at least 10%, or at least 20%, or at least 50%, or at least 100%. In some embodiments, the exogenous mitochondria are porcine mitochondria. In some embodiments, the exogenous mitochondria are human mitochondria allogeneic to the human cells. In some embodiments, the exogenous mitochondria are derived from a porcine heart. In some embodiments, the human cells are epithelial cells (e.g., type I alveolar cells, type II alveolar cells, small and large airway epithelial cells), endothelial cells (e.g., human pulmonary artery endothelial cells (HPAEC)), fibroblasts, progenitor cells (e.g., endothelial progenitor cells and mesenchymal stem cells), smooth muscle cells (e.g., pulmonary artery smooth muscle cells), skeletal muscle cells, cardiomyocytes, hepatocytes, immune cells (e.g., hematopoietic lineage cells), mesenchymal cells, pericytes, neuronal cells, or any combination thereof.

In some embodiments, the human cells have increased extracellular vesicle secretion in comparison to corresponding human cells lacking exogenous mitochondria. In some embodiments, the human cells have an altered extracellular vesicle composition in comparison to corresponding human cells lacking exogenous mitochondria. In preferred embodiments, the altered extracellular vesicle composition is altered in terms of protein content, nucleic acid content, lipid content, or any combination thereof.

In some embodiments, the human cells further comprise a transgene encoding at least one heterologous protein. In some embodiments, transcription of the transgene occurs in the nucleus of the human cell. In some embodiments, the transgene is stably integrated in the nuclear DNA of the human cell. In preferred embodiments, the heterologous protein is secreted from the human cells in extracellular vesicles. In other embodiments, transcription of the transgene occurs in the exogenous mitochondria. In some embodiments, the transgene is stably integrated in the mitochondrial DNA (mtDNA) of the exogenous mitochondria.

In preferred embodiments, the human cells maintain viability in cold ischemia or cold storage longer than corresponding human cells lacking exogenous mitochondria.

In preferred embodiments, the human cells have reduced cellular apoptosis, increased cell viability, reduced autophagy, reduced mitophagy, reduced senescence, reduced mitochondrial stress signaling, reduced reactive oxygen species production, reduced cellular inflammation, reduced cell damage, increased cellular adhesion, increased cellular barrier function, increased angiogenesis, increased growth kinetics, or any combination thereof in comparison to corresponding human cells lacking exogenous mitochondria. In particularly preferred embodiments, the reduced cell damage is associated with reduced TLR9 expression, altered HO-1 expression, reduced cytosolic mtDNA, or any combination thereof. In some embodiments, the altered HO-1 expression is increased HO-1 expression after cold exposure. In preferred embodiments, the reduced cellular apoptosis, increased cell viability, reduced mitochondrial stress signaling, and/or reduced cell damage is associated with reduced NF-κB, MAPK14, JNK, p53 expression. In preferred embodiments, the reduced cellular apoptosis is associated with reduced pro-apoptotic marker expression. In particularly preferred embodiments, the reduced cellular apoptosis is associated with reduced expression of pro-apoptotic initiators (BIM, PUMA), pro-apoptotic effectors (BAX, BAK), apoptogenic factors (SMAC, DIABLO, BID, BAD, etc.), or any combination thereof. In preferred embodiments, the reduced cellular apoptosis is associated with increased anti-apoptotic marker expression. In particularly preferred embodiments, the reduced cellular apoptosis is associated with increased expression of BCL-2, BCL-XL, BCL-W, A1/BFL-1, MCL-1, or any combination thereof.

In some embodiments, the human cells have increased glucose uptake and decreased lactate production in comparison to corresponding human cells not treated with exogenous mitochondria. In preferred embodiments, the increased glucose uptake and decreased lactate production is associated with increased expression of HK, VDAC1, GLUT, AKT1, or any combination thereof.

In preferred embodiments, the human cells have improved cellular adhesion and growth kinetics on a two-dimensional or three-dimensional cell support in comparison to corresponding human cells lacking exogenous mitochondria. In some embodiments, the composition further comprises a two-dimensional or three-dimensional cell support. In some embodiments, the two-dimensional or three-dimensional cell support is a microcarrier. In some embodiments, the two-dimensional or three-dimensional cell support comprises one or more extracellular matrix components.

In some embodiments, the composition further comprises at least one pharmaceutically acceptable carrier or excipient. In some embodiments, the composition further comprises at least one active ingredient.

The isolated polypeptides and recombinant proteins described herein can be produced by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g. Mark, D. F., et al., Proc Natl Acad Sci USA. 1984 September; 81(18):5662-6 and U.S. Pat. No. 4,588,585 (incorporated herein by reference in their entireties).

A DNA sequence (e.g., a transgene) encoding one or more polypeptides (e.g., recombinant proteins) of interest can be constructed by chemical synthesis using an oligonucleotide synthesizer. Such oligonucleotides can be designed based on the amino acid sequence of the desired polypeptide and selecting those codons that are favored in the host cell in which the polypeptide of interest will be produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis or another method), the polynucleotide sequences encoding a particular isolated polypeptide of interest will be inserted into an expression vector and operatively linked to an expression control sequence appropriate for expression of the protein in a desired host. Proper assembly can be confirmed by nucleotide sequencing, restriction mapping, and expression of a biologically active polypeptide in a suitable host. As is known in the art, in order to obtain high expression levels of a transfected gene in a host, the gene can be operatively linked to transcriptional and translational expression control sequences that are functional in the chosen expression host.

In certain embodiments, recombinant expression vectors are used to amplify and express DNA (e.g., a transgene) encoding one or more polypeptides (e.g., recombinant proteins) of interest. Recombinant expression vectors are replicable DNA constructs which have synthetic or cDNA-derived DNA fragments encoding a polypeptide of interest, operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail below. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Structural elements intended for use in yeast expression systems include a leader sequence allowing extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

The choice of expression control sequence and expression vector will depend upon the choice of host. A wide variety of expression host/vector combinations can be employed. Useful expression vectors for eukaryotic hosts, include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR1, pBR322, pMB9 and their derivatives, wider host range plasmids, such as M13 and filamentous single-stranded DNA phages.

Suitable host cells for expression one or more polypeptides of interest include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin as described below. Cell-free translation systems could also be employed. Appropriate cloning and expression vectors for use with bacterial, fungal, yeast, and mammalian cellular hosts are described by Pouwels et al. (Cloning Vectors: A Laboratory Manual, Elsevier, N.Y., 1985), the relevant disclosure of which is hereby incorporated by reference. Additional information regarding methods of protein production can be found, e.g., in U.S. Patent Publication No. 2008/0187954, U.S. Pat. Nos. 6,413,746 and 6,660,501, and International Patent Publication No. WO 04009823, each of which is hereby incorporated by reference herein in its entirety.

The proteins produced by a transformed host can be purified according to any suitable method. Such standard methods include chromatography (e.g., ion exchange, affinity and sizing column chromatography), gradient, centrifugation, differential solubility, or by any other standard technique for protein purification. Affinity tags such as hexahistidine, maltose binding domain, influenza coat sequence and glutathione-S-transferase can be attached to the protein to allow purification by passage over an appropriate affinity column. Isolated proteins can also be physically characterized using such techniques as proteolysis, nuclear magnetic resonance and x-ray crystallography.

In certain embodiments of the invention, cells harboring at least one integrative or non-integrative vector may be identified in vitro by including a reporter gene in the expression vector. Generally, a selectable reporter is one that confers a property that allows for selection. A positive selectable reporter is one in which the presence of the reporter gene allows for its selection, while a negative selectable reporter is one in which its presence prevents its selection. An example of a positive selectable marker is a drug resistance marker (genes that confer resistance to neomycin, puromycin, hygromycin, DHFR, GPT, zeocin and histidinol). Other types of reporters include screenable reporters such as GFP.

Various assays for determining levels and activities of protein (e.g., recombinant protein) are available, such as amplification/expression methods, immunohistochemistry methods, FISH and shed antigen assays, southern blotting, western blotting, or PCR techniques. Moreover, the protein expression or amplification may be evaluated using in vivo diagnostic assays, e.g. by administering a molecule (such as an antibody) which binds the protein to be detected and is tagged with a detectable label (e.g., a radioactive isotope) and externally scanning the patient for localization of the label. Thus, methods of measuring levels of protein levels in cells are generally known in the art and may be used to assess protein levels and/or activities in connection with the methods and compositions provided herein as applicable. These assays can be used to determine the effect of modifications to a recombinant protein encoded by a transgene. For example, these assays can be used to determine if the modifications result in a transgene not capable of producing normal levels or fully functional gene products or to confirm a transgene comprising a mutation of all or part of the recombinant protein.

Upon formulation, aqueous solutions for parenteral administration will be administered in a manner compatible with the dosage formulation and in such amount as is therapeutically or prophylactically effective. The solution may be suitably buffered if necessary and the liquid diluent first rendered isotonic with sufficient saline or glucose. These particular aqueous solutions are especially suitable for intravascular administration. In this connection, sterile aqueous media, which can be employed will be known to those of skill in the art in light of the present disclosure.

The appropriate dosage of the cells, mitochondria, or additional active agents of the compositions described herein depends on: the type of disease, pathological condition, or disorder to be treated; the severity and course of the disease, pathological condition, or disorder; the responsiveness of the disease, pathological condition, or disorder to previous therapy; the subject's clinical history; and so on. The composition can be administered one time or over a series of treatments lasting from several days to several months, or until a cure is effected or a diminution of the state of the disease, pathological condition, or disorder is achieved.

The stem cells according to certain aspects of the present invention may be cultured and maintained in an essentially undifferentiated state using defined, feeder-independent culture system, such as a TeSR medium (Ludwig et al., Nat. Biotechnol. 2006, 24(2):185-7 and Ludwig et al., Nat. Methods 2006, 3(8):637-46). Feeder-independent culture systems and media may be used to culture stem cells. These approaches allow stem cells to grow in an essentially undifferentiated state without the need for mouse fibroblast “feeder layers.”

The cell culture medium for culturing cells according to certain aspects of the present invention can be prepared using a medium used for culturing animal cells as its basal medium, such as any of TeSR, BME, BGJb, CMRL 1066, Glasgow MEM, Improved MEM Zinc Option, IMDM, Medium 199, Eagle MEM, aMEM, DMEM, Ham, RPMI 1640, and Fischer's media, as well as any combinations thereof, but the medium is not particularly limited thereto as far as it can be used for culturing animal cells. Particularly, the medium may be xeno-free or chemically defined.

The cell culture medium can be a serum-containing or serum-free medium. The serum-free medium refers to media with no unprocessed or unpurified serum, and accordingly can include media with purified blood-derived components or animal tissue-derived components (such as growth factors). From the aspect of preventing contamination with heterogeneous animal-derived components, serum can be derived from the same animal as that of the stem cell(s).

The cell culture medium may contain or may not contain any alternatives to serum. The alternatives to serum can include materials which appropriately contain albumin (such as lipid-rich albumin, albumin substitutes such as recombinant albumin, plant starch, dextrans and protein hydrolysates), transferrin (or other iron transporters), fatty acids, insulin, collagen precursors, trace elements, 2-mercaptoethanol, 3′-thiolglycerol, human plasma lysate, or equivalents thereto. The alternatives to serum can be prepared by the method disclosed in International Publication No. 98/30679, for example. Alternatively, any commercially available materials can be used for more convenience. The commercially available materials include knockout Serum Replacement (KSR), Chemically-defined Lipid concentrated (Gibco), and Glutamax (Gibco).

The cell culture medium can also contain fatty acids or lipids, glucose, amino acids (such as non-essential amino acids), vitamin(s), growth factors, cytokines, antioxidant substances, 2-mercaptoethanol, pyruvic acid, buffering agents, and inorganic salts. The concentration of 2-mercaptoethanol can be, for example, about 0.05 to 1.0 mM, and particularly about 0.1 to 0.5 mM, but the concentration is particularly not limited thereto as long as it is appropriate for culturing the stem cell(s).

A culture vessel used for culturing the cells according to certain aspects of the present invention can include, but is particularly not limited to: flask, flask for tissue culture, dish, petri dish, dish for tissue culture, multi dish, micro plate, micro-well plate, multi plate, multi-well plate, micro slide, chamber slide, tube, tray, CellSTACK®, chambers, culture bag, and roller bottle, as long as it is capable of culturing the stem cells therein. The cells may be cultured in a volume of at least or about 0.2, 0.5, 1, 2, 5, 10, 20, 30, 40, 50 ml, 100 ml, 150 ml, 200 ml, 250 ml, 300 ml, 350 ml, 400 ml, 450 ml, 500 ml, 550 ml, 600 ml, 800 ml, 1000 ml, 1500 ml, or any range derivable therein, depending on the needs of the culture. In a certain embodiment, the culture vessel may be a bioreactor, which may refer to any device or system that supports a biologically active environment. The bioreactor may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

The culture vessel can be cellular adhesive or non-adhesive and selected depending on the purpose. The cellular adhesive culture vessel can be coated with any of substrates for cell adhesion such as extracellular matrix (ECM) to improve the adhesiveness of the vessel surface to the cells. The substrate for cell adhesion can be any material intended to attach cells. The substrate for cell adhesion includes collagen, gelatin, poly-L-lysine, poly-D-lysine, laminin, and fibronectin, fragments or mixtures thereof.

The cells according to certain aspects of the present invention may also be cultured by suspension culture, including suspension culture on carriers (Fernandes et al., Nature Cell Biology, 2004; 6:1082-93) or gel/biopolymer encapsulation (United States Publication 2007/0116680). The term suspension culture of the cells means that the cells are cultured under non-adherent condition with respect to the culture vessel or feeder cells (if used) in a medium.

Various approaches described herein may be used with the present invention to differentiate stem cells into cells or cell lineages including, but not limited to, keratinocytes, hematopoietic cells, myocytes, fibroblasts, epithelia cells, and epidermal cells, and tissues or organs derived therefrom.

### EXAMPLES

It is understood that the examples and embodiments disclosed herein are for illustrative purposes only and that various modifications or changes in light thereof will be suggested to persons skilled in the art and are to be included within the spirit and purview of this application.

### Example 1

### Treatment of Cells with Porcine Mitochondria Improves Oxygen Consumption Rate after Acute and Chronic Cold Exposure

To isolate porcine mitochondria, the entire left ventricle was removed from a freshly excised pig heart and placed in ice cold washing media (300 mM sucrose, 1 mM EGTA, 10 mM HEPES (pH 7.4)) for transport. A one inch square piece of tissue was cut from the left ventricle and transferred to a pre-chilled 50 ml conical tube containing 20 mL ice cold Trehalose buffer. The tissue sample was minced to obtain sample pieces of approximately 1-2 mm in size. The sample was enzymatically digested in a subtilisin A solution (5 mg/ml subtilisin A in 250 μl of Trehalose buffer) on ice for 10 minutes and homogenized using a Potter-Elvehjem pattern tissue homogenizer (3-7 passes). The sample was then passed through gauze into a 50 ml conical tube. The sample was centrifuged (10 minutes at 4° C. at 500 g) and the supernatant was decanted into a fresh 50 mL conical tube. The sample was centrifuged for 10 minutes at 4° C. at 15,000 g. The supernatant was discarded. The sample pellet was resuspended in 500 μl of Trehalose buffer and transferred to a 1.5 ml Eppendorf tube. The 50 ml conical tube was then rinsed with 500 μl of Trehalose buffer, which was added to the sample in the 1.5 ml Eppendorf tube. The sample pellet was washed three times by centrifugation (10 minutes at 4° C. at 15,000 g) and resuspended in 1 mL Trehalose buffer.

It has previously been shown that the oxygen consumption rate (OCR), which is an indicator of mitochondrial respiration, can be determined in real-time in live cells using a Seahorse assay using a Seahorse Extracellular Flux (XF) Analyzer (Seahorse Bioscience, Inc., North Billerica, Mass.). See Rose, S., et al., PLOS One (2014), 9(1):e85436 (“Rose et al.”), which is incorporated by reference herein in its entirety. Rose et al. showed that multiple measures of mitochondrial respiration, such as basal respiration, ATP-linked respiration, proton leak respiration, and reserve capacity, can be derived by treating cells with specific inhibitors. Id. In particular, cells can be treated with oligomycin, which is an inhibitor of complex V, to derive ATP-linked respiration and proton leak respiration. Carbonyl cyanide-p-trifluoromethoxyphenyl-hydrazon (FCCP), which is a protonophore, collapses the mitochondria inner membrane gradient and causes the electron transport chain (ETC) to function at its maximal rate. Id. Therefore, maximal respiratory capacity can be determined by treating cells with FCCP. Id. Non-mitochondrial respiration can be determined by treatment with a combination of rotenone, which is a complex I inhibitor, and antimycin A, which is a complex III inhibitor, to effectively shut down ETC function.

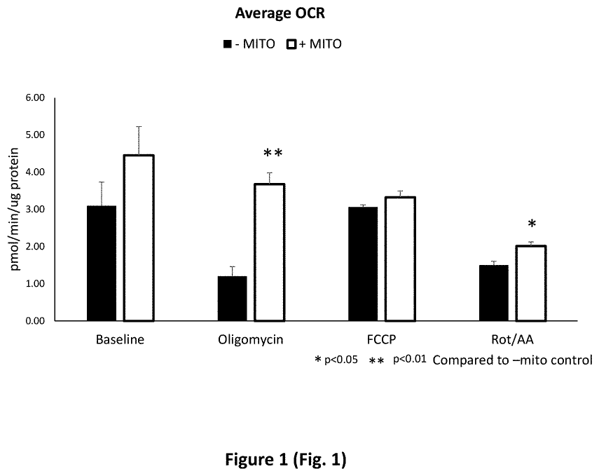

The effects of treatment of human pulmonary artery endothelial cells (HPAEC) with the isolated porcine mitochondria on oxygen consumption rate (OCR) after acute cold exposure were determined by Seahorse assay. HPAEC were placed in 4° C. for 6 hours. HPAEC recovered in normoxia for 1 hour at 37° C. in the presence of either 20 uL of mitochondria suspension (respiration buffer containing 29 particles per cell; “+ MITO”) or 20 μL of respiration buffer only (“− MITO”) and equilibrated in a non-CO₂ incubator for 10 minutes. A “Mitochondrial Stress Test” was then performed using a Seahorse instrument with 10 uM oligomycin, 20 uM FCCP, and 5 uM rotenone/antimycin A (Rot/AA). As shown in FIG. 1, porcine mitochondria treatment increased OCR at baseline (43.6% increase), oligomycin-treated HPAEC (204.9% increase), FCCP-treated HPAEC (8.4% increase), and Rot/AA-treated HPAEC (34.1% increase) in comparison to the corresponding baseline, oligomycin-treated, FCCP-treated, or Rot/AA-treated “− MITO” HPAEC control.

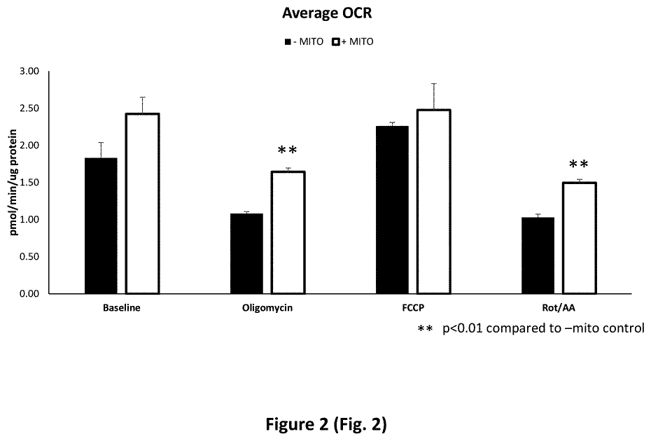

The effect of porcine mitochondria treatment of HPAEC on OCR after chronic cold exposure was also examined. HPAEC were placed in 4° C. for 12 hours. HPAEC recovered in normoxia for 1 hour at 37° C. in the presence of either 20 uL of mitochondria suspension (respiration buffer containing 172 particles per cell; “+ MITO”) or 20 μL of respiration buffer only (“− MITO”) and equilibrated in a non-CO₂ incubator for 50 minutes. HPAEC were rested in the Seahorse instrument at 37° C. under non-CO₂ conditions. A “Mitochondrial Stress Test” was then performed with the Seahorse instrument with 10 uM oligomycin, 20 uM FCCP, and 5 uM rotenone/antimycin A (Rot/AA). As shown in FIG. 2, porcine mitochondria treatment increased OCR at baseline (32.4% increase), oligomycin-treated HPAEC (51.9% increase), FCCP-treated HPAEC (9.5% increase), and Rot/AA-treated HPAEC (45.2% increase) in comparison to the corresponding baseline, oligomycin-treated, FCCP-treated, or Rot/AA-treated “− MITO” HPAEC control.

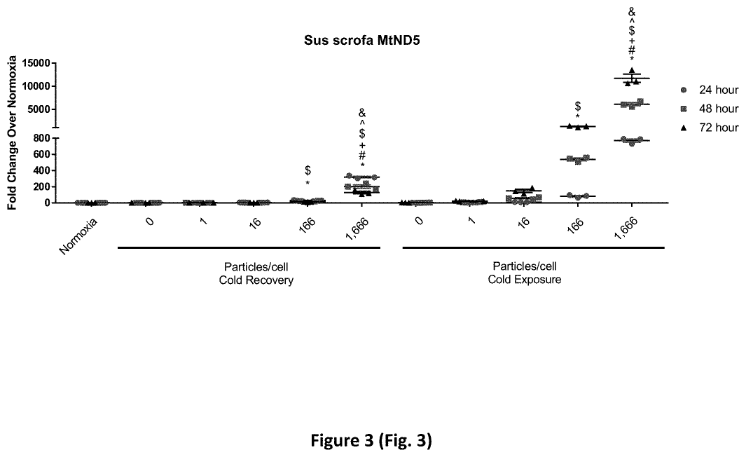

The uptake of porcine mitochondria by HPAEC exposed to cold stress was evaluated using a probe specific for porcine mitochondria (Sus scrofa) ND5 (MtND5). In particular, the effects of porcine mitochondria treatment during cold stress and during cold recovery were evaluated. Porcine mitochondria were administered to HPAEC undergoing cold stress. For the cold recovery group, HPAEC were cultured for 24 hours in normothermia and then for 24 hours at 4° C. prior to porcine mitochondria treatment. After porcine mitochondria treatment, the cold recovery HPAEC were incubated under recovery conditions (normothermia at 37° C.) for 24 hours, 48 hours, or 72 hours prior to harvest. For the cold exposure group, cells were cultured in normothermia for 48 hours, treated with porcine mitochondria, and immediately placed in 4° C. The cold exposure HPAEC were harvested after 24, 48, or 72 hours of cold exposure. cDNA was generated for each sample, and a primer/probe mixture was used to interrogate the expression levels of porcine MtNND5 (forward primer sequence CAGCACTATGTGCAATCACACAAAA; reverse primer sequence TGGTTGATGCCGATTGTCACTATT; reporter sequence TCGTAGCCTTCTCAACTTC; context sequence CAGCACTATGTGCAATCACACAAAA) as compared to the reference gene PPIA. As shown in FIG. 3, HPAEC under cold stress took up the porcine mitochondria in a dose-dependent manner, and maximal expression of porcine MtND5 was achieved at 1,666 particles per cell. In the cold recovery condition, maximal expression of porcine MtND5 was achieved at 24 hours, where a 26,201% increase in porcine MtND5 was observed compared to the untreated cold-recovery control. In the cold exposure condition, maximal expression of porcine MtND5 was achieved at 72 hours where a 301,932% increase in MtND5 was observed compared to the untreated cold-exposure control.

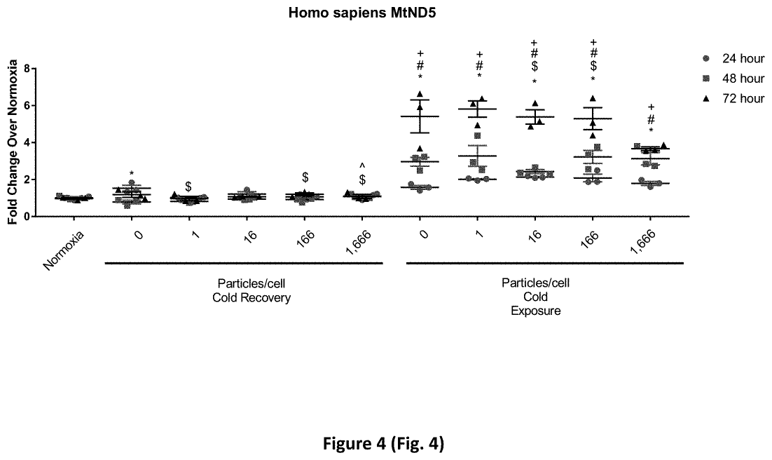

As shown in FIG. 4, transcription of human mitochondrial DNA in HPAEC exposed to cold stress was largely unaffected by porcine mitochondria treatment. HPAEC were treated, cultured under cold recovery or cold exposure conditions, and harvested at 24-hour, 48-hour, or 72-hour time points as described above. As determined using a probe specific for human MtND5, untreated control HPAEC under cold recovery conditions demonstrated a 55% increase in human MtND5 expression compared to normothermia controls. This increase was moderated by porcine mitochondria treatment, where 1 particle/cell demonstrated a 3.8% reduction in expression compared to untreated normothermia HPAEC and a 33% reduction in expression compared to the untreated cold-recovery control. In the cold exposure group, maximal expression of human MtND5 was achieved at 72 hours, but this increase was not significantly impacted by porcine mitochondria treatment.

Altogether, these findings show that human endothelial cells exposed to cold stress take up porcine mitochondria, which increases the rate of cellular oxygen consumption after cold injury and does not affect transcription of human mitochondrial RNA. In comparison to oligomycin-treated “− MITO” HPAEC controls, porcine mitochondria treatment increased OCR in oligomycin-treated HPAEC after acute and chronic cold exposure (FIGS. 1 and 2). These data indicate that mitochondria treatment increases proton leak respiration (i.e., the process wherein protons migrate into the matrix without producing ATP). Studies suggest that proton leak respiration decreases mitochondrial reactive oxygen species (ROS) production and that this leak or uncoupling protects against ROS in various diseases. See, e.g., Ganote, C. E. and S. C. Armstrong, J Mol Cell Cardiol. 2003, 35(7):749-59; Speakman, J. R., et al., Aging Cell. 2004, 3(3):87-95; and Green, K., et al., Diabetes. 2004, 53(Suppl. 1):S110-8. Therefore, porcine mitochondria treatment could protect against ROS in various diseases, such as diabetes and cardiovascular disease.

### Example 2

### Treatment of Cells with Porcine Mitochondria During Cold Recovery and Cold Exposure Alters the Expression of Genes Associated with Inflammation, the Innate Immune Response, and Cell Stress

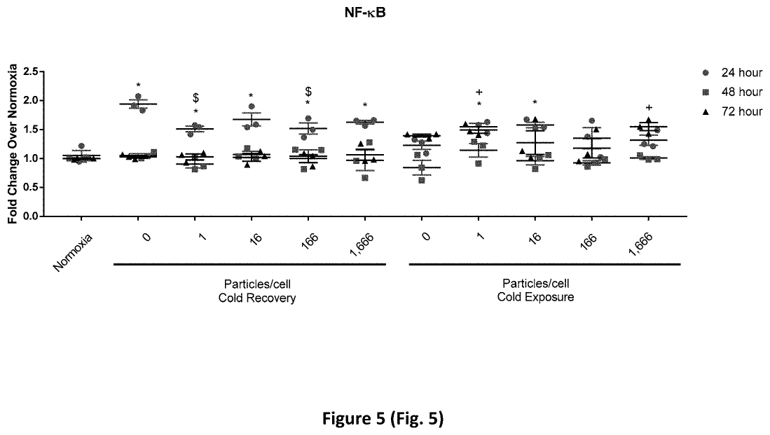

NF-κB is a transcription factor known to upregulate pro-inflammatory gene expression. The effects of porcine mitochondria treatment on NF-κB gene expression in HPAEC under cold exposure and cold recovery conditions was evaluated by qRT-PCR. As shown in FIG. 5, porcine mitochondria treatment of HPAEC reduces NF-κB expression in cold recovery at 24 hours. HPAEC were treated, cultured under cold recovery or cold exposure conditions, and harvested at 24-hour, 48-hour, or 72-hour time points as described above for FIG. 3. In the cold recovery condition, untreated control HPAEC demonstrated an 83% increase in NF-κB expression at 24 hours compared to normothermia controls. Porcine mitochondria treatment trended to decrease the NF-κB expression compared to untreated cold-recovery control HPAEC, with 1 particle/cell demonstrating a 22% decrease compared to untreated cold-recovery control HPAEC. In the cold exposure condition, a slight increase in NF-κB expression occurred at 24 hours in HPAEC treated with porcine mitochondria, but this increase is not statistically significant. These data suggest that porcine mitochondria treatment of human endothelial cells reduces a pro-inflammatory response associated with recovery from cold exposure.

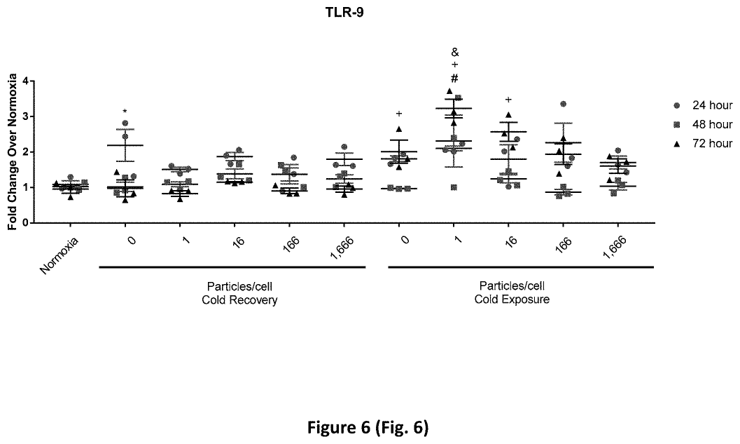

Toll-like receptor-9 (TLR-9) activates the innate immune response upon recognizing cytosolic mitochondrial DNA (mtDNA), which is a sign of cell damage. As shown in FIG. 6, porcine mitochondria treatment of HPAEC decreased TLR-9 expression in cold recovery after 24 hours. HPAEC were treated, cultured under cold recovery or cold exposure conditions, and harvested at 24-hour, 48-hour, or 72-hour time points as described above for FIG. 3. In the cold recovery condition, untreated control HPAEC demonstrated a 101% increase in TLR-9 expression at 24 hours compared to normothermia controls. Porcine mitochondria treatment trended to decrease the TLR-9 expression compared to untreated cold-recovery control HPAEC, with 166 particles/cell demonstrating a 37% decrease compared to untreated cold-recovery control HPAEC. In cold exposure conditions, maximal expression of TLR-9 occurs in HPAEC treated with 1 particle/cell, where a 60% increase in TLR-9 expression was observed compared to the untreated cold-exposure control HPAEC. These data suggest that, during cold recovery, porcine mitochondria treatment of human endothelial cells reduces the innate immune response associated with cell damage from cold exposure.

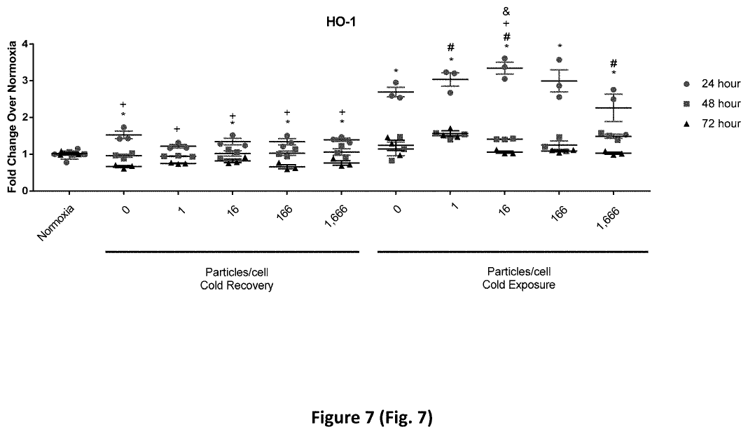

Upregulation of heme oxygenase-1 (HO-1) reduces inflammation and tissue damage and is cryoprotective during cellular stress. As shown in FIG. 7, porcine mitochondria treatment of HPAEC impacts the expression of HO-1. HPAEC were treated, cultured under cold recovery or cold exposure conditions, and harvested at 24-hour, 48-hour, or 72-hour time points as described above for FIG. 3. Porcine mitochondria treatment increased HO-1 expression in the cold exposure condition. Porcine mitochondria treatment was maximally effective at 16 particles/cell, where a 24% increase in HO-1 expression was seen compared to untreated cold-exposure control HPAEC (242% increase compared to untreated normothermia control HPAEC). These data suggest that porcine mitochondria treatment of human endothelial cells reduces inflammation and cell damage during cold exposure by increasing HO-1 expression.

### Example 3

### Treatment of Cells with Porcine Mitochondria Under Hypoxic Conditions Decreases Secretion of Pro-Inflammatory Gene Products

To evaluate the effects of porcine mitochondria treatment on human endothelial cells under hypoxic conditions, HPAEC were cultured at normoxia or hypoxia (1% O₂) for 24 hours prior to porcine mitochondria treatment. After porcine mitochondria treatment, HPAEC were placed back in their respective conditions, normoxia or hypoxia. 300 μL cell culture media was then collected at 24 hours, 48 hours, or 72 hours and placed in a sterile 1.5 mL Eppendorf tube at the appropriate time point (24 hours, 48 hours, or 72 hours). The tubes were spun down for 10 minutes at 4° C. at 2,000 rpm. The supernatant (270 μL) was collected and placed in a fresh, sterile 1.5 mL Eppendorf tube. These samples were immediately stored at −80° C. until analysis by inflammatory cytokine array. Secreted pro-inflammatory gene products were measured in the cell culture media using an inflammatory cytokine array (RayBiotech; Norcross, Ga.). A media-only control was utilized for background correction.

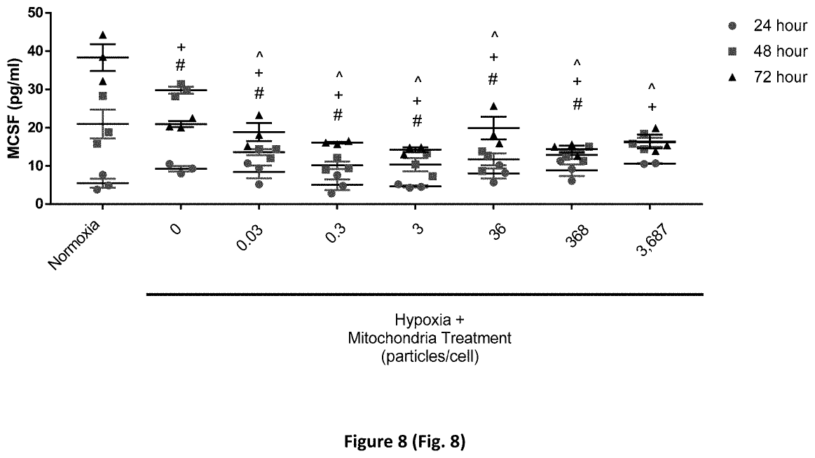

Macrophage-colony stimulating factor (M-CSF), also known as colony stimulating factor-1 (CSF-1), promotes healing but also promotes macrophages with an M1 phenotype. In ischemia/transplantation models, M-CSF serum levels spike during acute rejection of a transplanted organ. As shown in FIG. 8, assay by pro-inflammatory cytokine array showed that porcine mitochondria treatment of HPAEC decreased M-CSF secretion under hypoxic conditions. Porcine mitochondria treatment was maximally effective at 3 particles/cell, where M-CSF secretion was reduced by 65% compared to untreated hypoxia control HPAEC at 48 hours.

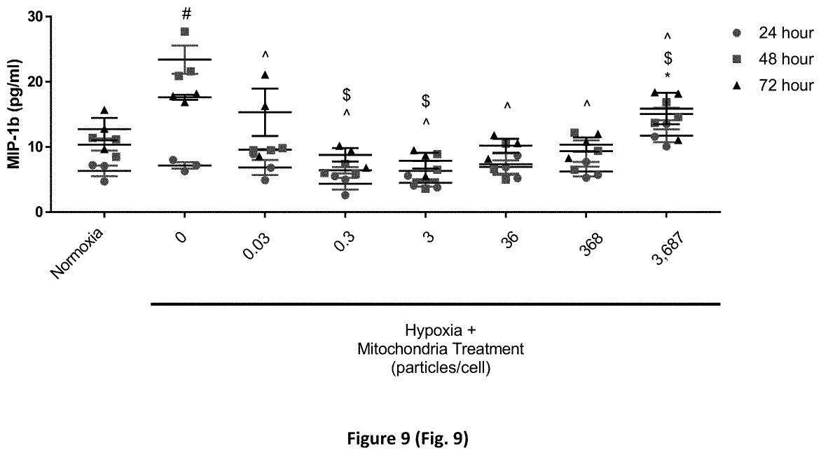

Macrophage inflammatory protein-1β (MIP-1β), also known as chemokine (C-C motif) ligand 4 (CCL4), is crucial for the immune response to infection and inflammation. MIP-1β activates immune cells, leading to acute inflammation, and can induce the synthesis and release of pro-inflammatory cytokines, such as IL-1β, IL-6, and TNF-α. As shown in FIG. 9, assay by pro-inflammatory cytokine array showed that porcine mitochondria treatment of HPAEC decreased MIP-1β secretion under hypoxic conditions. Porcine mitochondria treatment was maximally effective in reducing MIP-1β secretion at 3 particles/cell, where MIP-1β secretion was reduced by 73% compared to untreated hypoxia control HPAEC at 48 hours. A decreased in potency was seen at 3,687 particles/cell.

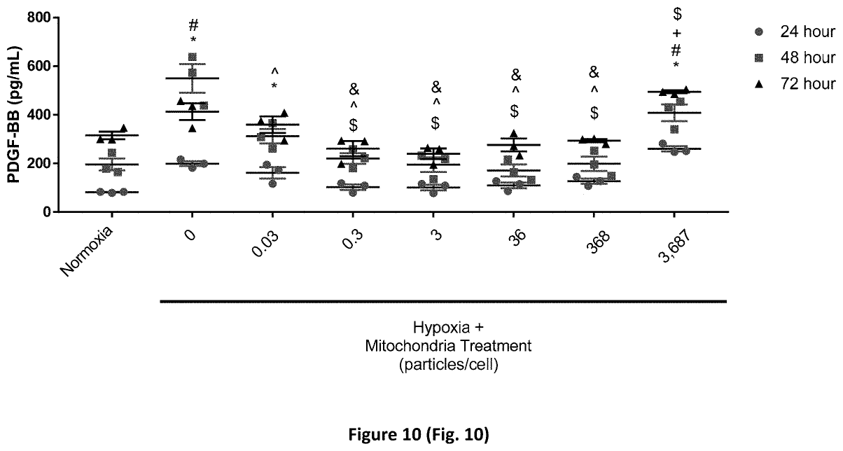

Platelet-derived growth factor-BB (PDGF-BB) is a potent inducer of pro-inflammatory cytokine production and stimulates the proliferation of cells. As shown in FIG. 10, assay by pro-inflammatory cytokine array showed that porcine mitochondria treatment of HPAEC decreased PDGF-BB secretion under hypoxic conditions. Porcine mitochondria treatment was maximally effective in reducing PDGF-BB secretion at 36 particles/cell, where PDGF-BB secretion was reduced by 69% compared to untreated hypoxia control HPAEC at 48 hours. A decrease in potency was seen at 3,687 particles/cell.

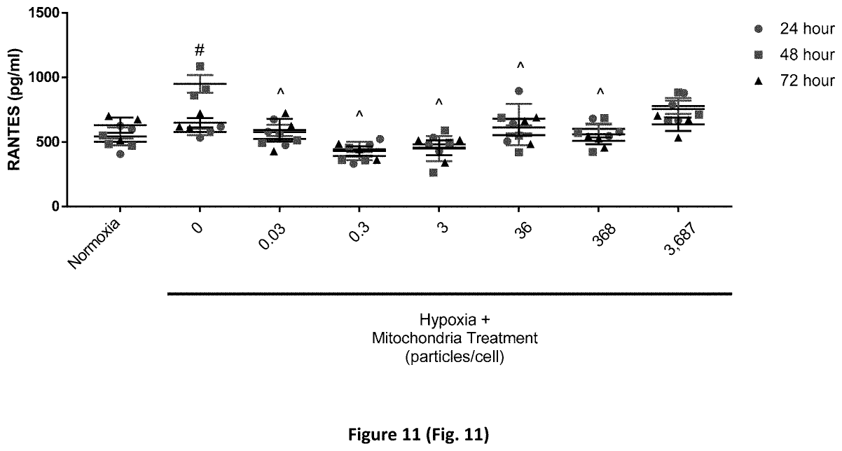

RANTES, also known as chemokine (C-C motif) ligand 5 (CCL5), is a pro-inflammatory chemokine that is upregulated by the NF-κB pathway. RANTES plays an active role in recruiting leukocytes to sites of inflammation. As shown in FIG. 11, assay by pro-inflammatory cytokine array showed that porcine mitochondria treatment of HPAEC decreased RANTES secretion under hypoxic conditions. Porcine mitochondria treatment was maximally effective in reducing RANTES secretion at 0.3 particles/cell, where RANTES secretion was reduced by 59% compared to untreated hypoxia control HPAEC at 48 hours. A decrease in potency was seen at 3,687 particles/cell.

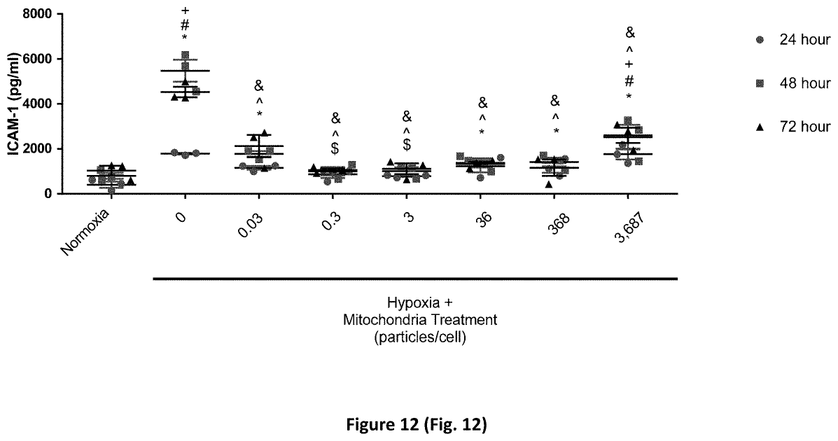

In inflammatory states, intracellular adhesion molecule-1 (ICAM-1) is upregulated to allow for passage of immune cells to the site of injury. ICAM-1 expression maintains a pro-inflammatory environment to allow for transmigration of immune cells. As shown in FIG. 12, assay by pro-inflammatory cytokine array showed that porcine mitochondria treatment of HPAEC decreased ICAM-1 secretion under hypoxic conditions. Porcine mitochondria treatment was maximally effective in reducing ICAM-1 secretion at 0.3 particles/cell, where ICAM-1 secretion was reduced by 82% compared to untreated hypoxia control HPAEC at 48 hours. A decrease in potency was seen at 3,687 particles/cell.

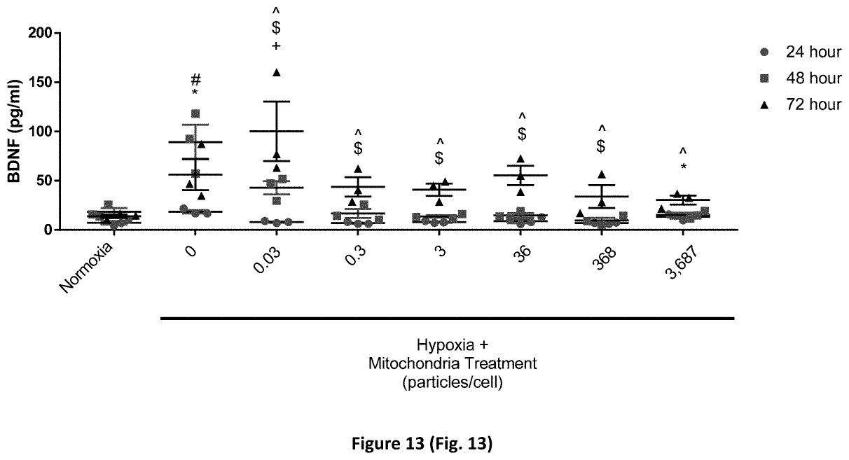

Brain-derived neurotrophic factor (BDNF) is known to be secreted by HPAEC after hypoxia exposure and may play a role in PAH pathogenesis. As shown in FIG. 13, assay by pro-inflammatory cytokine array showed that porcine mitochondria treatment of HPAEC decreased BDNF secretion under hypoxic conditions. Porcine mitochondria treatment was maximally effective in reducing BDNF secretion at 3 particles/cell, where BDNF secretion was reduced by 85% compared to untreated hypoxia control HPAEC at 48 hours.

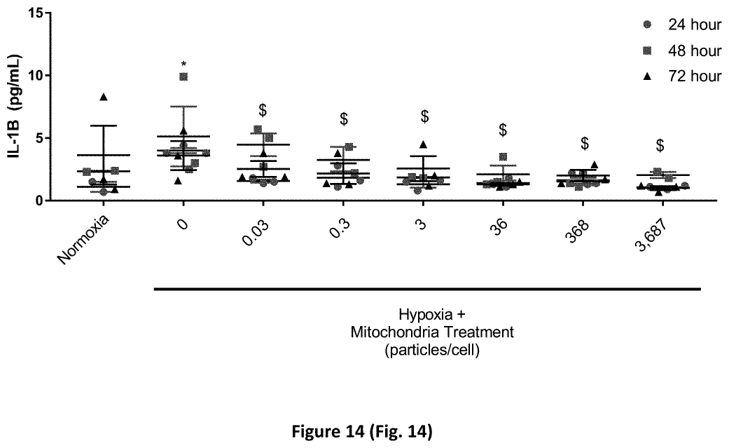

Interleukin-1β (IL-1β) is a pro-inflammatory cytokine implicated in inflammation. Expression of IL-1β is regulated by the inflammasome. As shown in FIG. 14, assay by pro-inflammatory cytokine array showed that porcine mitochondria treatment of HPAEC decreased IL-1β secretion under hypoxic conditions. Porcine mitochondria treatment was maximally effective in reducing IL-1β secretion at 368 particles/cell, where IL-1β secretion was reduced by 70% compared to untreated hypoxia control HPAEC at 48 hours.

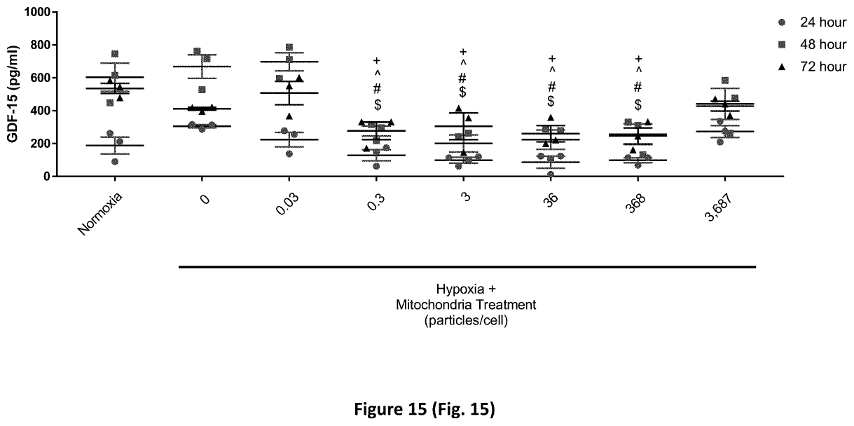

Growth/differentiation factor 15 (GDF15) is a member of the TGF-β superfamily. In the lung, overexpression of GDF15 leads to an exaggerated immune response, while suppression of GDF15 expression attenuates the inflammatory response. As shown in FIG. 15, assay by pro-inflammatory cytokine array showed that porcine mitochondria treatment of HPAEC decreases GDF15 secretion under hypoxic conditions. Porcine mitochondria treatment was maximally effective in reducing GDF15 secretion at 3 particles/cell, where GDF15 secretion was reduced by 70% compared to untreated hypoxia control HPAEC at 48 hours.

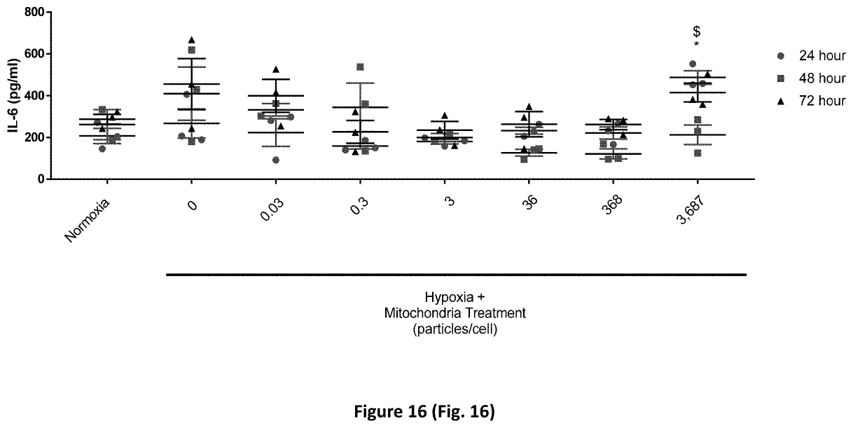

Interleukin-6 (IL-6) is a pleiotropic cytokine produced in response to tissue damage. IL-6 plays a role in inflammation and immune cell activation. As shown in FIG. 16, assay by pro-inflammatory cytokine array showed that porcine mitochondria treatment of HPAEC decreased IL-6 secretion under hypoxic conditions. Porcine mitochondria treatment was maximally effective in reducing IL-6 secretion at 368 particles/cell, where IL-6 secretion was reduced by 70% compared to untreated hypoxia control HPAEC at 48 hours.

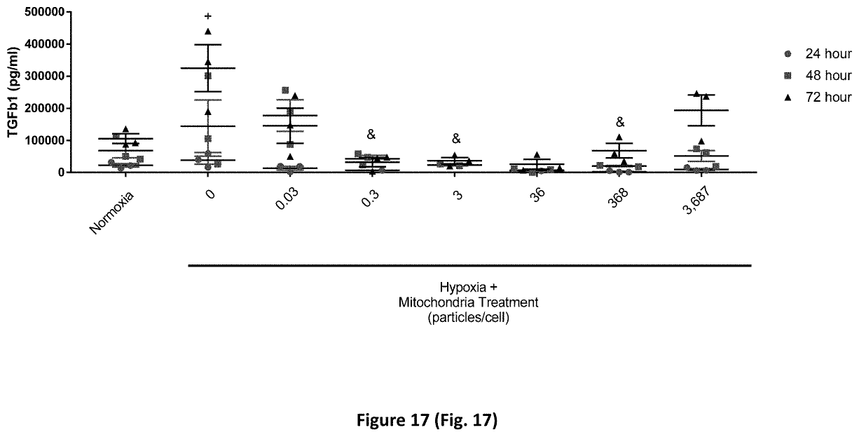

Transforming growth factor-β1 (TGF-β1) is a pleiotropic cytokine with potent regulatory and inflammatory activity. In the presence of IL-6, TGF-β1 is known to drive the differentiation of T-helper 17 (Th17) cells, which promote an inflammatory environment. As shown in FIG. 17, assay by pro-inflammatory cytokine array showed that porcine mitochondria treatment of HPAEC decreased transforming growth factor-01 (TGF-β1) secretion under hypoxic conditions. Porcine mitochondria treatment was maximally effective in reducing TGF-β1 secretion at 36 particles/cell, where TGF-β1 secretion was reduced by 95% compared to untreated hypoxia control HPAEC at 48 hours.

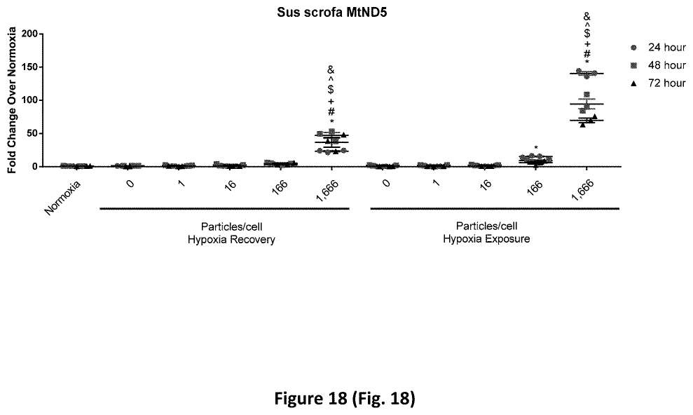

The uptake of porcine mitochondria by HPAEC exposed to hypoxic stress was evaluated using the probe specific for porcine MtND5. In particular, the effects of porcine mitochondria treatment during hypoxia exposure and during hypoxia recovery were evaluated. Porcine mitochondria were administered to HPAEC undergoing hypoxic stress. For the hypoxia recovery group, HPAEC were cultured for 24 hours in normoxia and then for 24 hours in hypoxia (1% O₂) prior to porcine mitochondria treatment. After porcine mitochondria treatment, the hypoxia recovery HPAEC were placed back in normoxia for 24 hours, 48 hours, or 72 hours prior to harvest. For the hypoxia exposure group, HPAEC were cultured in normoxia for 48 hours, treated with porcine mitochondria, and immediately placed in hypoxia (1% O₂). The hypoxia exposure HPAEC were harvested after 24, 28, or 72 hours of hypoxia exposure. As determined using the probe specific for porcine MtND5 and shown in FIG. 18, HPAEC under hypoxic stress took up the porcine mitochondria in a dose-dependent manner, and maximal expression of porcine MtND5 was achieved at 1,666 particles per cell. In the hypoxia recovery condition, maximal expression of porcine MtND5 was achieved at 48 hours, where a 4,655% increase in porcine mtND5 was observed compared to the untreated hypoxia-recovery control. In the hypoxia exposure condition, maximal expression was achieved at 24 hours, where a 26,680% increase in porcine mtND5 was observed compared to the untreated hypoxia-exposure control.

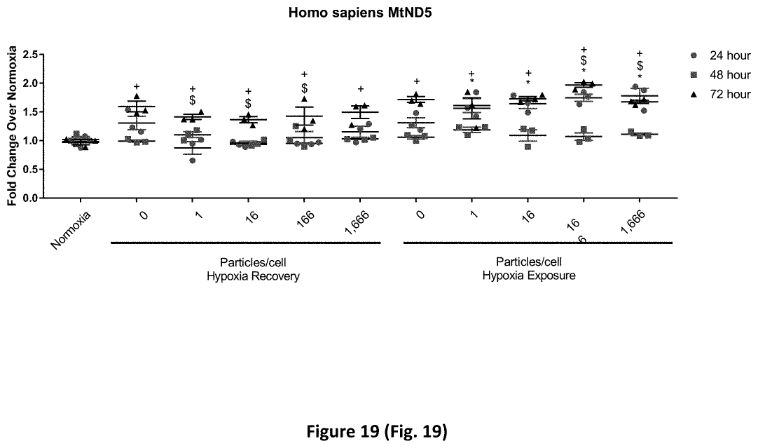

As shown in FIG. 19, transcription of human mitochondrial DNA in HPAEC exposed to hypoxic stress was largely unaffected by porcine mitochondria treatment. HPAEC were treated, cultured under hypoxia recovery or hypoxia exposure conditions, and harvested at 34-hour, 48-hour, or 72-hour time points as described above for FIG. 18. As determined using the probe specific for human MtND5, maximal expression of human MtND5 for both the hypoxia recovery group and the hypoxia exposure group occurred at 72 hours. The time point that appeared impacted by porcine mitochondria treatment occurred at 24 hours. In the hypoxia recovery group, there was a trend for decreased human MtND5 expression in HPAEC treated with porcine mitochondria, with 1 particle/cell demonstrating a 33% reduced expression compared to untreated hypoxic controls at 24 hours. In the hypoxia exposure group, there was a trend for increased human MtND5 expression in HPAEC treated with porcine mitochondria, with 1,666 particles/cell resulting in a 36% increase compared to untreated hypoxia-exposure cells at 24 hours.

Altogether, these results show that treatment of human endothelial cells exposed to hypoxic conditions take up porcine mitochondria, which decreases secretion of a wide array of pro-inflammatory gene products and does not affect transcription of human mitochondrial RNA. These results suggest that porcine mitochondria treatment is an effective means to reduce the inflammation and cell damage associated with hypoxia during organ or tissue transplantation, as well as during cold storage or transport of cells, tissues, and organs.

### Example 4

### Treatment of Cells with Porcine Mitochondria Alters Gene Expression Under Hypoxic Conditions

To evaluate the effects of porcine treatment on gene expression under hypoxic conditions, porcine mitochondria were administered to HPAEC undergoing hypoxic stress. For the hypoxia recovery group, HPAEC were cultured for 24 hours in normoxia and then for 24 hours in hypoxia (1% O₂) prior to porcine mitochondria treatment. After porcine mitochondria treatment, the hypoxia recovery HPAEC were placed back in normoxia for 24 hours, 48 hours, or 72 hours prior to harvest. For the hypoxia exposure group, HPAEC were cultured in normoxia for 48 hours, treated with porcine mitochondria, and immediately placed in hypoxia (1% O₂). The hypoxia exposure HPAEC were harvested after 24, 28, or 72 hours of hypoxia exposure. Gene expression was evaluated by qRT-PCR.

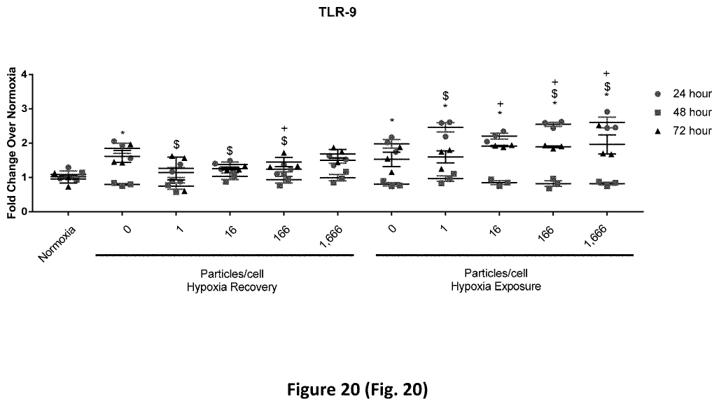

As discussed above, toll-like receptor-9 (TLR-9) activates the innate immune response upon recognizing cytosolic mtDNA, which is a sign of cell damage. Porcine mitochondria treatment of HPAEC reduced TLR-9 expression in hypoxia recovery but increased TLR-9 expression in hypoxia exposure at 24 hours, as shown in FIG. 20. In the hypoxia recovery group, there was a trend for decreased TLR-9 expression in HPAEC treated with porcine mitochondria, with 1 particle/cell demonstrating a 38% reduced expression compared to untreated hypoxic controls at 24 hours post-treatment. In the hypoxia exposure group, there was a trend for increased TLR9 expression in HPAEC treated with porcine mitochondria, with 1,666 particles/cell resulting in a 32% increase compared to untreated hypoxia-exposure cells at 24 hours post-treatment. These data suggest that porcine mitochondria treatment of human endothelial cells reduces the innate immune response associated with cell damage during hypoxia recovery.

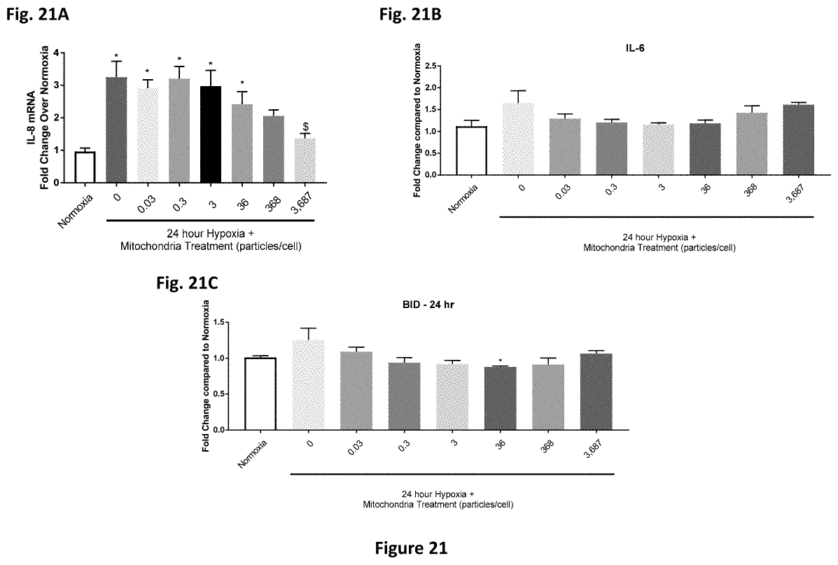

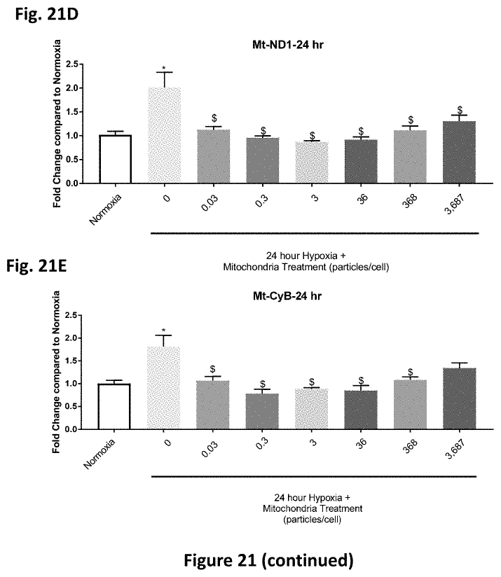

Interleukin-8 (IL-8; CXCL8) attracts and activates neutrophils in inflammatory regions. Elevation of IL-8 is an indicator for graft failure and other pathological outcomes. As discussed above, IL-6 is a pleiotropic cytokine that is produced in response to tissue damage and that plays a role in inflammation and immune cell activation. As shown in FIG. 21, porcine mitochondria treatment of HPAEC undergoing hypoxic stress reduced mRNA expression of IL-8 and IL-6. Porcine mitochondria treatment of hypoxic HPAEC was maximally effective for reducing IL-8 expression at 3,687 particles/cell, where a 58% decrease in IL-8 expression was seen compared to untreated hypoxic controls (FIG. 21A). Porcine mitochondria treatment of hypoxic HPAEC is maximally effective for reducing IL-6 expression at 3 particles/cell, where a 30% decrease in IL-6 expression was seen compared to untreated hypoxic controls (FIG. 21B). These data suggest that porcine mitochondria treatment of human endothelial cells reduces an inflammatory cytokine response associated with cells undergoing hypoxic stress and tissue damage.

BH3 interacting-domain death agonist (BID) is a pro-apoptotic protein that plays a role in disrupting the outer mitochondrial membrane in response to apoptosis signaling. As shown in FIG. 21, porcine mitochondria treatment of HPAEC undergoing hypoxic stress reduced mRNA expression of BID. Porcine mitochondria treatment of hypoxic HPAEC is maximally effective for reducing BID expression at 36 particles/cell, where a 30% decrease in BID expression was seen compared to untreated hypoxic controls (FIG. 21C). These data suggest that porcine mitochondria treatment of human endothelial cells reduces mitochondrial membrane disruption associated with apoptosis induced by hypoxic stress.

Mitochondrial ND1 (MtND1) is a gene involved in the respiratory complex 1, which is the first step in the electron transport chain of mitochondrial oxidative phosphorylation. Mitochondrial cytochrome B (MtCyB) is the only mitochondrial encoded subunit of respiratory complex III. As shown in FIG. 21, porcine mitochondria treatment of HPAEC undergoing hypoxic stress reduced mRNA expression of MtND1 and MtCyB. Porcine mitochondria treatment of hypoxic HPAEC is maximally effective for reducing human MtND1 expression at 3 particles/cell, where a 57% decrease in MtND1 expression was seen compared to untreated hypoxic controls (FIG. 21D). Porcine mitochondria treatment of hypoxic HPAEC is maximally effective for reducing human MtCyB expression at 0.3 particles/cell, where a 57% decrease in MtCyB expression was seen compared to untreated hypoxic controls (FIG. 21E).

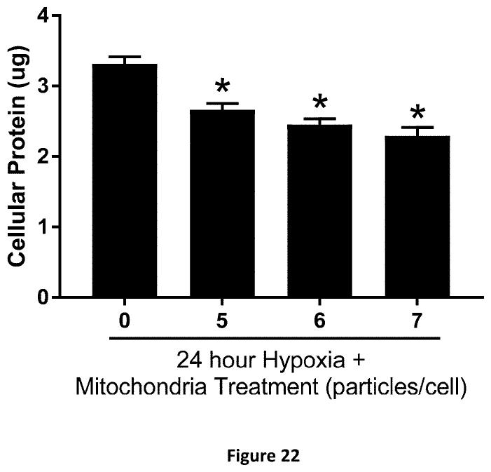

A 24-hour hypoxia exposure reduces host cell mitochondrial activity and thus produces a compensatory stress response that increases mitochondrial gene expression (e.g., increased ND1 and CyB expression). Porcine mitochondrial transplant protects the host cell from hypoxia stress, eliminating the need for genetic compensation. It was also found that treatment of human endothelial cells with porcine mitochondria decreased hypoxia-induced cell proliferation as indicated by a decrease in total cellular protein content of mitochondria treated HPAEC (FIG. 22). Aberrant endothelial cell proliferation is implicated in the pathogenesis of pulmonary hypertensive disease, including pulmonary arterial hypertension. See, e.g., Sakao, S. et al., Respir Res. 2009; 10(1):95. Therefore, these data suggest that in vivo or ex vivo treatment of a patient's cells with porcine mitochondria could treat lung disease or alleviate symptoms associated with lung disease.

### Example 5

### Treatment of Epithelial Cells with Porcine Mitochondria Improves Nucleic Acid Content

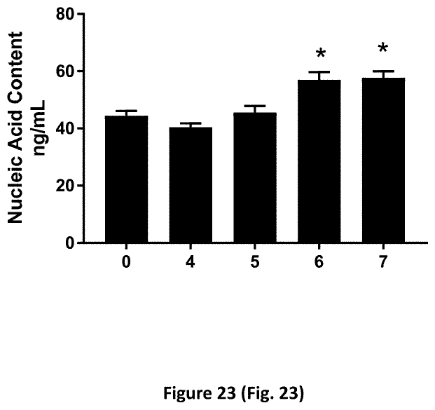

Human alveolar epithelial type II (AT2) cells have low viability coming out of cryo-storage and do not adhere well to cell culture plates. To determine whether porcine mitochondria treatment improves cellular viability and adherence of human lung epithelial cells after cryopreservation, AT2 cells were seeded directly from cryo-storage with and without porcine mitochondria and incubated overnight in a standard incubator. Following overnight incubation, the nucleic acid content of AT2 cells treated with porcine mitochondria increased by 23% compared to the untreated AT2 cell control (FIG. 23). These data show that porcine mitochondria treatment improves cell viability, adhesion or growth of human lung epithelial cells after cryopreservation. Thus, porcine mitochondria treatment can be implemented as part of a cellular therapy to improve the storage, viability or functionality of lungs or lung cells.

### Example 6

### Stability and Functionality of Porcine Mitochondria are Retained Following Cold Storage

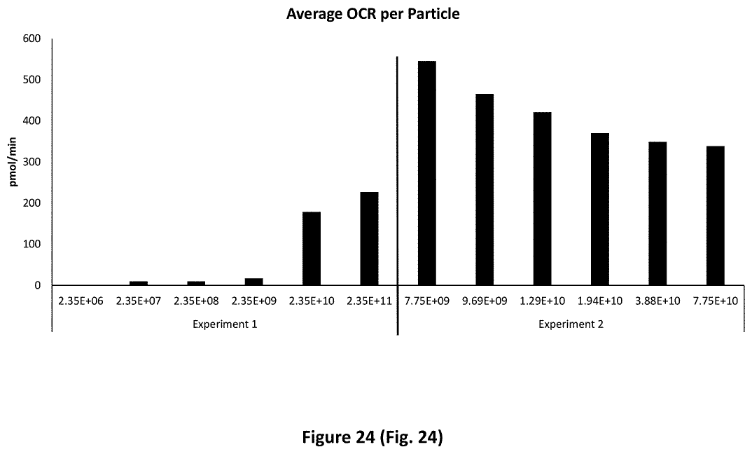

Two porcine mitochondrial isolations (“Experiment 1” and “Experiment 2”) were used to test mitochondrial activity in a Seahorse instrument. A series of mitochondrial dilutions were created in ADP-containing respiration buffer. 50 μL of each mitochondrial suspension was loaded into six wells of an 8-well Seahorse cell culture plate. The plate was centrifuged at 2000×g for 20 minutes at 4° C. After centrifugation, 200 μL of ADP-containing respiration buffer (RB) was added to each well, and the plate was equilibrated in the non-CO₂ incubator for 10 minutes. Baseline oxygen consumption was recorded with the Seahorse instrument. FIG. 24 shows the mitochondrial activity of isolated porcine mitochondria at various concentrations in respiration buffer containing adenosine diphosphate (ADP). A “maxing out” of OCR at ˜7e⁹ particles (particles were counted using the Zetaview) was observed.

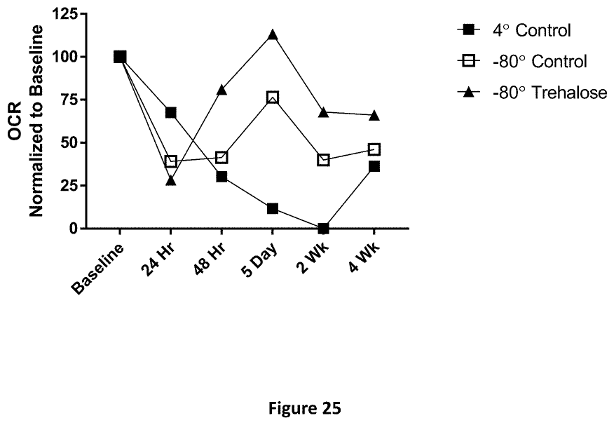

To determine whether porcine mitochondria retain mitochondrial activity after cold storage, three 200 μL aliquots of porcine mitochondria were centrifuged at 15,000×g for 10 minutes. The supernatant was removed, and two pellets were resuspended in 200 μL of ADP-containing respiration buffer (RB). One pellet was resuspended in 200 μL of trehalose (TH) storage buffer. One RB pellet was stored at 4° C. overnight, and the remaining RB and TH pellets were stored at −80° C. Approximately 22 hours after cold storage, the tubes were thawed on ice, and a 1:10 dilution was performed using respiration buffer. A 1:10 dilution was also used for the control group of freshly thawed mitochondria taken on the previous day (i.e., the porcine mitochondria of “Experiment 2” of FIG. 24). 50 μL of mitochondrial suspension was loaded into six wells of an 8-well Seahorse cell culture plate. The plate was centrifuged at 2000×g for 20 minutes at 4° C. After centrifugation, 200 μL of respiration buffer was added to each well, and the plate was equilibrated in the non-CO₂ incubator for 10 minutes. Baseline oxygen consumption was recorded with the Seahorse instrument. As shown in FIG. 25, porcine mitochondria retain mitochondrial activity after cold storage at −80° C. While mitochondria activity decreased at 4° C. over time, storage at −80° C. resulted in retention of approximately 40% OCR (mitochondrial activity). Storage in trehalose improved OCR, resulting in approximately 60% retention in original OCR rate.

### Example 7

### Porcine Mitochondria Treatment Improves Lung Function During EVLP

To prepare lungs for ex vivo lung perfusion (EVLP), porcine lungs were inflated, and the trachea clamped. Lungs were stored in ice cold saline for approximately 1 hour prior to initiation of procedure. The pulmonary artery (PA) and main bronchus (trachea) were cannulated at room temperature. The left atrium (LA) was kept open to allow for free efflux of perfusate from the lung. During the 4-hour EVLP, lungs were ventilated and perfused with 37° C. Steen solution, which was buffered with bicarbonate and deoxygenated with 5% CO₂, 95% N₂. Pressure controlled ventilation was used with airway pressure capped at 17 cmH₂O. PA perfusion was started at 100 ml/min and increased to 300 ml/min over approximately 30-45 minutes. Perfusion was held at 300 ml/min during the EVLP. Before mitochondria injection, gas exchange was assessed by measuring PA and LA P02 at an FiO₂ of 100%. Physiological parameters, such as dynamic compliance, were also recorded at this time. Mitochondria or respiration buffer (control) were then injected into the PA line and perfusion was stopped for 10 minutes to allow for mitochondrial uptake into the lung. Perfusion was resumed and EVLP assessments, including gas exchange, were made at 15 minutes, 1 hour, 2 hour and 4 hours post-injection.

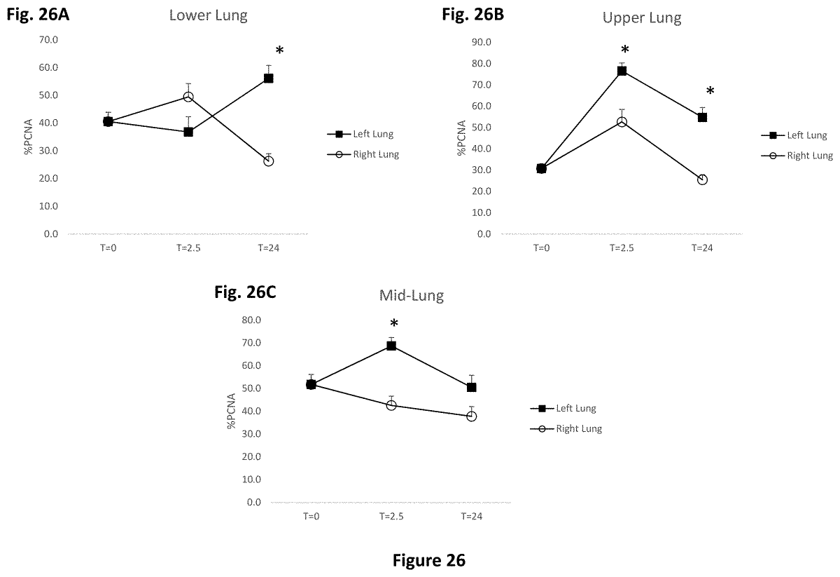

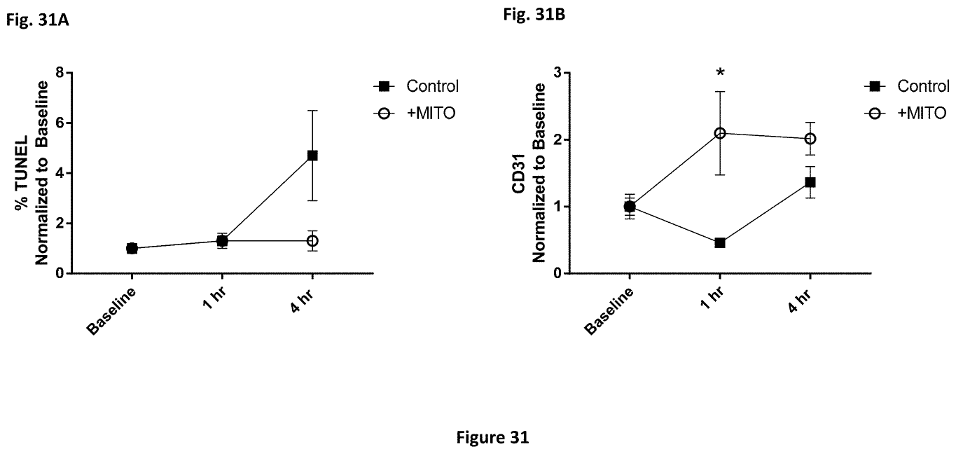

As shown in FIG. 26, porcine mitochondria treatment improved the function of an isolated porcine cadaveric lung while on EVLP. In comparison to the right lung control, isolated porcine mitochondria injected into the left lung increased proliferating cell nuclear antigen (PCNA) positive cells in the lower lung (FIG. 26A), upper lung (FIG. 26B), and mid-lung (FIG. 26C) as measured by histology (FIG. 26A). Porcine mitochondria treatment was maximally effective at 24 hours in the lower lung (FIG. 26A), where a 50% improvement was seen in porcine mitochondria-treated cells compared to control (arrow). As further shown in FIG. 31, injection of isolated porcine mitochondria into a porcine cadaveric lung on EVLP (“+ MITO”) decreases the percentage of apoptotic cells (% TUNEL; FIG. 31A) and increases expression of the cellular adhesion molecule CD31 (FIG. 31B) in comparison to a porcine cadaveric lung injected with respiration buffer (“Control”). The percentage of apoptotic cells was determined by TUNEL assay on tissue biopsies taken from the porcine cadaveric lungs during EVLP. CD31 expression was determined by immunofluorescence staining of tissue biopsies with an anti-CD31 antibody.

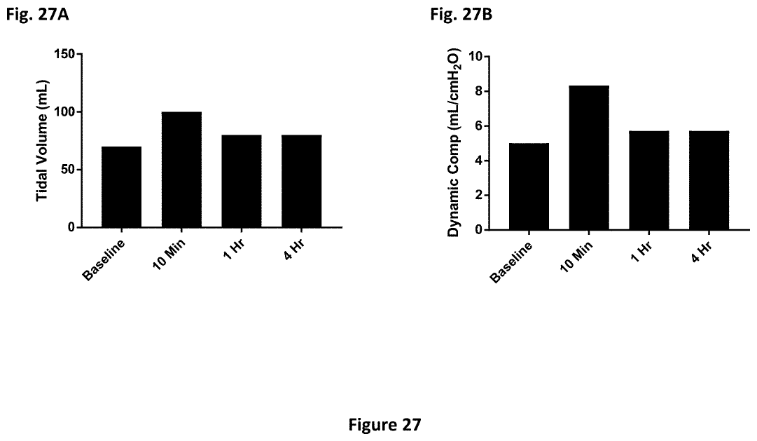

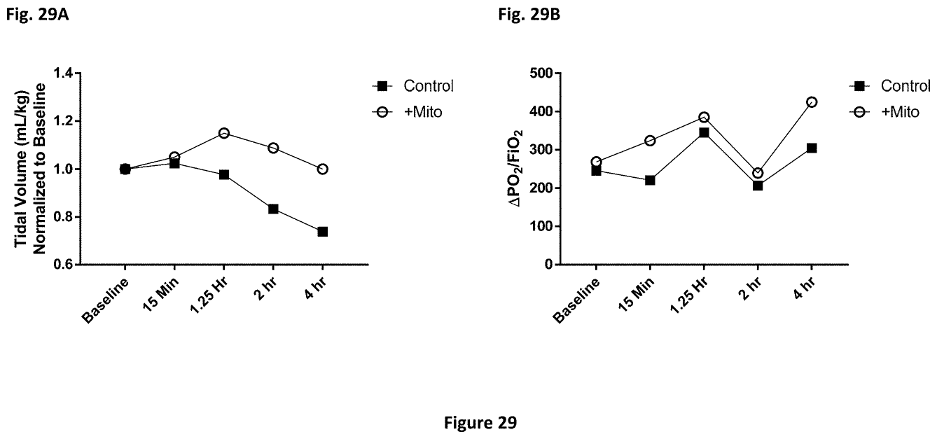

As shown in FIG. 27, porcine mitochondria treatment improved the parameters of tidal volume (FIG. 27A) and dynamic compression (FIG. 27B) of an isolated porcine cadaveric lung while on EVLP. Isolated porcine mitochondria were injected into an isolated porcine cadaveric lung on EVLP, and perfusion was turned off for 10 minutes while the lung continued inflation. Tidal volume (ml/kg) and dynamic compression (TV/(PIP-PEEP)) were determined at 10 minutes post-injection, 1 hour post-injection, and 4 hours post-injection (TV=tidal volume; PIP=peak inspiratory pressure; PEEP=positive end expiratory pressure). Baseline tidal volume and dynamic compression represent pre-injection tidal volume and dynamic compression, respectively. A 30% improvement in tidal volume and a 40% increase in dynamic compression are seen at 10 minutes post-injection in comparison to baseline. As further shown in FIG. 29, injection of isolated porcine mitochondria into a porcine cadaveric lung on EVLP increased tidal volume (mL/kg; FIG. 29A) and gas exchange (ΔPO₂/FiO₂; FIG. 29B) in comparison to a porcine cadaveric lung on EVLP injected with respiration buffer.

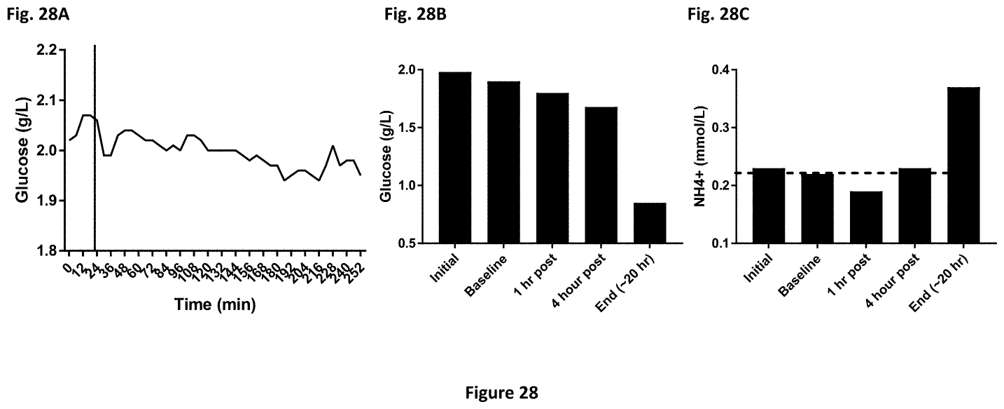

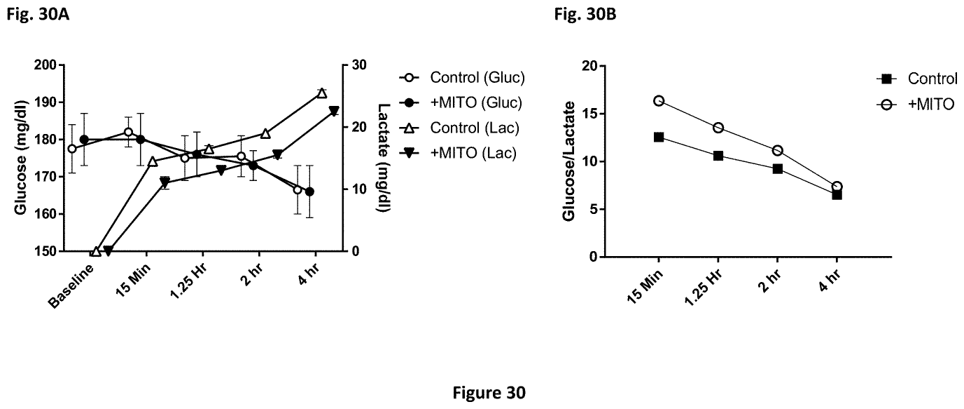

FIG. 28 shows that, following injection of isolated porcine mitochondria into an isolated porcine cadaveric lung on EVLP, there was an immediate and progressive drop in media glucose as well as a 17% decrease in circulating ammonium at one hour post-injection. An isolated porcine cadaveric lung on EVLP was injected with isolated porcine mitochondria 24 minutes after commencement of EVLP and maintained on EVLP for approximately 20 hours. Glucose (g/L) in the circulating media was quantitated using BioPat (Sartorius, Bohemia, N.Y.) (FIG. 28A) and Nova (Nova Biomedical, Waltham, Mass.) (FIG. 28B), and circulating ammonium (NH₄⁺; mmol/L) was quantitated using Nova (FIG. 28C). Initial Nova glucose and ammonium levels represent Nova glucose and ammonium levels at time 0 post-EVLP. Baseline Nova glucose and ammonium levels represent Nova glucose and ammonium levels immediately prior to injection of the porcine mitochondria. As further shown in FIG. 30, injection of isolated porcine mitochondria into a porcine cadaveric lung on EVLP (“+ MITO”) decreases the amount of circulating lactate (mg/ml; FIG. 30A), leading to an increased glucose/lactate ratio (FIG. 30B) in comparison to a porcine cadaveric lung on EVLP injected with respiration buffer.

Mitochondrial injection increased tidal volume and gas exchange during EVLP compared to respiration buffer control (FIG. 29). Mitochondrial injection decreased circulating lactate during EVLP, leading to an increase in the glucose/lactate ratio (FIG. 30). Lastly, tissue biopsies taken during EVLP revealed a decrease in TUNEL staining (apoptotic cells), and increase in CD31 (marker of cellular adhesion), during EVLP (FIG. 31).

Altogether, lungs treated with isolated porcine mitochondria during EVLP showed improved cellular function (e.g., increased cell viability, adhesion and growth), improved lung function (e.g., improved tidal volume, dynamic compression, and gas exchange), and improved metabolic activity (e.g., increased glucose/lactate ratio). Thus, porcine mitochondria treatment can be implemented during EVLP to improve lung viability and function.

### Example 8

### Rapid Assessment of the Health and Function of Isolated Porcine Mitochondria

Phenotypic characteristics of damaged mitochondria (e.g., swelling, dysregulated mPTP opening, reduced respiration, reduced membrane potential, and complete permeability) were used to rapidly assess the health of isolated porcine mitochondria. In particular, the phenotypic characteristics of isolations that yielded healthy mitochondria were compared to isolations that physically damaged the mitochondria (i.e., excess heat generation, extended exposure to enzyme) or mitochondria that were isolate from a fibrotic heart. In each case, the mitochondria were stored at −80° C. for 24 hours prior to analysis.

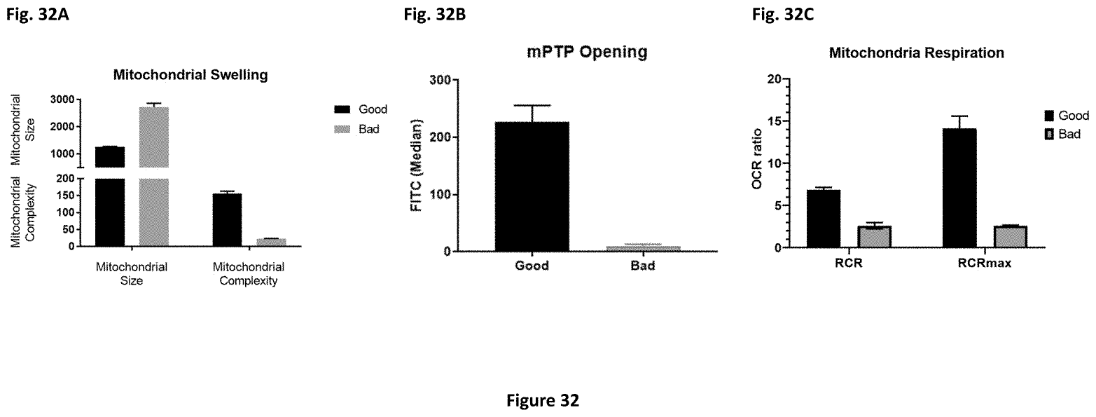

As shown in FIG. 32, the health and function of isolated mitochondria can be rapidly assessed by measuring mitochondrial swelling, mPTP opening, and/or mitochondria respiration. Mitochondria swelling was measured using flow cytometry. The mitochondria used in this study were stored at −80° C. for 24 hours prior to analysis. Unstained mitochondria are collected up to 30,000 events. Parameters assessed include forward side scatter area (FSC-A; size) and side scatter height (SSC-H; complexity). Mitochondria were determined to have a swelling phenotype if they had increased size and decreased complexity. Compared to healthy mitochondria, the damaged mitochondria were larger and less complex (FIG. 32A).

mPTP opening was measured using flow cytometry. Mitochondria were stained with 4 μM calcein-AM. Mitochondria were collected up to 30,000 events, excited with a 488 nm laser, and assessed on FITC emission. Mitochondria were determined to have a regulated mPTP if they were able to retain fluorescent calcein, resulting in FITC+ staining. Mitochondria were determined to have dysregulated, continuous mPTP opening if they were unable retain fluorescent calcein, resulting in reduced FITC staining. Compared to healthy mitochondria, the damaged mitochondria had drastically reduced FITC emission due to their inability to retain calcein AM (FIG. 32B).

To evaluate mitochondria respiration, respiratory control ratios (RCRs) were determined using the Seahorse instrument. RCRs were calculated from the oxygen consumption rate (OCR) during ADP-stimulated respiration (RCR) and uncoupled respiration (RCRmax). The OCR during each of these two states was divided by the basal OCR to obtain the OCR ratio. Maximal respiration was achieved by injecting the mitochondrial protonophore uncoupler BAM15. Compared to healthy mitochondria, the damaged mitochondria had dramatically reduced ADP-stimulated respiration rates and uncoupled respiration rates (FIG. 32C).

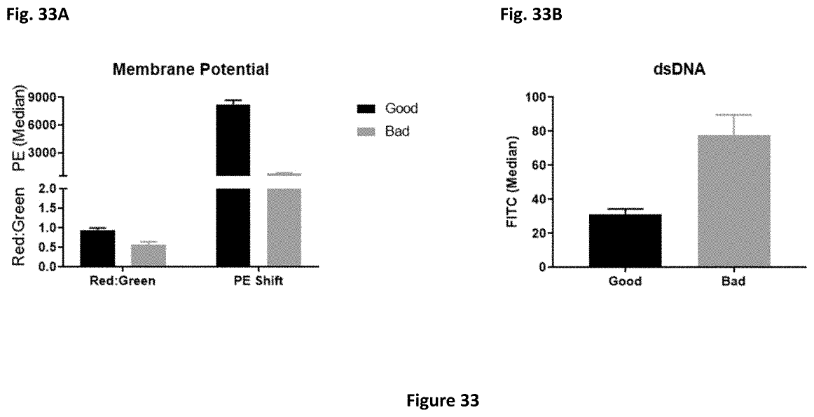

As shown in FIG. 33, the health and function of isolated mitochondria can also be rapidly assessed by measuring mitochondria membrane potential and/or mitochondria membrane permeability. Changes in mitochondria membrane potential were assessed by flow cytometry using a JC-1 assay. Mitochondria were stained with 2 μM JC-1, collected up to 30,000 events excited with a 488 nm laser, and assessed on FITC and PE emission. JC-1 dye exhibits potential-dependent accumulation in mitochondria, indicated by a fluorescent emission shift from green/FITC (˜529 nm) to red/PE (˜590 nm). The membrane potential-sensitive color shift is due to concentration-dependent formation of red fluorescent J-aggregates. Mitochondria depolarization is indicated by a decrease in the red:green fluorescence intensity ratio or by a decrease in the signal intensity in the PE (red) channel. Compared to healthy mitochondria, damaged mitochondria had a decreased red:green ratio and a drastically reduced PE emission (FIG. 33A).

Complete mitochondria permeability was measured by flow cytometry using a SYTOX green nucleic acid stain, which easily permeates mitochondria with comprised membranes. Mitochondria were stained with 1 μM SYTOX, excited with a 488 nm laser, collected up to 30,000 events, and assessed on FITC emission. Damaged mitochondria stained with SYTOX green will have higher FITC signal intensity than non-damaged mitochondria stained with SYTOX green. Compared to healthy mitochondria, the damaged mitochondria demonstrated increased FITC emission (FIG. 33B).

Altogether, these data show that the health and function of isolated porcine mitochondria can be rapidly assessed by measuring phenotypic characteristics of damaged mitochondria.

### Example 9

### Isolated Porcine Mitochondria Retain Mitochondrial Function after Cold Storage at −80° C.

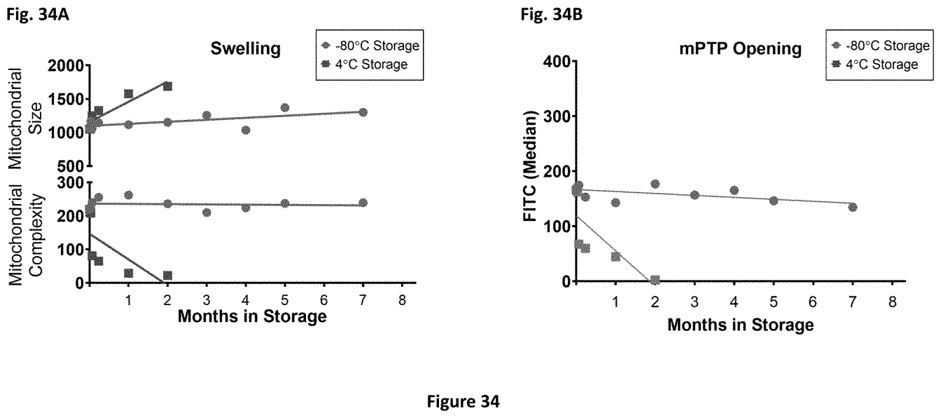

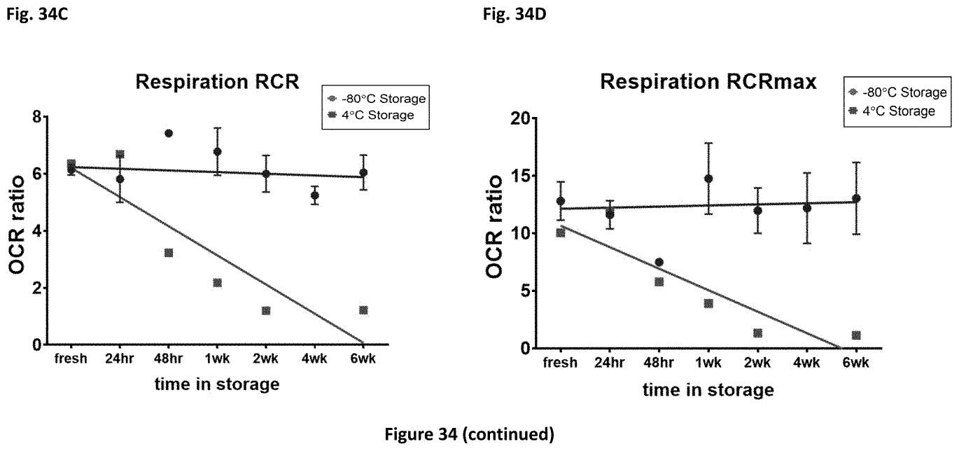

One central tenant reported in the literature pertaining to isolated mitochondria is the inability to store mitochondria in a way to preserve function. To test the ability to store mitochondria long term, characterization parameters (mitochondria swelling, mPTP opening, respiration, membrane potential, and complete permeability) were assessed in mitochondria that had been suspended in trehalose buffer (300 mM trehalose, 10 mM HEPES, 10 mM KCl, 1 mM EGTA, 0.1% fatty acid-free BSA, pH to 7.2) and stored at two conditions:
(1) mitochondria stored at 4° C., which was considered to be non-preserving to mitochondria function; and
(2) mitochondria stored at −80° C., which was considered to be preserving to mitochondria function.
   As shown in FIGS. 34-38 and described below, mitochondria surprisingly and unexpectedly retained mitochondrial function after cold storage at −80° C., as determined by mitochondria size, complexity, mPTP opening, respiration, and gross morphology and the ability to reduce chemokine secretion in HPAEC.

Mitochondrial swelling was assessed using flow cytometry to measure FSC-A (size) and SSC-H (complexity) of mitochondria stored under non-preserving conditions (i.e., storage at 4° C.) or preserving conditions (i.e., storage at −80° C.). Mitochondria were determined to have a swelling phenotype if they had increased size and decreased complexity. While mitochondria stored at 4° C. almost immediately displayed a swelling phenotype (i.e., increased size, decreased complexity), mitochondria stored at −80° C. retained a normal phenotype comparable to freshly isolated mitochondria throughout the duration of storage (out to 7 months) (FIG. 34A).

Mitochondria mPTP opening was measured using flow cytometry. Mitochondria were stained with 4 μM calcein-AM. The stained mitochondria were collected up to 30,000 events, excited with a 488 nm laser, and assessed on FITC emission. Mitochondria were determined to have a regulated mPTP if they were able to retain fluorescent calcein, resulting in FITC+ staining. Mitochondria were determined to have dysregulated, continuous opening if they were unable to retain fluorescent calcein, resulting in reduced FITC staining. While mitochondria stored at 4° C. (non-preserving conditions) lost the ability to regulate their mPTP opening, mitochondria stored at −80° C. (preserving conditions) controlled mPTP opening comparable to freshly isolated mitochondria throughout the duration of storage (out to 7 months) (FIG. 34B).

To evaluate mitochondria respiration of mitochondria stored under non-preserving conditions or preserving conditions, RCRs were determined using the Seahorse instrument. RCRs were calculated from the OCR during ADP-stimulated RCR and uncoupled respiration (RCRmax). The OCR during each of these two states was divided by the basal OCR to obtain the OCR ratio. Maximal respiration was achieved by injecting the mitochondrial protonophore uncoupler BAM15. The ADP-stimulated respiration rates and uncoupled respiration rates of mitochondria stored at 4° C. declined over time, while mitochondria stored at −80° C. had ADP-stimulated respiration rates (FIG. 34C) and uncoupled respiration rates (FIG. 34D) comparable to freshly isolated mitochondria throughout the duration of storage (out to 6 weeks).

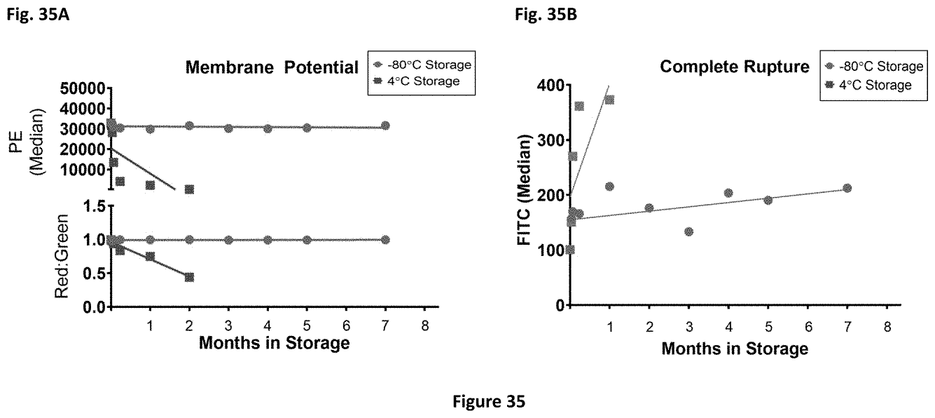

Changes in mitochondria membrane potential of mitochondria stored under non-preserving conditions (storage at 4° C.) or preserving conditions (storage at −80° C.) were assessed by flow cytometry using the JC-1 assay. Mitochondria were stained with 2 μM JC-1, 2 collected up to 30,000 events excited with a 488 nm laser, and assessed on FITC and PE emission. JC-1 dye exhibits potential-dependent accumulation in mitochondria, indicated by a fluorescent emission shift from green/FITC (˜529 nm) to red/PE (˜590 nm). The membrane potential-sensitive color shift is due to concentration-dependent formation of red fluorescent J-aggregates. Mitochondria depolarization is indicated by a decrease in the red:green fluorescence intensity ratio or by a decrease in the signal intensity in the PE (red) channel. While mitochondria stored at 4° C. showed a dramatic reduction in membrane potential, mitochondria stored at −80° C. retained membrane potential comparable to freshly isolated mitochondria throughout the duration of storage (out to 7 months) (FIG. 35A).

Permeability of mitochondria stored under non-preserving conditions or preserving conditions was measured by flow cytometry using a SYTOX green nucleic acid stain, which easily permeates mitochondria with comprised membranes. Damaged mitochondria stained with SYTOX green will have higher FITC signal intensity than non-damaged mitochondria stained with SYTOX green. While mitochondria stored at 4° C. had an immediate increase in FITC emission, mitochondria stored at −80° C. retained membrane potential comparable to freshly isolated mitochondria through the duration of storage (out to 7 months) (FIG. 35B).

To determine whether the changes in the characterization parameters translate to changes in functional capabilities, the ability of stored mitochondria to reduce chemokine secretion in HPAEC was assessed using a menadione-induced ROS overproduction model. HPAEC were cultured with 25 μM Menadione concurrently with or without mitochondria treatment at 50 particles/cell for 5 hours prior to assessment on all parameters. Mitochondria used in these experiments were stored under either non-preserving conditions (storage at 4° C.) or preserving conditions (storage at −80° C.) for 0 hours (fresh mitochondria), 24 hours, 48 hours, and 72 hours. Chemokines in the culture media of treated HPAEC were measured by flow cytometry using the LEGENDplex™ Human Proinflammatory Chemokine Panel (BioLegend®, San Diego, Calif.), which is a bead-based immunoassay. Beads were differentiated by size and internal fluorescent intensities. Each bead set was conjugated with a specific antibody on its surface and served as the capture beads for a specific analyte (chemokine). When a selected panel of capture beads was mixed and incubated with a sample containing target analytes specific to the captured antibodies, each analyte would bind to its specific capture beads. After washing, a biotinylated detection antibody cocktail was added, and each detection antibody in the cocktail would bind to its specific analyte bound on the capture beads, thus forming capture bead-analyte-detection antibody sandwiches. Streptavidin-phycoerythrin (SA-PE) was subsequently added, which would bind to the biotinylated detection antibodies, providing fluorescent signal intensities in proportion to the amount of bound analytes. Since the beads were differentiated by size and internal fluorescent intensity on a flow cytometer, analyte-specific populations could be segregated, and PE fluorescent signal could be quantified. The concentration of the analyte of interest was determined using a standard curve generated in the same assay.

The chemokines analyzed by the bead-based immunoassay include IL-8/CXCL8, MIG/CXCL9, MCP-1/CCL2, and GROα/CXCL1. IL-8 is a chemoattractant cytokine (i.e., chemokine) with distinct specificity for the neutrophil. IL-8 attracts neutrophils to sites of inflammation where it then helps to activate them. MIG is a chemokine that plays an important role in recruiting activated T cells to sites of inflammation. MIG participates in Th1/Th2 polarization (attracting Th1 cells and inhibiting Th2 migration). MIG is produced following an amplification of the IFN-γ signal and may serve as a useful readout for activation. MCP-1 is a chemokine that controls recruitment of monocytes and macrophages to sites of inflammation. GROα is a chemokine that controls recruitment of neutrophils in the early stages of inflammation.

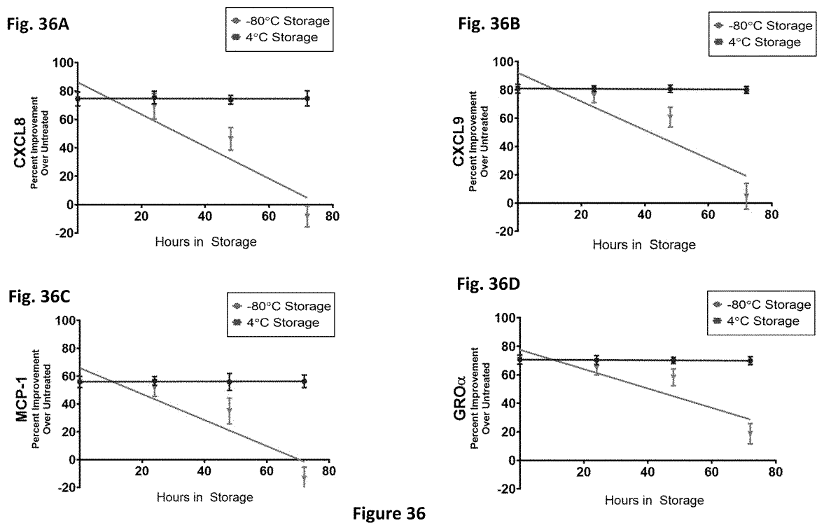

Results of the bead-based immunoassay are presented in FIG. 36 as the percent improvement over cells treated with 25 μM menadione+0 mitochondria/cell. Mitochondria stored at 4° C. rapidly lost their ability to modulate secretion of IL-8/CXCL8 (FIG. 36A), MIG/CXCL9 (FIG. 36B), MCP-1/CCL2 (FIG. 36C), and GROα/CXCL1 (FIG. 36D) compared to mitochondria stored at −80° C., which retained the ability to reduce chemokine secretion. These results show that isolated porcine mitochondria retain mitochondrial function after cold storage at −80° C.

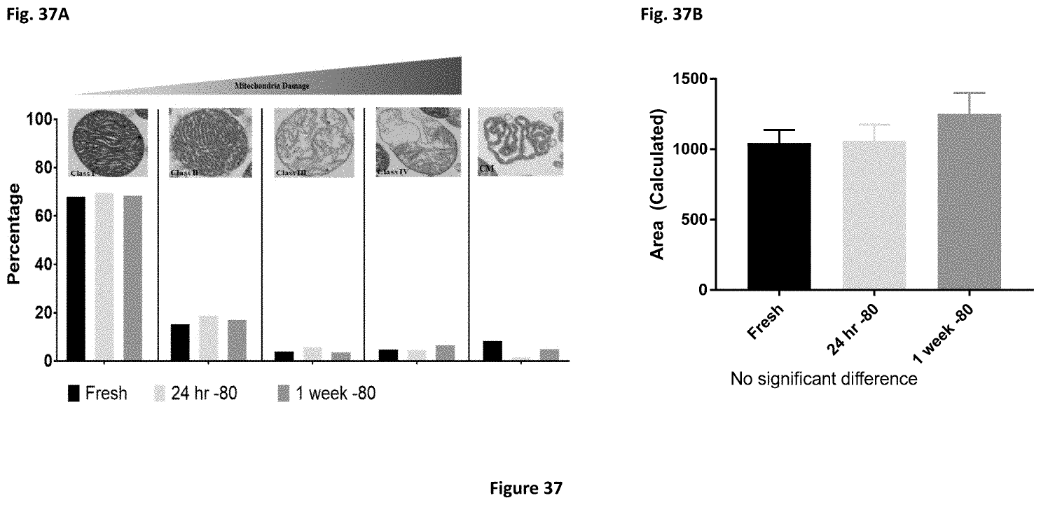

As shown in FIG. 37, mitochondria stored at −80° C. have the same gross morphology (FIG. 37A) and average size (FIG. 37B) as freshly isolated mitochondria. Mitochondria scored as class I had a condensed, normal state (i.e., non-damaged state) represented by numerous narrow pleomorphic cristae in a contiguous electron-dense matrix space. Mitochondria scored as class II were in a state of remodeling characterized by reorganized cristae and matrix spaces. The appearance of the remodeling state is temporally correlated with the redistribution and availability of cytochrome c from the intermembrane space. Mitochondria scored as class III were swollen and damaged. Class III mitochondria had intact membranes, but the cristae of these mitochondria have deteriorated and gathered close to the perimeter of the mitochondria. Mitochondria scored as class IV were terminally swollen or ruptured. Class IV mitochondria showed gross morphological derangement, including asymmetric blebbing of matrix. Mitochondria scored as “condensed matrix (CM)” had a condensed matrix with no limiting outer membrane.

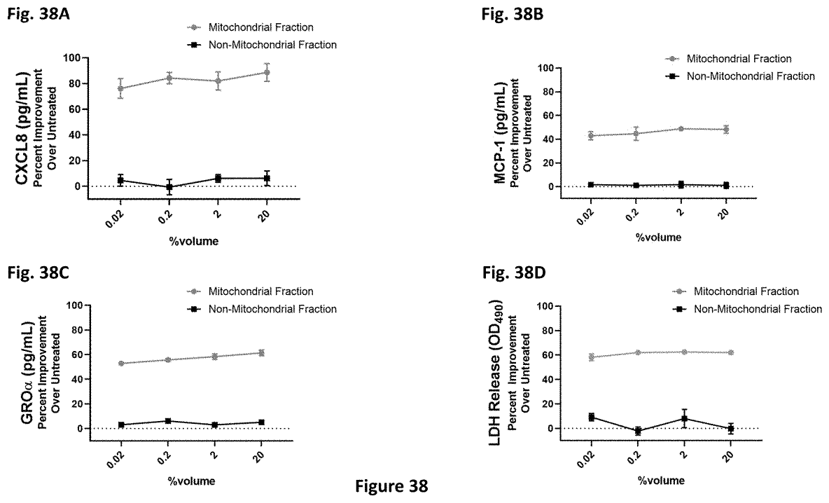

To assess whether intact mitochondria are the functional component in the mitochondria treatment, mitochondrial and non-mitochondrial fractions were obtained by centrifugation from mitochondria stored for two weeks at −80° C. HPAEC were cultured with 25 μM menadione and treated volumetrically with either the mitochondria fraction or the non-mitochondria fraction. The volumes of 0.02%, 0.2%, 2%, and 20% correspond to 1 mitochondria/cell, 10 mitochondria/cell, 100 mitochondria/cell, and 1,000 mitochondria/cell, respectively. Parameters analyzed included secretion of the inflammatory chemokines IL-8/CXCL8 (FIG. 38A), MCP-1/CCL-2 (FIG. 38B), and GROα/CXCL-1 (FIG. 38C), as well as lactate dehydrogenase (LDH) release (FIG. 38D), which is indicative of cell damage. All results are presented in FIG. 38 as the percent improvement over HPAEC treated with 25 μM menadione and 0 mitochondria/cell (0% volume). The mitochondrial fraction alone retained the ability to reduce chemokine secretion and LDH release. Therefore, mitochondria are the functional component in mitochondria treatment as opposed to a component released from the mitochondria after storage at −80° C. or carried over from the isolation process.

### Example 10

### Isolated Porcine Mitochondria Stored Long Term Under Preserving Conditions Improve Kidney Function and Recovery In Vivo

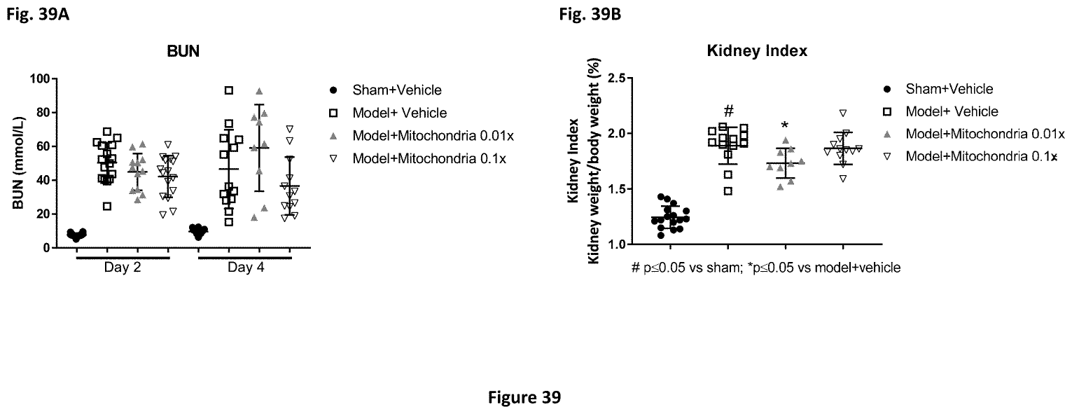

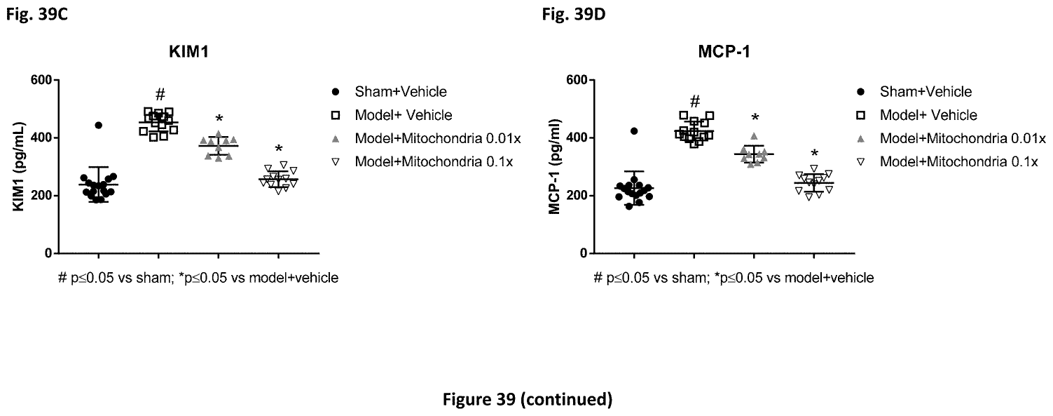

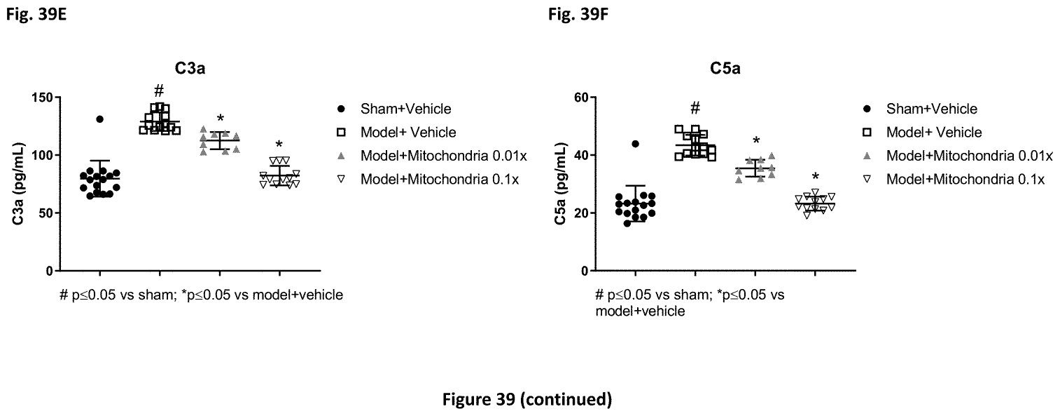

An ischemia/reperfusion (I/R) mouse model was used to assess the ability of isolated porcine mitochondria stored under long-term preserving conditions to improve kidney function and recovery in vivo. The mitochondria used in this study were stored for approximately one month at −80° C. (preserving conditions) prior to injection into mice. Acute I/R injury was achieved in adult mice by clamping the renal artery for 45 minutes followed by reperfusion. Mice were injected with mitochondria (0.01× or 0.1× dose) or the vehicle control upon reperfusion on day 1. As shown in FIG. 39A, blood urea nitrogen (BUN), which is an indicator of kidney function, was increased after I/R injury and trended to decrease at day 2 and on day 4 after mitochondria injection (0.1× dose). Kidney index, which is the percent mouse weight taken up by the kidney, was increased after I/R injury and was reduced after mitochondria injection (0.01× dose), as shown in FIG. 39B. Kidney injury molecule-1 (KIM1) is a marker of acute kidney injury. FIG. 39C shows that while I/R injury increased KIM1 serum levels, mitochondria treatment reduced these levels in a dose-responsive manner. Monocyte chemoattractant protein 1 (MCP1) is a proinflammatory cytokine associated with acute kidney injury. FIG. 39D shows that while FR injury increased MCP1 serum levels, mitochondria treatment reduced these levels in a dose-responsive manner. The C3a and C5a members of the compliment system induce inflammatory mediators from both renal tubular epithelial cells and macrophages after hypoxia/reoxygenation. While I/R injury increased serum levels of C3a (FIG. 39E) and C5a (FIG. 39F), mitochondria treatment reduced these levels in a dose-dependent manner (FIG. 39E-F).

Altogether, these data show that isolated porcine mitochondria stored long-term under preserving conditions can be administered to a subject to improve kidney function and recovery after injury.

### Example 11

### Treatment with Isolated Porcine Mitochondria Improves Lung Function after Injury

To prepare lungs for ex vivo lung perfusion (EVLP), porcine lungs were inflated, and the trachea clamped. Lungs were stored at 4 degrees Celsius for approximately 20 hours prior to the EVLP. The pulmonary artery (PA) and main bronchus (trachea) were cannulated on ice. The left atrium (LA) was kept open to allow for free efflux of perfusate from the lung. During the 5-hour EVLP, lungs were ventilated and perfused with 37° C. Steen solution, which was buffered with bicarbonate and deoxygenated with 5% CO₂, 95% N₂. Volume controlled ventilation was used with a pressure cap of 25 cmH₂O. PA perfusion was started at 100 ml/min and increased to 30% cardiac output over approximately 30-45 minutes. Perfusion was held constant during the EVLP. Before mitochondria injection, gas exchange was assessed by measuring PA and LA PO₂ at an FiO₂ of 100%. Physiological parameters, such as dynamic compliance, were also recorded at this time. Mitochondria or respiration buffer (control) were injected into the PA line immediately following baseline and after 3 hours of EVLP. EVLP assessments, including gas exchange, were made at 15 minutes, 1 hour, 2 hour, 3 hour, 4 hour and 5 hours post-baseline. Mitochondria used in these experiments were stored under preserving conditions (storage at −80° C.) prior to use (between 24 hours and 1 month in storage).

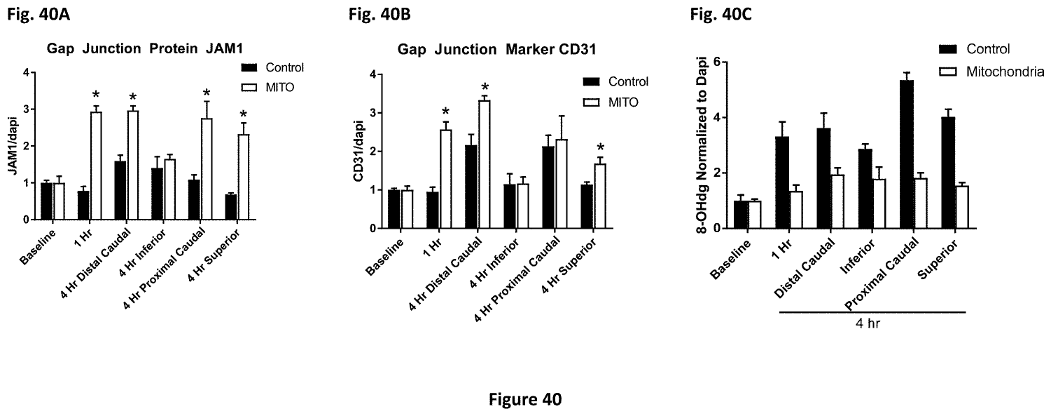

EVLP was run on isolated porcine cadaveric lungs after approximately 20 hours of cold ischemia time. As shown in FIG. 40, porcine mitochondria treatment improved the expression of gap junction markers and reduced DNA oxidation in an isolated porcine cadaveric lung placed on EVLP following cold ischemic injury. In particular, mitochondria treatment improved expression of the gap junction markers JAM1 (FIG. 40A) and CD31 (FIG. 40B) in EVLP after 1 hour in the superior lobe and after 4 hours when measured in the distal segment of the caudal lobe, the proximal segment of the caudal lobe, and the superior lobe. Mitochondria treatment also decreased expression of the ROS-induced DNA activation marker 8-OHdG in lung tissue during EVLP after 1 hour in the superior lobe and after 4 hours when measured in the distal segment of the caudal lobe, the proximal segment of the caudal lobe, the inferior lobe, and the superior lobe (FIG. 40C).

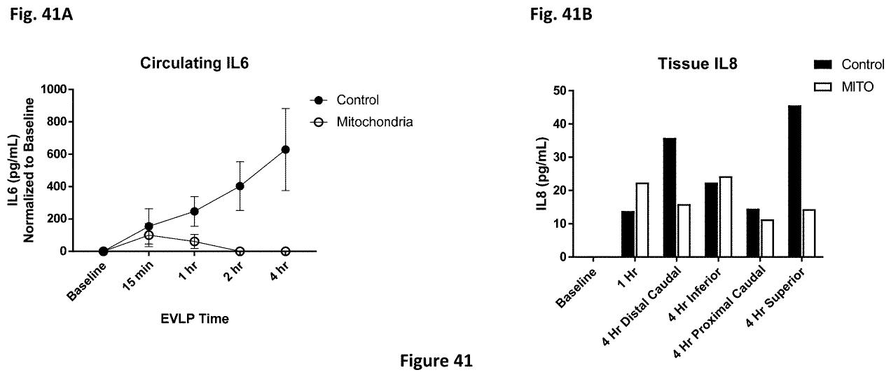

Lung tissue was stored overnight at 4° C., following which a lung tissue homogenate was made. Homogenate was treated with increasing doses of mitochondria and incubated at standard culture conditions overnight. As shown in FIG. 41A-B, porcine mitochondria treatment reduced inflammatory cytokine expression or secretion in isolated porcine cadaveric lungs following cold ischemic injury. In particular, mitochondria treatment decreased circulating IL-6 during EVLP (FIG. 41A) and decreased lung tissue lysate levels of IL-8 after 1 hour EVLP in the superior lobe and after 4 hours EVLP in the distal segment of the caudal lobe, the proximal segment of the caudal lobe, and the superior lobe (FIG. 41B).

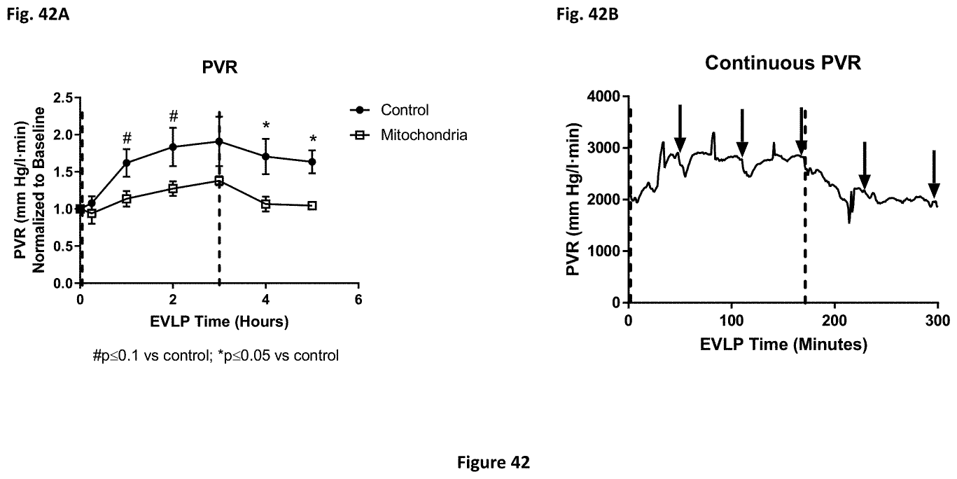

Pulmonary vascular resistance (PVR) of isolated porcine cadaveric lungs was measured during EVLP. Six lungs (“Control”) were treated with vehicle at the EVLP time of 3 hours, and five lungs were treated with mitochondria (“Mitochondria”) at the EVLP time of 3 hours were included in the analysis (FIG. 42A). A single mitochondria-treated lung is shown in FIG. 42B to demonstrate how mitochondria injection can be visually seen at the 3-hour injection. The dotted lines in FIG. 42A and FIG. 42B represent the time of mitochondrial injection. The arrows in FIG. 42B represent the times at which gas exchange was assessed. Between each assessment was a recruitment event. These results show that mitochondria injection during EVLP improved lung function by decreasing PVR.

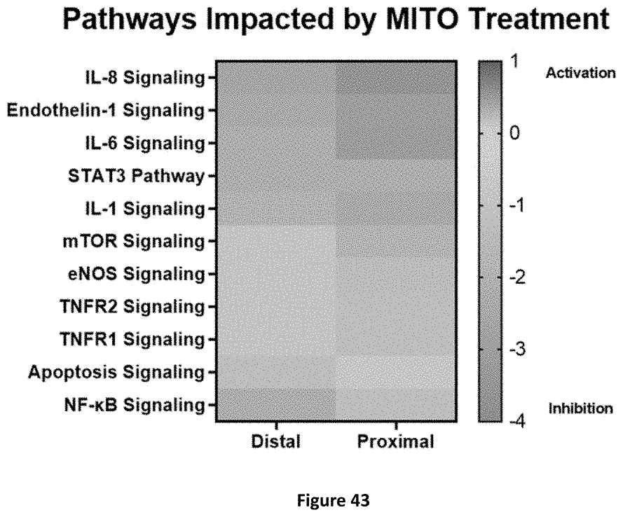

The impact of mitochondria treatment on signaling pathways was also evaluated. Isolated porcine cadaveric lungs were exposed to approximately 20 hours of cold ischemia time, after which EVLP was run on the lungs for 5 hours. Distal caudal and proximal caudal lung tissue was collected from control buffer injected or mitochondrial injected lungs and subjected to RNA sequencing. As shown in FIG. 43, mitochondria treatment decreased inflammatory and apoptotic signaling pathways in lungs placed on EVLP after cold ischemic injury.

Altogether, these results show that treatment of lungs with isolated porcine mitochondria improves lung function following injury by increasing expression of gap junction markers and by reducing DNA oxidation, inflammatory cytokine production, apoptosis, and PVR. As such, porcine mitochondria treatment can be implemented during EVLP to improve lung viability and function.

### Example 12

### Mitochondria Treatment Improves the Viability and Function of Cells or Organ Tissue Exposed to Damaging or Distressful Conditions

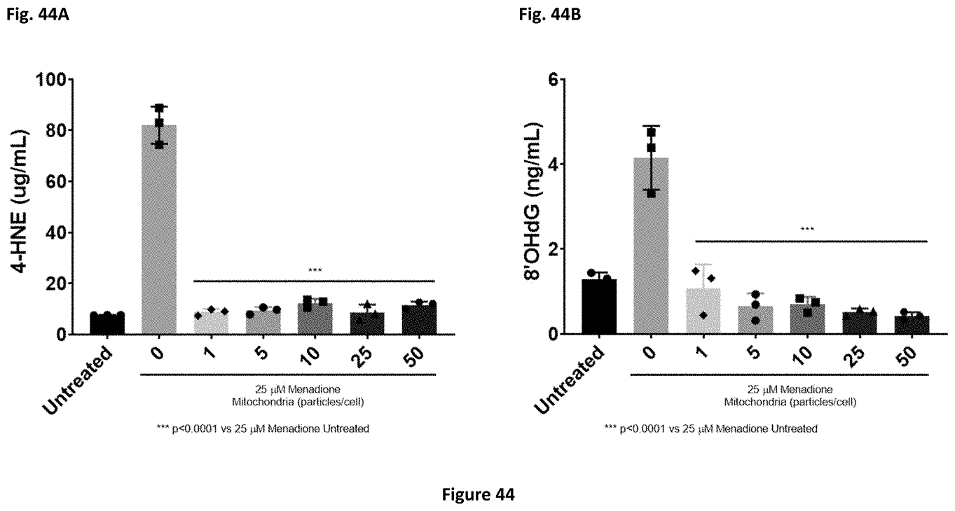

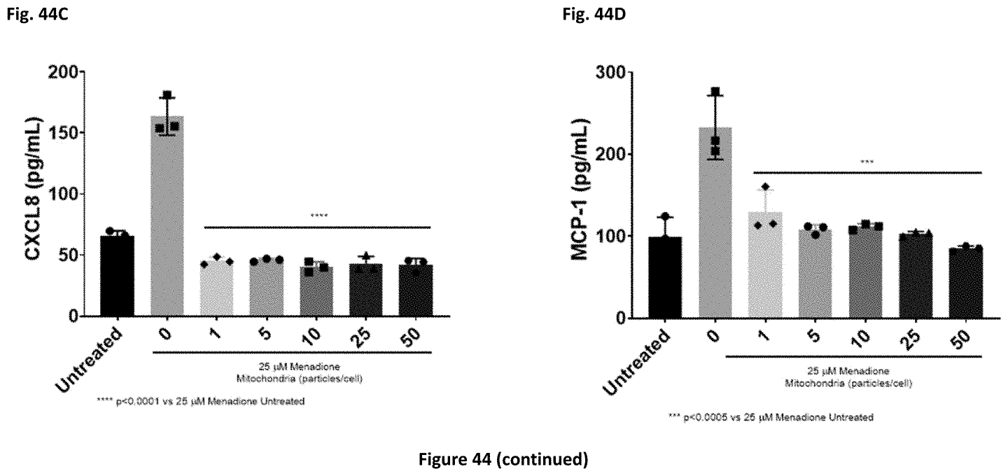

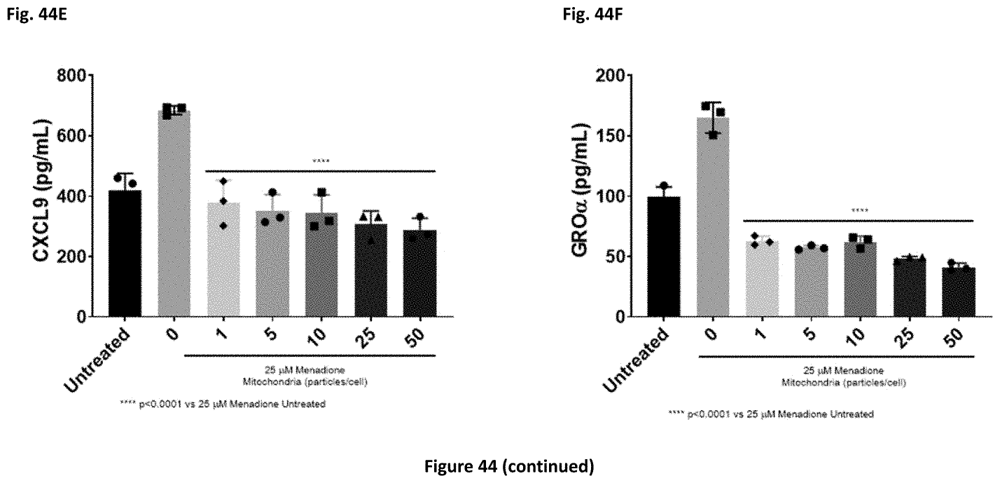

Healthy cells require tightly regulated amounts of ROS to function normally. When ROS generation is increased past a certain level, it becomes damaging to the cells and creates ROS-mediated damage to cellular components, including nucleic acids, lipids, and proteins. To evaluate the effects of mitochondria treatment on ROS generation, HPAEC were cultured with 25 μM of the ROS-inducer menadione with or without mitochondria treatment for 5 hours. The oxidative stress markers 4-HNE and 8-OHdG were measured in lysates of the treated cells by competitive ELISA. As shown in FIG. 44A-B, mitochondria treatment effectively reduced levels of 4-HNE adducts and 8-OHdG to normal (no menadione treatment) levels. Cell culture supernatants of the treated cells were analyzed for the presence of secreted chemokines by flow cytometry. As shown in FIG. 44C-E, mitochondria treatment effectively reduced secretion of IL-8/CXCL8, MCP1/CCL2, MIG/CXCL9, and GROα/CXCL1 to normal (no menadione treatment) levels. The mitochondria used for these experiments were stored at −80° C. for 1 week prior to use.

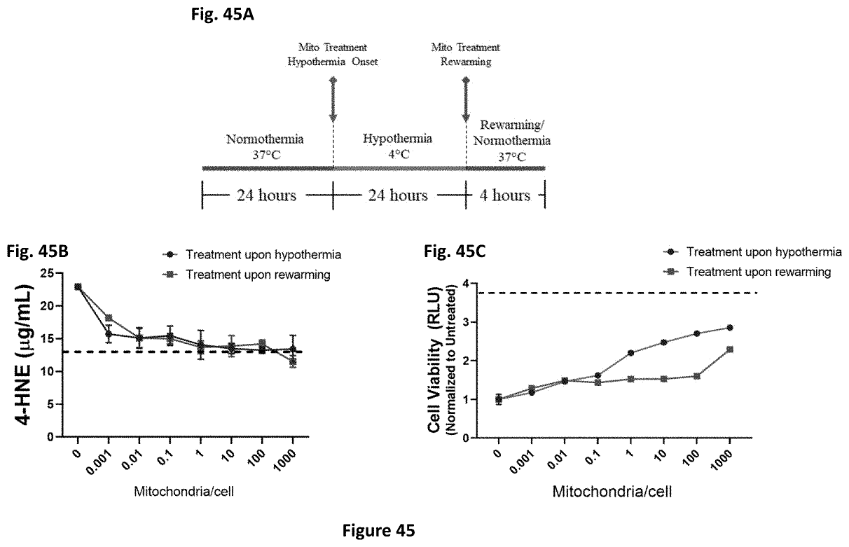

In addition to ROS-mediated injury, organs slated for transplantation often sustain cold/rewarming injury. At a cellular level, when the temperature decreases, the cells alter their metabolic functions and deplete their ATP stores. As the cell or organ is rewarmed, ATP demand and consumption increases. At the mitochondrial level, there is a prolonged opening of the mPTP, with a subsequent loss of membrane potential and an increase in oxidative stress. As the cell has depleted its stores of ATP, it is not prepared to jumpstart regular cellular metabolism, which results in cellular injury, initiation and activation of the necrosome, and eventual death/rupture and leakage of cellular contents resulting in a strong inflammatory response. To replicate this mode of injury in a two-dimensional (2D) culture model, HPAEC were cultured at 4° C. for 24 hours (hypothermic conditions) and rewarmed at 37° C. for 4 hours (normothermic conditions), as shown in FIG. 45A. The treatment groups included HPAEC treated with mitochondria at the onset of hypothermia and HPAEC treated with mitochondria at rewarming. After the 4-hour rewarming period, ROS-mediated damage was measured using a 4-HNE adduct competitive ELISA for quantitation of 4-HNE protein adducts in HPAEC lysates. 4-HNE adduct formation was very sensitive to mitochondria treatment as very low doses of mitochondria were able to have an impact (FIG. 45B). Cellular viability was also measured after the 4-hour rewarming period. Results are shown in FIG. 45C as relative light units (RLU) normalized to baseline (i.e., HPAEC exposed to cold/rewarming with no mitochondria treatment). Mitochondria treatment produced a 2-3 fold increase in cellular viability compared to untreated HPAEC (FIG. 45C).

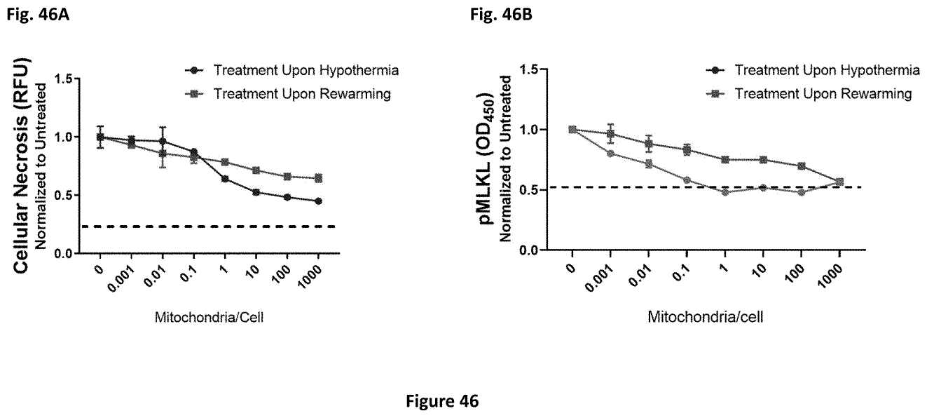

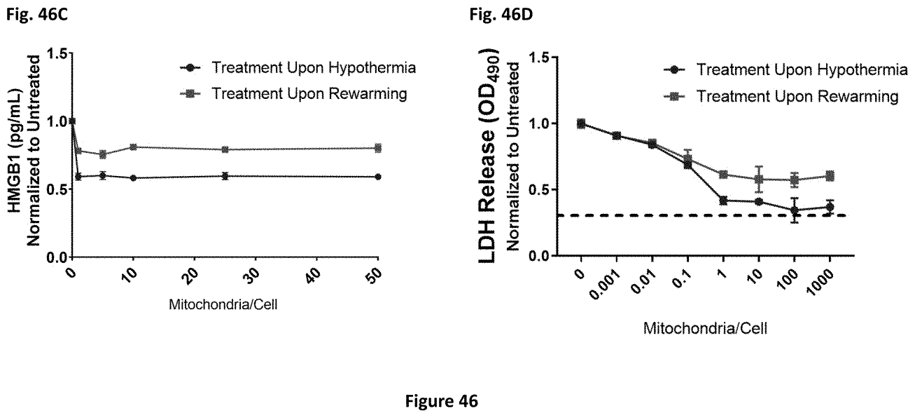

The effect of mitochondria treatment on necrosis of HPAEC subjected to cold/rewarming injury was also assessed using the 2D culture model shown in FIG. 45A. The treatment groups included HPAEC treated with mitochondria at the onset of hypothermia and HPAEC treated with mitochondria at rewarming. After the 4-hour rewarming period, necrotic cell death was measured using a cell-impermeant, profluorescent DNA dye. Live cells will exclude this dye, but necrotic cells which have compromised membrane integrity will allow entry of the dye. Results are shown in FIG. 46A as relative light units (RLU) normalized to baseline (i.e., HPAEC exposed to cold/rewarming with no mitochondria treatment). Normal, unstressed HPAEC controls are represented in FIG. 46A by a dashed line. HPAEC treated with mitochondria showed a dose-dependent decrease in necrosis (FIG. 46A).

A hallmark of necrotic cell death is the phosphorylation of MLKL. HPAEC lysates collected after the 4-hour warming period in the 2D culture model shown in FIG. 45A were analyzed using a sandwich ELISA to measure phospho-MLKL (pMLKL) and total MLKL. An anti-pan MLKL antibody was coated onto a 96-well plate. In select wells, rabbit anti-phospho-MLKL (Ser358/345) antibody was added to detect phosphorylated MLKL. In the remaining wells, rabbit anti-pan MLKL antibody was used to detect pan MLKL. Results are shown in FIG. 46B as optical density measured at a wavelength of 450 nm (OD₄₅₀) normalized to baseline (i.e., HPAEC exposed to cold/rewarming with no mitochondria treatment). Normal, unstressed HPAEC controls are represented in FIG. 46B by a dashed line. HPAEC treated with mitochondria showed a dose-dependent decrease in pMLKL levels (FIG. 46B). Total MLKL levels were unchanged (data not shown).

High Mobility Group Box 1 (HMGB-1) is a ubiquitous nuclear protein passively released by cells undergoing necrosis. Released HMGB-1 in HPAEC culture supernatants in the 2D culture model shown in FIG. 45A was measured by sandwich ELISA. The results shown in FIG. 46C were normalized to baseline (i.e., HPAEC exposed to cold/rewarming with no mitochondria treatment). Mitochondria treatment reduced HMGB-1 release compared to untreated cells (FIG. 46C). Lactate dehydrogenase (LDH) is a stable cytosolic enzyme that is released upon cell lysis. Released LDH in HPAEC culture supernatants was measured with a 30-minute coupled enzymatic assay, which results in conversion of a tetrazolium salt (INT) into a red formazan product. Results are shown in FIG. 46D as optical density measured at a wavelength of 490 nm (OD₄₉₀) normalized to baseline (i.e., HPAEC exposed to cold/rewarming with no mitochondria treatment). Normal, unstressed HPAEC controls are represented in FIG. 46C by a dashed line. Mitochondria treatment reduced LDH release compared to untreated cells (FIG. 46D).

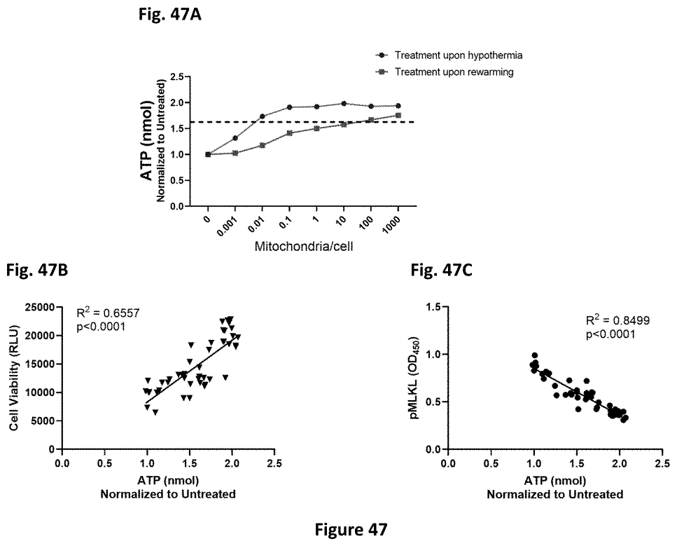

The effect of mitochondria treatment on total cellular levels of ATP in HPAEC subjected to cold/rewarming injury was also assessed by luminescent ATP detection assay using cell lysates obtained from the 2D culture model shown in FIG. 45A. The luminescent ATP detection assay allows the detection of total levels of cellular ATP and is based on the production of light caused by the reaction of ATP with added firefly luciferase and luciferin. The emitted light is proportional to the ATP concentration inside the cells. ATP degrading enzymes (i.e., ATPases) were irreversibly inactivated during the cell lysis step of this assay to ensure that the luminescent signal obtained truly corresponds to the endogenous levels of ATP. The treatment groups included HPAEC treated with mitochondria at the onset of hypothermia and HPAEC treated with mitochondria at rewarming, and total levels of cellular ATP were measured after the 4-hour rewarming period. The results shown in FIG. 47A were normalized to baseline (i.e., HPAEC exposed to cold/rewarming with no mitochondria treatment). Mitochondria treated HPAEC had increased ATP concentrations compared to untreated cells (FIG. 47A). There was a positive correlation between increased ATP concentration and cell viability (FIG. 47B) and a negative correlation between increased ATP concentration and necrosis (FIG. 47C). These results show that mitochondria treatment increases total levels of cellular ATP in cells subjected to cold/rewarming injury, which correlates with improved cell viability

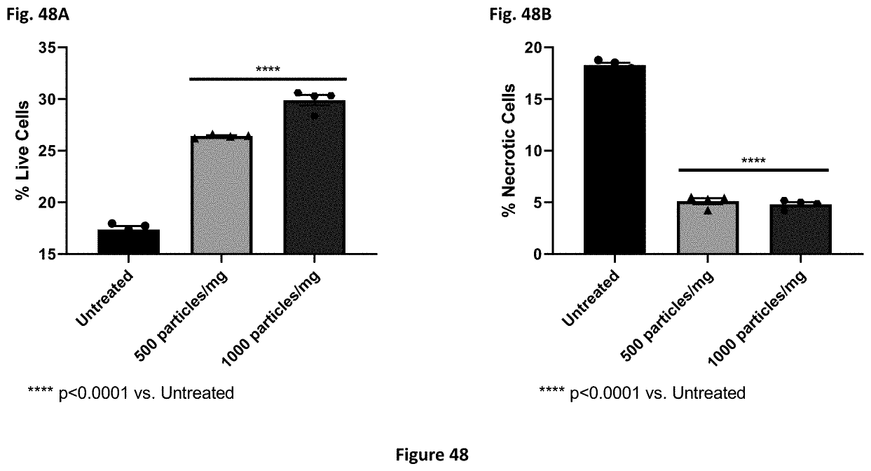

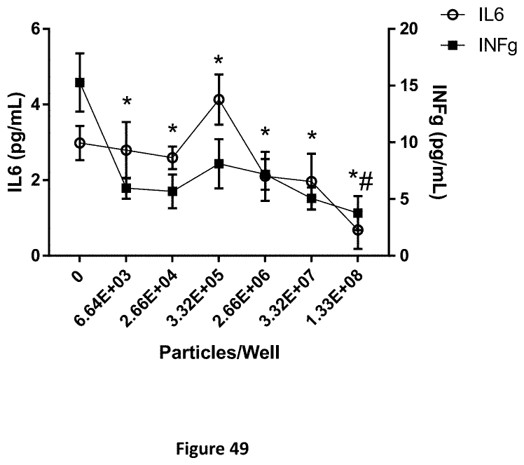

The effects of mitochondria treatment on cell viability, necrosis, and cytokine secretion were also evaluated in lung homogenates. To evaluate cell viability and necrosis, distal pieces of lungs were collected after 24 hours in cold storage, enzymatically digested in a 0.1 mg/mL DNaseI/0.01% collagenase solution, treated with mitochondria, and placed under normothermic (rewarming) cell culture conditions for 4 hours prior to assessment. Mitochondria treatments (500-particles/mg or 100-particles/mg) were based on wet tissue weight and were similar to the dosing used in the EVLP experiments described herein. Compared to untreated lung homogenates, mitochondria treatment significantly improved cell viability (FIG. 48A) and reduced necrosis (FIG. 48B). To evaluate cytokine secretion, lung tissue was stored overnight at 4° C., following which a lung tissue homogenate was made. Homogenate was treated with increasing doses of mitochondria and incubated at standard culture conditions (37° C.) overnight. As shown in FIG. 49, mitochondria treatment decreased secretion of IL-6 and IFN-γ after cold exposure and homogenization.

Altogether, the results show that mitochondria treatment reduces ROS-mediated oxidative byproduct production, ROS-mediated chemokine secretion, and cold/rewarming injury of cells and organ tissue. As such, there is an advantage to treating cells or organ tissue with mitochondria prior to or after exposure of the cells or organ tissue to damaging or distressful conditions, such as hypothermia.

1. A method of organ transplantation, the method comprising delivering isolated mitochondria to an organ intended for transplantation.

2. (canceled)

3. The method of claim 2, wherein the isolated mitochondria are delivered to the organ prior to harvesting the organ from the donor.

4. (canceled)

5. (canceled)

6. The method of claim 1, wherein the isolated mitochondria are isolated human mitochondria allogeneic to the recipient.

7. (canceled)

8. The method of any one of claim 1, wherein the organ intended for transplantation is harvested from a human donor.

9. (canceled)

10. The method of claim 8, wherein the isolated mitochondria are isolated human mitochondria autologous to the human donor.

11. The method of claim 1, wherein the organ intended for transplantation is engineered from a porcine organ scaffold.

12. The method of claim 1, wherein the isolated mitochondria are isolated porcine mitochondria.

13. The method of claim 1, wherein cells of the organ treated with the isolated mitochondria have at least 5% improvement in mitochondrial function in comparison to cells of a corresponding organ not treated with the isolated mitochondria.

14. The method of claim 13, wherein the improved mitochondrial function is increased oxygen consumption and/or increased adenosine triphosphate (ATP) synthesis.

15. (canceled)

16. The method of claim 1, wherein the organ is a lung.

17. The method of claim 16, wherein the lung treated with the isolated mitochondria is transplanted into a human recipient suffering from a lung disease or disorder.

18. The method of claim 17, wherein the lung disease or disorder is pulmonary hypertension, bronchopulmonary dysplasia (BPD), lung fibrosis, asthma, sleep-disordered breathing, or chronic obstructive pulmonary disease (COPD).

19-23. (canceled)

24. A method of improving the performance of an implanted tissue or transplanted organ in a subject, the method comprising delivering isolated mitochondria to a tissue or organ before, during, or after implantation or transplantation of the tissue or organ, wherein the tissue or organ is a donor tissue, donor organ, engineered tissue, or engineered organ.

25-36. (canceled)

37. A method of improving the function of a lung subjected to ex vivo lung perfusion (EVLP), the method comprising:
(i) delivering isolated mitochondria to a lung, and
(ii) performing EVLP on the lung in a chamber or vessel by perfusing the lung with a perfusate solution from a reservoir.

38-68. (canceled)

69. A method for minimizing damage to an organ ex vivo due to cold ischemia during transportation, shipment, or storage, the method comprising delivering isolated mitochondria to the organ 0-24 hours before cold ischemia, during cold ischemia, or 0-24 hours after cold ischemia,
wherein cells of the organ treated with the isolated mitochondria have at least 5% improvement in mitochondrial function in comparison to cells of a corresponding organ not treated with the isolated mitochondria, and
wherein the improved mitochondrial function is increased oxygen consumption and/or increased ATP synthesis.

70-109. (canceled)

110. A method for improving the function of an engineered organ or tissue, the method comprising:
(i) preparing an organ or tissue scaffold comprising one or more extracellular matrix components,
(ii) populating the organ or tissue scaffold in a bioreactor, chamber or vessel with populating cells to produce an engineered organ or tissue, and
(iii) delivering isolated mitochondria to the engineered organ or tissue.

111-115. (canceled)

116. The method of claim 110, wherein the engineered organ or tissue treated with the isolated mitochondria is an engineered human kidney.

117-131. (canceled)

132. The method of claim 110, wherein the isolated mitochondria are delivered to the engineered organ or tissue after the step of populating the organ or tissue scaffold.

133-277. (canceled)

278. A method for treating a lung disease or disorder in a subject in need thereof, the method comprising:
(i) administering a therapeutically effective amount of a composition comprising isolated mitochondria to the subject, and
(ii) administering a therapeutically effective amount of a medication for treating the lung disease or disorder,
wherein the composition is administered to the subject before, concurrently with, or after the administration of the medication for treating the lung disease or disorder.

279. The method of claim 278, wherein the isolated mitochondria are isolated porcine mitochondria.

280. (canceled)

281. The method of claim 278, wherein the isolated mitochondria are isolated human mitochondria autologous to the subject.

282. The method of claim 278, wherein the lung disease or disorder is pulmonary hypertension, asthma, sleep-disordered breathing, BPD, COPD, or lung fibrosis.

283. (canceled)

284. The method of claim 278, wherein the medication for treating the lung disease or disorder is selected from the group consisting of: treprostinil, epoprostenol, iloprost, bosentan, ambrisentan, macitentan, and sildenafil.

285-441. (canceled)

442. A method for improving the cold transportation, cold shipment, or cold storage of isolated cells, the method comprising delivering isolated mitochondria to the isolated cells before, during, or after cold transportation, cold shipment, or cold storage,
wherein the cells treated with the isolated mitochondria have at least 5% improvement in viability in comparison to cells of corresponding cells not treated with the isolated mitochondria.

443-525. (canceled)


SEQUENCE LISTING (4 sequences)

SEQ ID NO: 1 - protein, 6 aa, Artificial Sequence
  Description of Artificial Sequence Synthetic 6xHis tag
HHHHHH

SEQ ID NO: 2 - DNA, 25 bp, Artificial Sequence
  Description of Artificial Sequence Synthetic oligonucleotide
cagcactatgtgcaatcacacaaaa

SEQ ID NO: 3 - DNA, 24 bp, Artificial Sequence
  Description of Artificial Sequence Synthetic oligonucleotide
tggttgatgccgattgtcactatt

SEQ ID NO: 4 - DNA, 19 bp, Artificial Sequence
  Description of Artificial Sequence Synthetic oligonucleotide
tcgtagccttctcaacttc